(19) United States
(12) Patent Application Publication
(10) Pub. No.: US 2022/0193262 A1
(43) Pub. Date: Jun. 23, 2022

(54) HIGH EFFICIENCY GENE DELIVERY SYSTEM

(71) Applicants: President and Fellows of Harvard College, Cambridge, MA (US); Massachusetts Eye and Ear Infirmary, Boston, MA (US); The Schepens Eye Research Institute, Inc., Boston, MA (US)

(72) Inventors: Michael Florea, Cambridge, MA (US); Amy J. Wagers, Cambridge, MA (US); Luk Vandenberghe, Weston, MA (US)

(21) Appl. No.: 17/666,543

(22) Filed: Feb. 7, 2022

Related U.S. Application Data
(63) Continuation of application No. PCT/US2021/029757, filed on Apr. 28, 2021.
(60) Provisional application No. 63/016,968, filed on Apr. 28, 2020.

(51) Int. Cl. A61K 48/00; C12N 15/86

(52) U.S. Cl. CPC A61K 48/005; C12N 15/86; C12N 2750/14143; A01K 2227/105; A01K 2267/035; A01K 2217/075

(57) ABSTRACT

The disclosure provides viral vector delivery systems for use in treating diseases or disorders in a subject to whom the viral vector delivery systems are administered, as well as to methods of making and using the viral vector delivery systems.

### RELATED APPLICATIONS

This application is a continuation of International Application No. PCT/US2021/029757, filed Apr. 28, 2021, which claims the benefit of U.S. Provisional Application No. 63/016,968, filed on Apr. 28, 2020. The entire teachings of the above applications are incorporated herein by reference. International Application No. PCT/US2021/029757 was published under Article 21(2) in English.

### STATEMENT REGARDING SEQUENCE LISTING

Sequence Listing associated with this application is provided in text format in lieu of a paper copy and is hereby incorporated by reference into the specification. The name of the text file containing the Sequence Listing is HRVY_174_101X.txt. The text file is 45 KB, was created on Feb. 4, 2022, and is being submitted electronically via EFS-Web, concurrent with the filing of the specification.

### BACKGROUND OF THE INVENTION

The field of aging has made great advances in the past few decades. Many pathways and genes whose modulation increases healthspan and longevity, and the first therapeutics targeting aging, are starting to emerge. However, most discoveries from aging studies cannot be translated to the clinic due to lack of appropriate small-molecule drugs, even for severe early-aging diseases. Furthermore, research into the genetics of aging using mice and other mammals has remained slow and expensive because it requires generation, breeding and aging of large cohorts of transgenic animals.

The lack of translatability and the time and cost of research are the main problems that are seen in the field of aging today. These problems cannot be solved using conventional methods. However, they could potentially be solved through the use of advanced gene therapy to directly perturb genes in aged animals and to deliver geroprotective genes into patients. However, this has not been achieved due to current limitations in gene delivery technologies.

### SUMMARY OF THE INVENTION

Described herein is a high-efficiency adeno-associated virus (AAV)-based body-wide gene therapy method to express or overexpress genes (e.g., geroprotective genes). The system is an AAV expression system for systemic expression (e.g., uniform systemic expression), e.g., a single or multi AAV expression system for uniform, systemic expression (DAEUS). It is shown herein that DAEUS can achieve overexpression of several geroprotective genes in aged wild-type mice. It is further shown herein that DAEUS can fully rescue Cisd2 expression in Wolfram Syndrome II mice, as well as retard and reverse major progeroid morbidities in these mice. DAEUS is a gene therapy platform that, among other uses, enables acceleration of studies into the basic biology of aging, the treatment of progerias, and the overexpression of geroprotective genes to extend healthspan and/or lifespan. Disclosed herein is a viral vector delivery system. The viral vector delivery system comprises two or more viral serotypes engineered for delivery of a single gene (i.e., the same gene is delivered by each of the two or more viral serotypes). In some embodiments, the viral vector delivery system comprises an unlimited number of viral serotypes for delivery of the single gene. For example, the viral vector delivery system may comprise at least 5, 10, 25, 50, 75, or 100 viral serotypes, or may comprise 2 to 20 or 5 to 10 viral serotypes.

In some embodiments, the viral serotypes are adeno-associated viral serotypes (e.g., AAV1, AAV2, AAV3, AAV3B, AAV4, AAV5, AAV6, AAV7, AAV8, AAV9, AAV10, AAV11, Anc80, AAVrh10, AAV-DJ, AAV-DJ/8, AAV-PHP.B, AAV-PHP.S, AAV-PHP.eB, AAV.CAP-B10, AAV.CAP-B22, and AAVMYO, etc.). In some embodiments each of the two or more viral serotypes is trophic for a different cell or tissue type (i.e., a first viral serotype is trophic for a first cell or tissue type, and a second viral serotype is trophic for a second cell or tissue type). In some embodiments, at least one viral serotype is AAV9. In some embodiments, at least one viral serotype is PHP.eB. In certain embodiments, a first viral serotype is AAV9 and a second viral serotype is PHP.eB. In some embodiments, a viral serotype is selected from Table 1.

The viral vector delivery system may further comprise a miRNA target site. In some embodiments, the miRNA target site is selected based on a tissue target, e.g., aorta, endothelium, cardiac muscle skeletal muscle, tongue, esophagus, stomach, small intestine, large intestine, diaphragm, eye, optic nerve, inner ear, auditory nerve, brown fat, white fat, central nervous system, peripheral nervous system, kidney, spleen, liver, lung, heart, brain, thymus, ovaries, testes, skin, pancreas, bone marrow cells, osteoblasts and osteoclasts, blood cells, hematopoietic stem cells, or muscle satellite cells, or more specifically, cardiac, liver, muscle, or brain tissue. In some embodiments, miRNA target site is selected from the group consisting of miRNA-1, miRNA-24, miRNA-29, miRNA-30c, miRNA-33, miRNA-122, miRNA-124, miRNA-128, miRNA-133, miRNA-144, miRNA-148a, miRNA-208a, miRNA-208b, miRNA-223, and miRNA-499. For example, a target tissue may be cardiac tissue and the miRNA target site may be miRNA-1, miRNA-133, miRNA-208a, miRNA-208b, or miRNA-499. In some embodiments, a target tissue is liver tissue and the miRNA target site is selected from the group consisting of miRNA-24, miRNA-29, miRNA-30c, miRNA-33, miRNA-122, miRNA-144, miRNA-148a, and miRNA-223. In some embodiments, a target tissue is muscle tissue and the miRNA target site is miRNA-1 or miRNA-133. In some embodiments, a target tissue is brain tissue and the miRNA target site is miRNA-124 or miRNA-128.

The viral vector delivery system may further comprise a non-silencing promoter. In some embodiments, the non-silencing promoter leads to RNA expression of at least 30%, or optionally at least 50%, of CMV promoter expression. In some embodiments, the promoter is selected from the group consisting of Cbh, CAG, CB7, and CBA. In certain embodiments, the promoter is Cbh.

In some embodiments, the viral vector delivery system optionally further comprises a self-complementary vector backbone.

In some embodiments, the gene to be delivered is selected from Table 2. In certain embodiments, the gene is selected from the group consisting of Cisd2, Atg5, and PTEN. In some embodiments, the gene is a geroprotective gene. In some embodiments, the gene is a gene associated with a disease or disorder in need of treatment in a subject, e.g., a gene whose expression is absent or reduced in a disease or disorder to be treated.

Also disclosed herein are pharmaceutical compositions comprising the viral vector delivery systems disclosed herein. Also disclosed herein are methods of treating or preventing a disease or disorder in a subject comprising administering the pharmaceutical compositions or viral vector delivery systems disclosed herein.

Disclosed herein are methods of delivering to and expressing in multiple (two or more) cell or tissue types of a subject the same gene relatively simultaneously, as well as methods of treating or preventing a disease or disorder. The methods comprise administering to a subject a viral vector delivery system comprising at least one viral serotype, at least two viral serotypes, at least three viral serotypes, at least four viral serotypes, or at least five viral serotypes engineered for delivery of a single gene. In some embodiments, the viral vector delivery system comprises an unlimited number of viral serotypes for delivery of the single gene.

In some embodiments, the disease or disorder is an aging related disease or disorder, e.g., progeria syndrome, Wolfram Syndrome, neurodegenerative disorder, neurovascular disorder, skeletal muscle conditions, Cowden syndrome, Bannayan-Riley-Ruvalcaba syndrome, Proteus syndrome, Proteus-like syndrome and other PTEN-opathies. Werner syndrome, Bloom syndrome, Rothmund-Thomson syndrome, Cockayne syndrome, xeroderma pigmentosum, trichothiodystrophy, combined xeroderma pigmentosum-Cockayne syndrome, restrictive dermopathy, diabetes, obesity, cardiovascular disease, cancer, ocular degeneration, liver failure, and age-related macular degeneration. In some embodiments, the disease or disorder would benefit from administration of the gene to two or more tissue targets. In certain embodiments, the disease or disorder is Wolfram Syndrome II.

In some embodiments, the gene is expressed in two or more tissues in the subject. The gene may be uniformly expressed or overexpressed across two or more tissues in the subject. In some embodiments, the gene is delivered to at least 50% of tissues in the subject, and in some embodiments, is expressed for at least 4 months in the subject.

Also disclosed herein is a viral vector delivery system comprising two or more AAV serotypes engineered for delivery of a single gene, a non-silencing promoter, at least one miRNA target site, the gene, and optionally a self-complementary backbone.

In some embodiments, the AAV serotypes are AAV9 and PHP.eB. In some embodiments, the gene is selected from the group consisting of Cisd2, Atg5, and PTEN, and preferably is Cisd2.

Methods of treating a disease or disorder, e.g., Wolfram Syndrome II, are also disclosed herein, comprising administering to a subject the viral vector delivery system disclosed herein.

Also disclosed herein are methods of extending the lifespan of a subject. For example, lifespan may be extended by administering the viral vector delivery system described herein or a pharmaceutical composition comprising the viral vector delivery system described herein (e.g., a viral vector delivery system comprising at least one, at least two, at least three, at least four, or more viral serotypes engineered for delivery of a single gene).

Further described herein are methods of treating Wolfram Syndrome II comprising administering an effective amount of Cisd2 to a subject suffering from Wolfram Syndrome II.

In some embodiments, Cisd2 is administered to the subject via gene therapy, e.g., via a viral vector delivery system or any other gene therapy known to those of skill in the art. In some embodiments, the viral vector delivery system comprises at least one viral serotype, at least two viral serotypes, at least three viral serotypes, at least four viral serotypes, at least five viral serotypes.

Also described herein are methods of identifying a pre-determined level of gene transfer in one or more target tissues of a subject comprising: obtaining a dose-response curve characterizing the relationship between an amount of a vector administered to the subject and a resulting gene transfer level in the one or more target tissues; obtaining a linear or non-linear equation charactering the relationship between the amount of vector administered to the subject and the resulting gene transfer level in the one or more target tissues; and interpolating or extrapolating a required dose of a gene delivery system to achieve a defined level of gene transfer in the one or more target tissues.

Further described herein are methods of identifying a pre-determined level of transgene expression in one or more target tissues of a subject comprising: obtaining a dose-response curve characterizing the relationship between an amount of a vector administered to the subject and a resulting transgene expression level in the one or more target tissues; obtaining a linear or non-linear equation charactering the relationship between the amount of vector administered to the subject and the resulting transgene expression level in the one or more target tissues; and interpolating or extrapolating a required dose of a gene delivery system to achieve a defined level of transgene expression in the one or more target tissues.

In some embodiments, the gene delivery system comprises at least one viral serotype, at least two viral serotypes, at least three viral serotypes, at least four viral serotypes, at least five viral serotypes. In some embodiments, the viral serotype is an adeno-associated viral serotype (e.g., AAV1, AAV2, AAV3, AAV3B, AAV4, AAV5, AAV6, AAV7, AAV8, AAV9, AAV10, AAV11, Anc80, AAVrh10, AAV-DJ, AAV-DJ/8, AAV-PHP.B, AAV-PHP.S, AAV-PHP.eB, AAV.CAP-B10, AAV.CAP-B22, AAVMYO, etc.). In some embodiments, the viral serotype is selected from Table 1. In some embodiments, the one or more target tissues comprise a single tissue or two or more tissues. In some embodiments, the one or more target tissues are selected from the group consisting of aorta, endothelium, cardiac muscle skeletal muscle, tongue, esophagus, stomach, small intestine, large intestine, diaphragm, eye, optic nerve, inner ear, auditory nerve, brown fat, white fat, central nervous system, peripheral nervous system, kidney, spleen, liver, lung, heart, brain, thymus, ovaries, testes, skin, pancreas, bone marrow cells, osteoblasts and osteoclasts, blood cells, hematopoietic stem cells, and muscle satellite cells.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1B demonstrates the results of Cisd2 deficiency in mice. FIG. 1A shows dose-dependent modulation of lifespan by Cisd2 in male mice. Cisd2 deficiency shortens the lifespan and causes premature aging in Cisd2 KO mice. In contrast, a persistent level of Cisd2 expression prolongs lifespan and increases the survival rate of Cisd2 TG mice. See Wu, et al. Hum. Mol. Genet. 21, 3956-3968 (2012). FIG. 1B provides images showing the decreased body weight, shortened life span, and the ocular and cutaneous symptoms of aging in Cisd2^{-/-} mice. Early depigmentation and gray hair are seen on the top of the head and on the shoulders, and the prominent eyes and protruding ears of the Cisd2^{-/-} mice are also shown. See Chen, et al. Genes Dev. 23, 1183-1194 (2009).

FIGS. 2A-2D provide an overview of ssAAV9. FIG. 2A provides an ssAAV9 vector overview. FIG. 2B shows ssAAV9 DNA biodistribution at a dose of ˜1e12 vg/mouse (ssAAV9-Atg5 and ssAAV9-Cisd2 denoted as ssAAV9). FIGS. 2C-2D show lack of global overexpression on the protein level for Atg5 (FIG. 2C) or Cisd2 (FIG. 2D). 8 week old wild-type C57BL6/J mice were injected and euthanized 28 days post-injection. Cisd2 and Atg5 levels were determined via Simple Wes.

FIGS. 3A-3E demonstrate poor systemic overexpression of rejuvenation genes Oct4-Sox2-K1f4 using conventional ssAAV9 vectors. (See Lu et al 2019). FIG. 3A shows Sox2 expression in the liver of WT mice post-intravenous delivery of OSK-AAV9 and OSK transgenic (TG) mice. FIG. 3B shows body weight of WT mice and AAV-mediated OSK-expressing mice (1.0×10\hat{ }12 gene copies total) with or without doxycycline in the following 9 months after 4 weeks of monitoring (n=5,3,6,4 respectively). FIG. 3C shows AAV-UBC-rtTA and AAV-TRE-Luc vectors used for measuring tissue distribution. FIG. 3D shows Luciferase imaging of WT mice at 2 months after retroorbital injections of AAV9-UBC-rtTA and AAV9-TRE-Luc (1.0×10\hat{ }12 gene copies total). Doxycycline was delivered in drinking water (1 mg/mL) for 7 days to the mouse shown on the right. FIG. 3E shows Luciferase imaging of eye (Ey), brain (Br), pituitary gland (Pi), heart (He), thymus (Th), lung (Lu), liver (Li), kidney (Ki), spleen (Sp), pancreas (Pa), testis (Te), adipose (Ad), muscle (Mu), spinal cord (SC), stomach (St), small intestine (In), and cecum(Ce) 2 months after retro-orbital injection of AAV9-UBC-rtTA and AAV9-TRE-Luc followed by treatment with doxycycline for 7 days. The luciferase signal is primarily in liver. Imaging the same tissues with a longer exposure time (FIG. 3E cont.) revealed lower levels of luciferase signal in pancreas (liver was removed).

FIGS. 4A-4B demonstrate viral DNA and luciferase expression in different tissues using single-stranded backbone and various AAV serotypes. All serotypes show large variability of more than 100-fold in DNA load and expression levels between major tissues (See Zincarelli et al 2008). FIG. 4A provides luciferase protein expression profiles of adeno-associated virus (AAV) serotypes 1-9. The levels of luciferase activity [in relative light units (RLU) per mg protein] were determined in selected tissue at 100 days after intravenous injection of 1×10e11 particles of AAV1-9 into adult mice. The data are presented as mean values ±SEM. FIG. 4B provides vector genome copy numbers in selected tissues. Luciferase genome copy numbers/μg of genomic DNA. Persistence of viral genomes in selected tissues 100 days after tail vein injection of 1×10e11particles of adeno-associated virus (AAV) serotypes 1-9. Genomic DNA was isolated from the indicated tissues and 100 ng of each was used in triplicate to determine vector genome copies. Levels of significance were determined using one-way analysis of variance. The data are shown as mean values ±SEM. *P<0.05 versus AAV1, AAV2, AAV3, AAV4, AAV5, AAV6, AAV8. #P<0.05 versus all. **P<0.05 versus all.

FIGS. 5A-5C provide an overview of the DAEUS system. FIG. 5A shows the vector delivery system. FIG. 5B shows AAV DNA biodistribution and FIG. 5C shows GFP expression at a dose of 2e12 vg per mouse using AAV9, PHP.eB or AAV9+PHP.eB together. Note the high tissue-to-tissue variability in viral DNA and GFP expression when AAV9 and PHP.eB are used separately. 18-month old male C57BL6/J mice were injected and euthanized 28 days post-injection. Viral DNA and GFP protein levels were measured via qPCR and Simple Wes respectively.

FIG. 6 shows alanine aminotransferase (ALT) levels 7 days post ssAAV9 (left panel) or scAAV9-miR122 injection (right panel). Elevated ALT levels are indicative of liver damage. Left: elevated ALT levels in ssAAV9-Cisd2 injected mice indicated the need for a strategy of lowering expression in the liver to avoid toxicity. Note no elevation of ALT in ssAAV9-Atg5 injected mice, because Atg5 failed to overexpress with this vector. Right: Addition of miR122 target site to the expression vector reduces ALT increase despite the use of more frail aged mice and more potent vector. Left: 8-week old wild-type male C57BL/6J mice were injected retro-orbitally at a dose of 8e11 vg/mouse (Atg5) and 5e11 vg/mouse (Cisd2). Right: 18-month old wild-type male C57BL/6J mice were retro-orbitally injected with 4e11 vg/mouse of scAAV9-Atg5/Cisd2/GFP. Vehicle used was Final Formulation Buffer. ALT serum levels were measured 7 days post-injection by UMass Mouse Metabolic Phenotyping Center.

FIG. 7 shows scAAV9 vs DAEUS overexpression of Cisd2. AAV9 alone is insufficient to achieve systemic overexpression. Left: 8-week old male C57BL/6J mice were retro-orbitally injected with 4e11 vg/mouse of scAAV9-Cisd2. Right: 18-month old mice were retro-orbitally injected with a total of 4e11 or 2e12 vg/mouse of DAEUS-Cisd2. Mice were euthanized 28 days post-injection and Cisd2 levels measured using Simple Wes.

FIG. 8 shows scAAV9 vs DAEUS overexpression of Atg5. AAV9 alone is insufficient to achieve systemic overexpression. Left: 18 month old male C57BL/6J mice were retro-orbitally injected with 2e12 vg/mouse of scAAV9-Atg5. Right: 18-month old mice were retro-orbitally injected with a total of 8e12 vg/mouse of DAEUS-Atg5. Mice were euthanized 28 days post-injection and Atg5 levels measured using Simple Wes.

FIG. 9 demonstrates DAEUS overexpression of PTEN. 18-month old male and female mice (50:50 ratio) were retro-orbitally injected with a total of 4e11 or 2e12 vg/mouse of DAEUS-PTEN. Mice were euthanized 28 days post-injection and PTEN levels measured using Simple Wes.

FIG. 10 provides dose-response curves of AAV dose to AAV gene transfer for the brain, heart, liver, and tibialis anterior. 5-week old male C57BL6-J mice were injected with doses of approximately 5e12, 2e13, 5e13 and 2e14 AAV vector genomes per kg, at N=3 mice per group with the following serotypes and their combinations: (1) scAAV9-Cbh-GFP-miR122; (2) scAAV9-Cbh-GFP-miRScr (where miRNA target site is scrambled to remove its function); (3) scPHP.eB-Cbh-GFP-miR122; and (4) scPHP.eB-Cbh-GFP-miR122 together with scAAV9-Cbh-GFP-miRScr.

FIG. 11 provides a regression analysis of expected vs observed gene transfer levels. The gene transfer levels observed in the mice of group (1) and group (3) from FIG. 10 were summed for each tissue individually and compared to the observed gene transfer levels in the mice of group (4) of FIG. 10. If no interaction is present between AAV9 and PHP.eB, the sum of gene transfer from groups 1 and 3 for every tissue (Expected) should closely match gene transfer levels in group 4 for every tissue respectively (Observed). The regression analysis of the expected vs observed gene transfer levels indicated that the expected values matched to and correlated highly with the observed values.

FIG. 12 provides a comparison of predicated and observed gene transfer patterns for the brain, heart, liver, and tibialis anterior (TA). 5 groups of 5-week old male C57BL6-J mice were injected retro-orbitally with N=3 mice per group, with different combinations of AAV9 and PHP.eB: (1) 1.4e14 GC/kg scAAV9-Cbh-GFP-miR122+1.9e13 GC/kg scPHP.eB-Cbh-GFP-miR122; (2) 1.9e14 GC/kg scAAV9-Cbh-GFP-miR122+4.8e12 GC/kg scPHP.eB-Cbh-GFP-miR122; (3) 4.8e13 GC/kg scAAV9-Cbh-GFP-miR122+1.9e14 GC/kg scPHP.eB-Cbh-GFP-miR122; (4) 2.4e13 GC/kg scAAV9-Cbh-GFP-miR122+9.5e12 GC/kg scPHP.eB-Cbh-GFP-miR122; and (5) 4.8e12 GC/kg scAAV9-Cbh-GFP-miR122+4.8e13 GC/kg scPHP.eB-Cbh-GFP-miR122. A high match between predicted and observed gene transfer patterns was observed.

FIG. 13 provides a linear regression analysis showing a high correlation of predicted and observed gene transfer levels in the brain, heart, liver, and tibialis anterior (TA) for the different combinations of AAV9 and PHP.eB identified in FIG. 12.

FIG. 14 shows Cisd2 KO mice and their symptoms at 5 months of age. Statistical significance was assessed via two-way ANOVA with Tukey's post-hoc tests.

FIGS. 15A-15D demonstrate effects of DAEUS-Cisd2. Uniform transduction (FIG. 15A) and rescue of Cisd2 expression (FIG. 15B) in Cisd2 knockout Wolfram Syndrome II mice is shown. Rescue of weight (FIG. 15C) and protection against frailty (FIG. 15D) in 2-4 month old Cisd2 knockout mice injected with 4e11 total dose of DAEUS-Cisd2 in shown. Weight was assayed for 155 days post-injection and normalized to weight pre-injection for each mouse. Frailty was assayed 4 months post-injection for Cisd2 knockout mice, Cisd2 knockout mice injected with DAEUS-Cisd2 and their wild-type littermates. Male and female mice were used at approximately 1:1 ratio. Statistical significance was assessed via two-way (left) and one-way (right) ANOVA with Tukey's post-hoc tests.

FIG. 16 shows timelines for assessing effects from administration of DAEUS-Cisd2 on Cisd2 KO mice of various ages (aged (7 months), young (2-4 months), and neonatal (P5-P8)).

FIG. 17 provides results of administering DAEUS-Cisd2 or a vehicle to Cisd2 KO mice aged about P5-P8 days (neonatal) compared to administering a vehicle to WT mice. The data measures survival post-injection, frailty, weight change, speed, and time in movement of mice. The neonatal mice were further observed for corneal scarring or opacity.

FIG. 18 provides results of administering DAEUS-Cisd2 or a vehicle to Cisd2 KO mice aged about 2-4 months (young) compared to administering a vehicle to WT mice. The data measures survival post-injection, frailty, weight change, grid hang ability, and challenging beam crossing of mice.

FIG. 19 provides results of administering DAEUS-Cisd2 or a vehicle to Cisd2 knockout (KO) mice aged about 7 months (aged). Photographs show the mice 40, 64, and 125 days post infection (DPI) and graphs show weight gain and survival of mice who were administered DAEUS-Cisd2 compared to mice that were administered the vehicle (FFB) only. Mice were injected retro-orbitally with a total of 3e11 of DAEUS-Cisd2, then followed for 125 days post-injection (DPI). Vehicle injected mouse died 23 days post-injection.

FIG. 20 shows results of overexpressing DAEUS-PTEN, DAEUS-Atg5, and DAEUS-Cisd2 in WT mice. 18 month old wild-type male and female (1:1 ratio) C57BL6/J mice were injected with either 1e12 vg/mouse of DAEUS-PTEN, 2e12 vg/mouse of DAEUS-Cisd2 or 8e12 vg/mouse of DAEUS-Atg5. Mice were euthanized 1 month post-injection and PTEN, Cisd2 and Atg5 protein levels were measured respectively using Simple Wes. Two separate experiments were performed for each and are shown in individual graphs.

FIG. 21 shows the lifespan of 24 month old wild-type C57BL/6J mice treated with DAEUS-PTEN/Cisd2/GFP or vehicle. Equal numbers of male and female mice were injected retro-orbitally either with vehicle (FFB) (N=14), DAEUS-PTEN at lel2 vg/mouse (PTEN) (N=8), DAEUS-GFP at lel2 vg/mouse (GFP) (N=5) or DAEUS-Cisd2 at 2e12 vg/mouse (Cisd2) (N=6). Survival plotted as with all groups together (top left panel) or individually compared to FFB group (rest). DAEUS-PTEN treated mice showed a 7% increase in overall median survival and 37% increase in post-injection median survival compared to vehicle treated mice. DAEUS-Cisd2 treated mice showed a 7% increase in overall median survival and 38% increase in post-injection median survival compared to FFB treated mice.

### DETAILED DESCRIPTION OF THE INVENTION

Disclosed herein are gene therapy methods that allow for long-term, efficient, and body wide gene expression. Also disclosed herein are viral vector delivery systems for delivery of one or more genes. The viral vector delivery systems described herein deliver genes into the majority of tissues within a subject, provide uniform gene expression across these tissues, provide long-term and stable gene expression, provide strong and efficient expression of the genes so as to achieve overexpression above wild-type levels, and provide evenly distributed gene expression between individual cells. Also disclosed herein are methods of treating or preventing one or more diseases (e.g., Wolfram Syndrome II) or extending the lifespan of a subject by utilizing gene therapy (e.g., a viral vector delivery system) to deliver a gene (e.g., Cisd2, Atg5, of PTEN) to one or more tissues of a subject.

Unless defined otherwise, all technical and scientific terms used herein have the meaning commonly understood by a person skilled in the art. The following references provide one of skill with a general definition of many of the terms used herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3.sup.rd ed., Cold Spring Harbor Laboratory Press, Cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

The present application provides viral vector delivery systems capable of delivering genes to a target environment, for example, a cell, a population of cells, a tissue, an organ, or a combination thereof, in a subject transduced with the viral vector delivery system. For example, the viral vector delivery system can be used to deliver genes to the aorta, endothelium, cardiac muscle, skeletal muscle, tongue, esophagus, stomach, small intestine, large intestine, diaphragm, eye, optic nerve, inner ear, auditory nerve, brown fat, white fat, central nervous system, peripheral nervous system, kidney, spleen, liver, lung, heart, brain, thymus, ovaries, testes, skin, pancreas, bone marrow cells, osteoblasts and osteoclasts, blood cells, hematopoietic stem cells, and muscle satellite cells of a subject. In certain aspects, the viral vector delivery system can be used to deliver genes to the brain, heart, liver, and/or muscle (e.g., transverse abdominal muscle or quadricep muscle) of a subject. Also disclosed herein are peptides capable of directing viral vectors to a target environment (e.g., the brain, the heart, the liver, muscles, or the combination thereof) in a subject, viral vector capsid proteins comprising the peptides, compositions (e.g., pharmaceutical compositions) comprising viral vectors having capsid proteins comprising the peptides, and the nucleic acid sequences encoding the peptides and viral vector capsid proteins. In addition, methods of making and using the viral vectors are also disclosed. In some embodiments, the viral vectors are used to prevent and/or treat one or more diseases and disorders, for example diseases and disorders related to aging.

Disclosed herein are vector delivery systems (e.g., viral vector delivery systems). The viral vector delivery systems may comprise one or more viral serotypes for delivery of a single gene, and in certain aspects may comprise two or more viral serotypes for delivery of a single gene. A viral vector delivery system may comprise an unlimited number of viral serotypes for delivery of a single transgene to a subject. In some embodiments, the viral vector delivery system comprises at least 1, 2, 5, 10, 15, 20, 25, 30, 35, 40, 45, 50, 55, 60, 65, 70, 75, 80, 85, 90, 95, or 100 viral serotypes. In some embodiments, the viral vector delivery system comprises at least one, two, three, four, five, six, seven, eight, nine, or ten viral serotypes. In some embodiments, the viral vector delivery system comprises one to ten, two to eight, five to ten, or five to eight viral serotypes. In some embodiments, the viral vector delivery system comprises one viral serotype. In some embodiments, the viral vector delivery system comprises two viral serotypes. In some embodiments, a first viral serotype delivers a gene to a first target tissue and a second viral serotype delivers the same gene to the first target tissue and/or to a second target tissue. In some aspects, a third, fourth, fifth, sixth, seventh, eighth, ninth, and/or tenth viral serotype delivers the gene to one or more tissues. In some embodiments, the viral serotypes are administered concurrently, proximately, or sequentially.

Suitable viruses for use in the viral vector delivery system described herein include, e.g., adenoviruses, adeno-associated viruses, retroviruses (e.g., lentiviruses), vaccinia virus and other poxviruses, herpesviruses (e.g., herpes simplex virus), and others. The virus may or may not contain sufficient viral genetic information for production of infectious virus when introduced into host cells, i.e., viral vectors may be replication-competent or replication-defective.

In some embodiments, the virus is adeno-associated virus. Adeno-associated virus (AAV) is a small (20 nm) replication-defective, nonenveloped virus. The AAV genome a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA, operably linked to a promoter, is inserted between the inverted terminal repeats (ITR) of the AAV genome. Adeno-associated viruses (AAV) and their use as vectors, e.g., for gene therapy, are also discussed in Snyder, R O and Moullier, P., Adeno-Associated Virus Methods and Protocols, Methods in Molecular Biology, Vol. 807. Humana Press, 2011.

In some embodiments, the virus is AAV serotype 1, 2, 3, 3B, 4, 5, 6, 7, 8, 9, 10, 11, Anc80, or PHP.eB. (disclosed in US 2017/0166926, incorporated herein by reference). Any AAV serotype, or modified AAV serotype, may be used as appropriate and is not limited.

Another suitable AAV may be, e.g., Anc80 (i.e., Anc80 L65) (WO2015054653) or rhlO (WO 2003/042397). Still other AAV sources may include, e.g., PHP.B, PHP.S, hu37 (see, e.g. U.S. Pat. No. 7,906,111; US 2011/0236353), AAV1, AAV2, AAV3, AAV4, AAV5, AAV6, AAV6.2, AAV7, AAV8, (U.S. Pat. Nos. 7,790,449; 7,282,199), AAV9 (U.S. Pat. No. 7,906,111; US 2011/0236353), AAVrh10, AAV-DJ, AAV-DJ/8, AAV.CAP-B10, AAV.CAP-B22, AAVMYO, and others. See, e.g., WO 2003/042397; WO 2005/033321, WO 2006/110689; U.S. Pat. Nos. 7,790,449; 7,282,199; 7,588,772 for sequences of these and other suitable AAV, as well as for methods for generating AAV vectors. Other examples of AAVs include those listed in Table 1. Still other AAVs may be selected, optionally taking into consideration tissue preferences of the selected AAV capsid. In certain embodiments, a viral vector delivery system comprises viral serotypes AAV9 and PHP.eB.

TABLE 1

| Serotype name | Reference |
|---|---|
| AAV1 | ACS Synth. Biol. 8, 194-206 (2019). |
| AAV1A1 | Nat Commun 11, 5432 (2020). |
| AAV1A2 | Nat Commun 11, 5432 (2020). |
| AAV1A6 | Nat Commun 11, 5432 (2020). |
| AAV1P2 | Nat Commun 11, 5432 (2020). |
| AAV1P4 | Nat Commun 11, 5432 (2020). |
| AAV1P5 | Nat Commun 11, 5432 (2020). |
| AAV2 | ACS Synth. Biol. 8, 194-206 (2019). |
| AAV2-7m8 | Sci. Transl. Med. 5, 189ra176 (2013). |
| AAV2A1 | Nat Commun 11, 5432 (2020). |
| AAV2A2 | Nat Commun 11, 5432 (2020). |
| AAV2A6 | Nat Commun 11, 5432 (2020). |
| AAV2-BR1 | EMBO Mol. Med. 8, 609-625 (2016). |
| AAV2-ESGHGYF | Mol. Ther. 24, 1050-1061 (2016). |
| AAV2-ESGHGYFmut1 | Mol. Ther. 24, 1050-1061 (2016). |
| AAV2-ESGHGYFmut2 | Mol. Ther. 24, 1050-1061 (2016). |
| AAV587MTP | Gene Ther. 16, 953-962 (2009) |
| AAV2P2 | Nat Commun 11, 5432 (2020). |
| AAV2P4 | Nat Commun 11, 5432 (2020). |
| AAV2P5 | Nat Commun 11, 5432 (2020). |
| AAV2HBKO | J. Virol. 77, 6995-7006 (2003). |
| AAV2YF | Hum. Gene Ther. 21, 1527-1543 (2010). |
| AAV3b | ACS Synth. Biol. 8, 194-206 (2019). |
| AAV3bA1 | Nat Commun 11, 5432 (2020). |
| AAV3bA2 | Nat Commun 11, 5432 (2020). |
| AAV3bA6 | Nat Commun 11, 5432 (2020). |
| AAV3bP2 | Nat Commun 11, 5432 (2020). |
| AAV3bP4 | Nat Commun 11, 5432 (2020). |
| AAV3bP5 | Nat Commun 11, 5432 (2020). |
| AAV4 | ACS Synth. Biol. 8, 194-206 (2019). |
| AAV4A1 | Nat Commun 11, 5432 (2020). |
| AAV4A2 | Nat Commun 11, 5432 (2020). |
| AAV4A6 | Nat Commun 11, 5432 (2020). |
| AAV4L1 | Nat Commun 11, 5432 (2020). |
| AAV4P2 | Nat Commun 11, 5432 (2020). |
| AAV4P4 | Nat Commun 11, 5432 (2020). |
| AAV4P5 | Nat Commun 11, 5432 (2020). |
| AAV4mut | Nat Commun 11, 5432 (2020). |
| AAV4mutA1 | Nat Commun 11, 5432 (2020). |
| AAV4mutA2 | Nat Commun 11, 5432 (2020). |
| AAV4mutA6 | Nat Commun 11, 5432 (2020). |
| AAV4mutP2 | Nat Commun 11, 5432 (2020). |
| AAV4mutP4 | Nat Commun 11, 5432 (2020). |
| AAV4mutP5 | Nat Commun 11, 5432 (2020). |
| AAV5 | ACS Synth. Biol. 8, 194-206 (2019). |
| AAV5A1 | Nat Commun 11, 5432 (2020). |
| AAV5A2 | Nat Commun 11, 5432 (2020). |
| AAV5A6 | Nat Commun 11, 5432 (2020). |
| AAV5P2 | Nat Commun 11, 5432 (2020). |
| AAV5P4 | Nat Commun 11, 5432 (2020). |
| AAV5P5 | Nat Commun 11, 5432 (2020). |
| AAV6 | ACS Synth. Biol. 8, 194-206 (2019). |
| AAV6A1 | Nat Commun 11, 5432 (2020). |
| AAV6A2 | Nat Common 11, 5432 (2020). |
| AAV6A6 | Nat Commun 11, 5432 (2020). |
| AAV6P2 | Nat Commun 11, 5432 (2020). |
| AAV6P4 | Nat Commun 11, 5432 (2020). |
| AAV6P5 | Nat Commun 11, 5432 (2020). |
| AAV6.2 | Mol. Ther. 17, 294-301 (2009). |
| AAV7 | ACS Synth. Biol. 8, 194-206 (2019). |
| AAV7A1 | Nat Commun 11, 5432 (2020). |
| AAV7A2 | Nat Commun 11, 5432 (2020). |
| AAV7A6 | Nat Commun 11, 5432 (2020). |
| AAV7P2 | Nat Commun 11, 5432 (2020). |
| AAV7P4 | Nat Commun 11, 5432 (2020). |
| AAV7P5 | Nat Commun 11, 5432 (2020). |
| AAV8 | ACS Synth. Biol. 8, 194-206 (2019). |
| AAV8A1 | Nat Commun 11, 5432 (2020). |
| AAV8A2 | Nat Commun 11, 5432 (2020). |
| AAV8A6 | Nat Commun 11, 5432 (2020). |
| AAV8P2 | Nat Commun 11, 5432 (2020). |
| AAV8P4 | Nat Commun 11, 5432 (2020). |
| AAV8P5 | Nat Commun 11, 5432 (2020). |
| AAV9 | ACS Synth. Biol. 8, 194-206 (2019). |
| AAV9A1 | Nat Commun 11, 5432 (2020). |
| AAV9A2 | Nat Commun 11, 5432 (2020). |
| AAV9A6 | Nat Commun 11, 5432 (2020). |
| AAV9BR1 | Nat Commun 11, 5432 (2020). |
| AAV9-SLRSPPS | Gene Ther. 19, 800-809 (2012). |
| AAV9-RGDLRVS | Gene Ther. 19, 800-809 (2012). |
| AAVMYO | Nat Commun 11, 5432 (2020). |
| AAV9P2 | Nat Commun 11, 5432 (2020). |
| AAV9P3 | Nat Commun 11, 5432 (2020). |
| AAV9P4 | Nat Commun 11, 5432 (2020). |
| AAV9P5 | Nat Commun 11, 5432 (2020). |
| AAV-PHP.A | Nat. Biotechnol. 34, 204-209 (2016). |
| AAV-PHP.B | Nat. Biotechnol. 34, 204-209 (2016). |
| AAV-PHP.eB | Nat. Neurosci. 20, 1172-1179 (2017). |
| AAV-PHP.S | Nat. Neurosci. 20, 1172-1179 (2017). |
| AAV9BI | Nat Commun 11, 5432 (2020). |
| AAV9LD | Nat. Commun. 5, 3075 (2014). |
| AAVrh.10 | ACS Synth. Biol. 8, 194-206 (2019). |
| AAVrh.10A1 | Nat Commun 11, 5432 (2020). |
| AAVrh.10A2 | Nat Commun 11, 5432 (2020). |
| AAVrh.10A6 | Nat Commun 11, 5432 (2020). |
| AAVrh.10P2 | Nat Commun 11, 5432 (2020). |
| AAVrh.10P4 | Nat Commun 11, 5432 (2020). |
| AAVrh.10P5 | Nat Commun 11, 5432 (2020). |
| AAVpo.1 | ACS Synth. Biol. 8, 194-206 (2019). |
| AAVpo.1A1 | Nat Commun 11, 5432 (2020). |
| AAVpo.1A2 | Nat Commun 11, 5432 (2020). |
| AAVpo.1A6 | Nat Commun 11, 5432 (2020). |
| AAVpo.1P2 | Nat Commun 11, 5432 (2020). |
| AAVpo.1P4 | Nat Commun 11, 5432 (2020). |
| AAVpo.1P5 | Nat Commun 11, 5432 (2020). |
| AAV12 | ACS Synth. Biol. 8, 194-206 (2019). |
| AAV12A1 | Nat Commun 11, 5432 (2020). |
| AAV12A2 | Nat Commun 11, 5432 (2020). |
| AAV12A6 | Nat Commun 11, 5432 (2020). |
| AAV12P2 | Nat Commun 11, 5432 (2020). |
| AAV12P4 | Nat Commun 11, 5432 (2020). |
| AAV12P5 | Nat Commun 11, 5432 (2020). |
| AAV-Anc80L65 | Cell Reports, 12(6), 1056-1068. |
| AAV-B1 | Mol. Ther. 24, 1247-1257 (2016). |
| AAV-DJ | J. Virol. 82, 5887-5911 (2008). |
| AAV-DJYF | Nat Commun 11, 5432 (2020). |
| AAV-LK03 | Nature 506, 382-386 (2014). |
| AAVM41 | Proc. Natl. Acad. Sci. USA 106, 3946-3951 |
| | (2009). |
| AAV-ShH10 | PLoS One 4, e7467 (2009). |
| AAVAH | Nat Commun 11, 5432 (2020). |
| AAVJEA | Nat Commun 11, 5432 (2020). |
| AAVHSC1 | PLoS One. 2019 Nov. 26; 14(11):e0225582. |
| AAVHSC2 | PLoS One. 2019 Nov. 26; 14(11):e0225582. |
| AAVHSC3 | PLoS One. 2019 Nov. 26; 14(11):e0225582. |
| AAVHSC4 | PLoS One. 2019 Nov. 26; 14(11):e0225582. |
| AAVHSC5 | PLoS One. 2019 Nov. 26; 14(11):e0225582. |
| AAVHSC6 | PLoS One. 2019 Nov. 26; 14(11):e0225582. |
| AAVHSC7 | PLoS One. 2019 Nov. 26; 14(11):e0225582. |
| AAVHSC8 | PLoS One. 2019 Nov. 26; 14(11):e0225582. |
| AAVHSC9 | PLoS One. 2019 Nov. 26; 14(11):e0225582. |
| AAVHSC10 | PLoS One. 2019 Nov. 26; 14(11):e0225582. |
| AAVHSC11 | PLoS One. 2019 Nov. 26; 14(11):e0225582. |
| AAVHSC12 | PLoS One. 2019 Nov. 26; 14(11):e0225582. |
| AAVHSC13 | PLoS One. 2019 Nov. 26; 14(11):e0225582. |
| AAVHSC14 | PLoS One. 2019 Nov. 26; 14(11):e0225582. |
| AAVHSC15 | PLoS One. 2019 Nov. 26; 14(11):e0225582. |
| AAVHSC16 | PLoS One. 2019 Nov. 26; 14(11):e0225582. |
| AAVHSC17 | PLoS One. 2019 Nov. 26; 14(11):e0225582. |
| AAV9-OFF | |
| AAV3B-ON | |
| Anc126 | Cell Reports, 12(6), 1056-1068. |
| Anc127 | Cell Reports, 12(6), 1056-1068. |
| Anc113 | Cell Reports, 12(6), 1056-1068. |
| Anc110 | Cell Reports, 12(6), 1056-1068. |
| Anc81 | Cell Reports, 12(6), 1056-1068. |
| Anc82 | Cell Reports, 12(6), 1056-1068. |
| Anc83 | Cell Reports, 12(6), 1056-1068. |
| Anc84 | Cell Reports, 12(6), 1056-1068. |
| AAV rh32.33 | Journal of Virology November 2009, 83 (24) |
| | 12738-12750 |
| AAV.CAP-B10 | bioRxiv 2020.06.16.152975 |
| AAV.CAP-B1 | bioRxiv 2020.06.16.152975 |
| AAV.CAP-B2 | bioRxiv 2020.06.16.152975 |
| AAV.CAP-B8 | bioRxiv 2020.06.16.152975 |
| AAV.CAP-B10 | bioRxiv 2020.06.16.152975 |
| AAV.CAP-B18 | bioRxiv 2020.06.16.152975 |
| AAV.CAP-B22 | bioRxiv 2020.06.16.152975 |
| AAV2-retro | Sci Rep 10, 6970 (2020). |
| AAV2-QuadYF | Gene Ther 21, 96-105 (2014). |

A recombinant AAV vector (AAV viral particle) may comprise, packaged within an AAV capsid, a nucleic acid molecule containing a 5′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. A shortened version of the 5′ ITR, termed AITR, has been described in which the D-sequence and terminal resolution site (trs) are deleted. The abbreviation “sc” refers to self-complementary. “Self-complementary AAV” refers to a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, ULB, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus- adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless” -containing only the transgene of interest flanked by the signals required for amplification and packaging of the artificial genome), but these genes may be supplied during production.

The one or more viruses may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, the promoter directs expression in a particular cell type (e.g., a targeted population of cells). In some embodiments, the promoter selectively directs expression in any population of cells described herein. In some embodiments, the promoter is a non-silencing promoter. In some embodiments, the promoter is selected from the group consisting chicken β-actin hybrid (Cbh), CAG, CB7, and CBA. In certain embodiments, a non-silencing promoter is Cbh. In some embodiments, the non-silencing promoter directs expression that is high, long-term, and uniform across the cells. For example, the non-silencing promoter, e.g., Cbh, may direct expression that is at least 30%, 40%, 50%, 60%, or 70% of CMV and continues for at least one, two, three, four, five, six, or seven months.

In some embodiments, the viral vector comprises a microRNA (miRNA) target site. In some embodiments, the miRNA target site is engineered into the vector to detarget particular tissues by reducing or silencing expression of the transgene in selected tissues. For example, liver toxicity may be reduced by including a liver-specific miRNA122 target site within the viral vector. In some embodiments, an miRNA target site is selected based on the particular tissues in which expression is to be silenced or reduced. In some embodiments, a viral vector comprises liver specific (e.g., miRNA-33, miRNA-223, miRNA-30c, miRNA-144, miRNA-148a, miRNA-24, miRNA-29, and miRNA-122) (see, e.g., Willeit, et al., Eur Heart J 37, 3260-3266 (2016)), muscle specific (e.g., miRNA-1 and miRNA-133) (see, e.g., Xu et al., J. Cell Sci. 120, 3045-3052 (2007)), cardiac specific (e.g., miRNA-1, miRNA-133, miRNA-208a, miRNA-208b, and miRNA-499) (see, e.g., Xu et al., J. Cell Sci. 120, 3045-3052 (2007), Chistiakov, et al., J. Mol. Cell. Cardiol. 94, 107-121 (2016)), and/or brain specific miRNAs (e.g., miRNA-124 and miRNA-128) (see, e.g., Cao, et al., Genes Dev. 21, 531-536 (2007); Adlakha, et al., Molecular Cancer 13, 33 (2014)). In some embodiments, a viral vector comprises an miRNA target site selected from the group of miRNA-1, miRNA-24, miRNA-29, miRNA-30c, miRNA-33, miRNA-122, miRNA-124, miRNA-128, miRNA-133, miRNA-144, miRNA-148a, miRNA-208a, miRNA-208b, miRNA-223, and miRNA-499. Additional examples of miRNA target sites are available at mirbase.org. See Kozomara A, et al. Nucleic Acids Res 2019 47:D155-D162. In some embodiments, an miRNA target site is an miRNA that is specific (e.g., expressed in a specific tissue at least 10-fold higher than other tissues) and/or highly expressed (e.g., present at levels at least 5X higher than the average levels of all miRNAs in the target tissue). For example, the miRNA can be identified using FANTOM (see De Rie, et al., Nat. Biotechnol. 35, 872-878 (2017)) or other databases known to those of skill in the art.

In some embodiments, a viral vector comprises a self-complementary (self comp) vector backbone. For example, a viral vector may comprise codon-optimized gene coding sequences. In some aspects, a viral vector comprising a self-complementary backbone exhibits increased expression, e.g., at least 2×, 5×, 10×, or 15× greater expression.

In some embodiments, the gene is any gene to be delivered to a tissue. In some embodiments, the gene is associated with a monogenic disease or disorder. In some embodiments, the gene is an aging-related gene or a geroprotective gene. For example, the gene may be any gene listed in Table 2. In some embodiments, the gene is associated with neurological disorders, oncological disorders, retinal disorders, musculoskeletal disorders, hematology/blood disorders, infectious diseases, immunological disorders, etc. Genes may be identified utilizing the OMIM database available at omim.org. In some embodiments, the gene is selected from the group consisting of Cisd2, Atg5, and PTEN.

TABLE 2
From GenAge Database (Nucleic Acids Res. 2018 Jan. 4; 46(D1):D1083-D1090. doi: 10.1093/nar/gkx1042.)

| HGNC Symbol | HAGRID | Common name |
|---|---|---|
| A2M | 139 | alpha-2-macroglobulin |
| ABL1 | 78 | ABL proto-oncogene 1, non-receptor tyrosine kinase |
| ADCY5 | 255 | adenylate cyclase 5 |
| AGPAT2 | 187 | 1-acylglycerol-3-phosphate O-acyltransferase 2 |
| AGTR1 | 264 | angiotensin II receptor, type 1 |
| AIFM1 | 135 | apoptosis-inducing factor, mitochondrion-associated, 1 |
| AKT1 | 35 | v-akt murine thymoma viral oncogene homolog 1 |
| APEX1 | 195 | APEX nuclease (multifunctional DNA repair enzyme) 1 |
| APOC3 | 102 | apolipoprotein C-III |
| APOE | 138 | apolipoprotein E |
| APP | 137 | amyloid beta (A4) precursor protein |
| APTX | 95 | aprataxin |
| AR | 110 | androgen receptor |
| ARHGAP1 | 249 | Rho GTPase activating protein 1 |
| ARNTL | 251 | aryl hydrocarbon receptor nuclear translocator-like |
| ATF2 | 193 | activating transcription factor 2 |
| ATM | 9 | ATM serine/threonine kinase |
| ATP5O | 146 | ATP synthase, H+ transporting, mitochondrial F1 complex, O subunit |
| ATR | 231 | ATR serine/threonine kinase |
| BAK1 | 278 | BCL2-antagonist/killer 1 |
| BAX | 119 | BCL2-associated X protein |
| BCL2 | 69 | B-cell CLL/lymphoma 2 |
| BDNF | 191 | brain-derived neurotrophic factor |
| BLM | 68 | Bloom syndrome, RecQ helicase-like |
| BMI1 | 188 | BMI1 proto-oncogene, polycomb ring finger |
| BRCA1 | 61 | breast cancer 1, early onset |
| BRCA2 | 79 | breast cancer 2, early onset |
| BSCL2 | 186 | Berardinelli-Seip congenital lipodystrophy 2 (seipin) |
| BUB1B | 197 | BUB1 mitotic checkpoint serine/threonine kinase B |
| BUB3 | 240 | BUB3 mitotic checkpoint protein |
| C1QA | 287 | complement component 1, q subcomponent, A chain |
| CACNA1A | 133 | calcium channel, voltage-dependent, P/Q type, alpha 1A subunit |
| CAT | 107 | catalase |
| CCNA2 | 177 | cyclin A2 |
| CDC42 | 250 | cell division cycle 42 |
| CDK1 | 201 | cyclin-dependent kinase 1 |
| CDK7 | 299 | cyclin-dependent kinase 7 |
| CDKN1A | 284 | cyclin-dependent kinase inhibitor 1A (p21, Cip1) |
| CDKN2A | 226 | cyclin-dependent kinase inhibitor 2A |
| CDKN2B | 288 | cyclin-dependent kinase inhibitor 2B (p15, inhibits CDK4) |
| CEBPA | 88 | CCAAT/enhancer binding protein (C/EBP), alpha |
| CEBPB | 89 | CCAAT/enhancer binding protein (C/EBP), beta |
| CETP | 262 | cholesteryl ester transfer protein, plasma |
| CHEK2 | 247 | checkpoint kinase 2 |
| CISD2 | 265 | CDGSH iron sulfur domain 2 |
| CLOCK | 252 | clock circadian regulator |
| CLU | 220 | clusterin |
| CNR1 | 282 | cannabinoid receptor 1 (brain) |
| COQ7 | 132 | coenzyme Q7 homolog, ubiquinone (yeast) |
| CREB1 | 192 | cAMP responsive element binding protein 1 |
| CREBBP | 64 | CREB binding protein |
| CSNK1E | 244 | casein kinase 1, epsilon |
| CTF1 | 304 | cardiotrophin 1 |
| CTGF | 206 | connective tissue growth factor |
| CTNNB1 | 223 | catenin (cadherin-associated protein), beta 1, 88 kDa |
| DBN1 | 228 | drebrin 1 |
| DDIT3 | 203 | DNA-damage-inducible transcript 3 |
| DGAT1 | 291 | diacylglycerol O-acyltransferase 1 |
| DLL3 | 225 | delta-like 3 (Drosophila) |
| E2F1 | 18 | E2F transcription factor 1 |
| EEF1A1 | 189 | eukaryotic translation elongation factor 1 alpha 1 |
| EEF1E1 | 266 | eukaryotic translation elongation factor 1 epsilon 1 |
| EEF2 | 103 | eukaryotic translation elongation factor 2 |
| EFEMP1 | 260 | EGF containing fibulin-like extracellular matrix protein 1 |
| EGF | 48 | epidermal growth factor |
| EGFR | 40 | epidermal growth factor receptor |
| EGR1 | 76 | early growth response 1 |
| EIF5A2 | 289 | eukaryotic translation initiation factor 5A2 |
| ELN | 230 | elastin |
| EMD | 121 | emerin |
| EP300 | 94 | E1A binding protein p300 |
| EPOR | 52 | Erythropoietin receptor |
| EPS8 | 267 | epidermal growth factor receptor pathway substrate 8 |
| ERBB2 | 41 | erb-b2 receptor tyrosine kinase 2 |
| ERCC1 | 149 | excision repair cross-complementation group 1 |
| ERCC2 | 11 | excision repair cross-complementation group 2 |
| ERCC3 | 104 | excision repair cross-complementation group 3 |
| ERCC4 | 261 | excision repair cross-complementation group 4 |
| ERCC5 | 109 | excision repair cross-complementation group 5 |
| ERCC6 | 92 | excision repair cross-complementation group 6 |
| ERCC8 | 12 | excision repair cross-complementation group 8 |
| ESR1 | 216 | estrogen receptor 1 |
| FAS | 115 | Fas cell surface death receptor |
| FEN1 | 114 | flap structure-specific endonuclease 1 |
| FGF21 | 293 | fibroblast growth factor 21 |
| FGF23 | 259 | fibroblast growth factor 23 |
| FGFR1 | 169 | fibroblast growth factor receptor 1 |
| FLT1 | 171 | fms-related tyrosine kinase 1 |
| FOS | 50 | FBJ murine osteosarcoma viral oncogene homolog |
| FOXM1 | 131 | forkhead box M1 |
| FOXO1 | 124 | forkhead box O1 |
| FOXO3 | 123 | forkhead box O3 |
| FOXO4 | 183 | forkhead box O4 |
| GCLC | 237 | glutamate-cysteine ligase, catalytic subunit |
| GCLM | 238 | glutamate-cysteine ligase, modifier subunit |
| GDF11 | 309 | growth differentiation factor 11 |
| GH1 | 26 | growth hormone 1 |
| GHR | 1 | growth hormone receptor |
| GHRH | 2 | growth hormone releasing hormone |
| GHRHR | 222 | growth hormone releasing hormone receptor |
| GPX1 | 153 | glutathione peroxidase 1 |
| GPX4 | 257 | glutathione peroxidase 4 |
| GRB2 | 122 | growth factor receptor-bound protein 2 |
| GRN | 300 | granulin |
| GSK3A | 295 | glycogen synthase kinase 3 alpha |
| GSK3B | 97 | glycogen synthase kinase 3 beta |
| GSR | 154 | glutathione reductase |
| GSS | 155 | glutathione synthetase |
| GSTA4 | 156 | glutathione S-transferase alpha 4 |
| GSTP1 | 157 | glutathione S-transferase pi 1 |
| GTF2H2 | 111 | general transcription factor IIH, polypeptide 2, 44 kDa |
| H2AFX | 212 | H2A histone family, member X |
| HBP1 | 196 | HMG-box transcription factor 1 |
| HDAC1 | 151 | histone deacetylase 1 |
| HDAC2 | 207 | histone deacetylase 2 |
| HDAC3 | 25 | histone deacetylase 3 |
| HELLS | 101 | helicase, lymphoid-specific |
| HESX1 | 184 | HESX homeobox 1 |
| HIC1 | 253 | hypermethylated in cancer 1 |
| HIF1A | 65 | hypoxia inducible factor 1, alpha subunit (basic helix-loop-helix transcription factor) |
| HMGB1 | 176 | high mobility group box 1 |
| HMGB2 | 178 | high mobility group box 2 |
| HOXB7 | 213 | homeobox B7 |
| HOXC4 | 214 | homeobox C4 |
| HRAS | 38 | Harvey rat sarcoma viral oncogene homolog |
| HSF1 | 125 | heat shock transcription factor 1 |
| HSP90AA1 | 74 | heat shock protein 90 kDa alpha (cytosolic), class A member 1 |
| HSPA1A | 160 | heat shock 70 kDa protein 1A |
| HSPA1B | 161 | heat shock 70 kDa protein 1B |
| HSPA8 | 199 | heat shock 70 kDa protein 8 |
| HSPA9 | 152 | heat shock 70 kDa protein 9 (mortalin) |
| HSPD1 | 159 | heat shock 60 kDa protein 1 (chaperonin) |
| HTRA2 | 294 | HtrA serine peptidase 2 |
| HTT | 98 | huntingtin |
| IFNB1 | 308 | Interferon beta |
| IGF1 | 28 | insulin-like growth factor 1 (somatomedin C) |
| IGF1R | 15 | insulin-like growth factor 1 receptor |
| IGF2 | 29 | insulin-like growth factor 2 |
| IGFBP2 | 279 | insulin-like growth factor binding protein 2, 36 kDa |
| IGFBP3 | 73 | insulin-like growth factor binding protein 3 |
| IKBKB | 297 | inhibitor of kappa light polypeptide gene enhancer in B-cells, kinase beta |
| IL2 | 46 | interleukin 2 |
| IL2RG | 49 | interleukin 2 receptor, gamma |
| IL6 | 144 | interleukin 6 |
| IL7 | 167 | interleukin 7 |
| IL7R | 27 | interleukin 7 receptor |
| INS | 30 | insulin |
| INSR | 42 | insulin receptor |
| IRS1 | 32 | insulin receptor substrate 1 |
| IRS2 | 34 | insulin receptor substrate 2 |
| JAK2 | 215 | Janus kinase 2 |
| JUN | 172 | jun proto-oncogene |
| JUND | 45 | jun D proto-oncogene |
| KCNA3 | 268 | potassium channel, voltage gated shaker related subfamily A, member 3 |
| KL | 17 | klotho |
| LEP | 217 | leptin |
| LEPR | 218 | leptin receptor |
| LMNA | 14 | lamin A/C |
| LMNB1 | 181 | lamin B1 |
| LRP2 | 134 | low density lipoprotein receptor-related protein 2 |
| MAP3K5 | 179 | mitogen-activated protein kinase kinase kinase 5 |
| MAPK14 | 168 | mitogen-activated protein kinase 14 |
| MAPK3 | 175 | mitogen-activated protein kinase 3 |
| MAPK8 | 163 | mitogen-activated protein kinase 8 |
| MAPK9 | 174 | mitogen-activated protein kinase 9 |
| MAPT | 205 | microtubule-associated protein tau |
| MAX | 208 | MYC associated factor X |
| MDM2 | 210 | MDM2 proto-oncogene, E3 ubiquitin protein ligase |
| MED1 | 173 | mediator complex subunit 1 |
| MIF | 290 | macrophage migration inhibitory factor (glycosylation-inhibiting factor) |
| MLH1 | 243 | mutL homolog 1 |
| MSRA | 127 | methionine sulfoxide reductase A |
| MT-CO1 | 158 | mitochondrially encoded cytochrome c oxidase I |
| MT1E | 292 | metallothionein 1E |
| MTOR | 221 | mechanistic target of rapamycin (serine/threonine kinase) |
| MXD1 | 209 | MAX dimerization protein 1 |
| MXI1 | 90 | MAX interactor 1, dimerization protein |
| MYC | 39 | v-myc avian myelocytomatosis viral oncogene homolog |
| NBN | 44 | nibrin |
| NCOR1 | 43 | nuclear receptor corepressor 1 |
| NCOR2 | 276 | nuclear receptor corepressor 2 |
| NFE2L1 | 307 | nuclear factor, erythroid 2-like 1 |
| NFE2L2 | 283 | nuclear factor, erythroid 2-like 2 |
| NFKB1 | 82 | nuclear factor of kappa light polypeptide gene enhancer in B-cells 1 |
| NFKB2 | 20 | nuclear factor of kappa light polypeptide gene enhancer in B-cells 2 (p49/p100) |
| NFKBIA | 219 | nuclear factor of kappa light polypeptide gene enhancer in B-cells inhibitor, alpha |
| NGF | 31 | nerve growth factor (beta polypeptide) |
| NGFR | 37 | nerve growth factor receptor |
| NOG | 229 | noggin |
| NR3C1 | 75 | nuclear receptor subfamily 3, group C, member 1 (glucocorticoid receptor) |
| NRG1 | 24 | neuregulin 1 |
| NUDT1 | 296 | nudix (nucleoside diphosphate linked moiety X)-type motif 1 |
| PAPPA | 254 | pregnancy-associated plasma protein A, pappalysin 1 |
| PARP1 | 60 | poly (ADP-ribose) polymerase 1 |
| PCK1 | 248 | phosphoenolpyruvate carboxykinase 1 (soluble) |
| PCMT1 | 162 | protein-L-isoaspartate (D-aspartate) O-methyltransferase |
| PCNA | 113 | proliferating cell nuclear antigen |
| PDGFB | 47 | platelet-derived growth factor beta polypeptide |
| PDGFRA | 285 | platelet-derived growth factor receptor, alpha polypeptide |
| PDGFRB | 51 | platelet-derived growth factor receptor, beta polypeptide |
| PDPK1 | 87 | 3-phosphoinositide dependent protein kinase 1 |
| PEX5 | 58 | peroxisomal biogenesis factor 5 |
| PIK3CA | 286 | phosphatidylinositol-4,5-bisphosphate 3-kinase, catalytic subunit alpha |
| PIK3CB | 36 | phosphatidylinositol-4,5-bisphosphate 3-kinase, catalytic subunit beta |
| PIK3R1 | 185 | phosphoinositide-3-kinase, regulatory subunit 1 (alpha) |
| PIN1 | 62 | peptidylprolyl cis/trans isomerase, NIMA-interacting 1 |
| PLAU | 10 | plasminogen activator, urokinase |
| PLCG2 | 57 | phospholipase C, gamma 2 (phosphatidylinositol-specific) |
| PMCH | 242 | pro-melanin-concentrating hormone |
| PML | 96 | promyelocytic leukemia |
| POLA1 | 204 | polymerase (DNA directed), alpha 1, catalytic subunit |
| POLB | 236 | polymerase (DNA directed), beta |
| POLD1 | 118 | polymerase (DNA directed), delta 1, catalytic subunit |
| POLG | 72 | polymerase (DNA directed), gamma |
| PON1 | 142 | paraoxonase 1 |
| POU1F1 | 4 | POU class 1 homeobox 1 |
| PPARA | 55 | peroxisome proliferator-activated receptor alpha |
| PPARG | 263 | peroxisome proliferator-activated receptor gamma |
| PPARGC1A | 256 | peroxisome proliferator-activated receptor gamma, coactivator 1 alpha |
| PPM1D | 246 | protein phosphatase, Mg2+/Mn2+ dependent, 1D |
| PPP1CA | 227 | protein phosphatase 1, catalytic subunit, alpha isozyme |
| PRDX1 | 141 | peroxiredoxin 1 |
| PRKCA | 99 | protein kinase C, alpha |
| PRKCD | 54 | protein kinase C, delta |
| PRKDC | 106 | protein kinase, DNA-activated, catalytic polypeptide |
| PROP1 | 5 | PROP paired-like homeobox 1 |
| PSEN1 | 224 | presenilin 1 |
| PTEN | 63 | phosphatase and tensin homolog |
| PTGS2 | 198 | prostaglandin-endoperoxide synthase 2 (prostaglandin G/H synthase and cyclooxygenase) |
| PTK2 | 166 | protein tyrosine kinase 2 |
| PTK2B | 165 | protein tyrosine kinase 2 beta |
| PTPN1 | 33 | protein tyrosine phosphatase, non-receptor type 1 |
| PTPN11 | 19 | protein tyrosine phosphatase, non-receptor type 11 |
| PYCR1 | 280 | pyrroline-5-carboxylate reductase 1 |
| RAD51 | 84 | RAD51 recombinase |
| RAD52 | 147 | RAD52 homolog, DNA repair protein |
| RAE1 | 241 | ribonucleic acid export 1 |
| RB1 | 120 | retinoblastoma 1 |
| RECQL4 | 128 | RecQ helicase-like 4 |
| RELA | 143 | v-rel avian reticuloendotheliosis viral oncogene homolog A |
| RET | 56 | ret proto-oncogene |
| RGN | 145 | regucalcin |
| RICTOR | 303 | RPTOR independent companion of MTOR, complex 2 |
| RPA1 | 67 | replication protein A1, 70 kDa |
| S100B | 70 | S100 calcium binding protein B |
| SDHC | 182 | succinate dehydrogenase complex, subunit C, integral membrane protein, 15 kDa |
| SERPINE1 | 301 | serpin peptidase inhibitor, clade E (nexin, plasminogen activator inhibitor type 1), member 1 |
| SHC1 | 3 | SHC (Src homology 2 domain containing) transforming protein 1 |
| SIN3A | 200 | SIN3 transcription regulator family member A |
| SIRT1 | 150 | sirtuin 1 |
| SIRT3 | 275 | sirtuin 3 |
| SIRT6 | 239 | sirtuin 6 |
| SIRT7 | 269 | sirtuin 7 |
| SLC13A1 | 270 | solute carrier family 13 (sodium/sulfate symporter), member 1 |
| SNCG | 140 | synuclein, gamma (breast cancer-specific protein 1) |
| SOCS2 | 271 | suppressor of cytokine signaling 2 |
| SOD1 | 130 | superoxide dismutase 1, soluble |
| SOD2 | 129 | superoxide dismutase 2, mitochondrial |
| SP1 | 170 | Sp1 transcription factor |
| SPRTN | 302 | SprT-like N-terminal domain |
| SQSTM1 | 298 | sequestosome 1 |
| SST | 53 | somatostatin |
| SSTR3 | 100 | somatostatin receptor 3 |
| STAT3 | 22 | signal transducer and activator of transcription 3 (acute-phase response factor) |
| STAT5A | 23 | signal transducer and activator of transcription 5A |
| STAT5B | 21 | signal transducer and activator of transcription 5B |
| STK11 | 93 | serine/threonine kinase 11 |
| STUB1 | 245 | STIP1 homology and U-box containing protein 1, E3 ubiquitin protein ligase |
| SUMO1 | 211 | small ubiquitin-like modifier 1 |
| SUN1 | 277 | Sadi and UNC84 domain containing 1 |
| TAF1 | 180 | TAF1 RNA polymerase II, TATA box binding protein (TBP)-associated factor, 250 kDa |
| TBP | 194 | TATA box binding protein |
| TCF3 | 59 | transcription factor 3 |
| TERC | 7 | telomerase RNA component |
| TERF1 | 105 | telomeric repeat binding factor (NIMA-interacting) 1 |
| TERF2 | 116 | telomeric repeat binding factor 2 |
| TERT | 8 | telomerase reverse transcriptase |
| TFAP2A | 190 | transcription factor AP-2 alpha (activating enhancer binding protein 2 alpha) |
| TFDP1 | 202 | transcription factor Dp-1 |
| TGFB1 | 91 | transforming growth factor, beta 1 |
| TNF | 86 | tumor necrosis factor |
| TOP1 | 83 | topoisomerase (DNA) I |
| TOP2A | 80 | topoisomerase (DNA) II alpha |
| TOP2B | 81 | topoisomerase (DNA) II beta |
| TOP3B | 148 | topoisomerase (DNA) III beta |
| TP53 | 6 | tumor protein p53 |
| TP53BP1 | 274 | tumor protein p53 binding protein 1 |
| TP63 | 234 | tumor protein p63 |
| TP73 | 281 | tumor protein p73 |
| TPP2 | 273 | tripeptidyl peptidase II |
| TRAP1 | 305 | TNF receptor-associated protein 1 |
| TRPV1 | 306 | transient receptor potential cation channel subfamily V member 1 |
| TXN | 16 | thioredoxin |
| UBB | 66 | ubiquitin B |
| UBE2I | 85 | ubiquitin-conjugating enzyme E2I |
| UCHL1 | 136 | ubiquitin carboxyl-terminal esterase L1 (ubiquitin thiolesterase) |
| UCP1 | 258 | uncoupling protein 1 (mitochondrial, proton carrier) |
| UCP2 | 235 | uncoupling protein 2 (mitochondrial, proton carrier) |
| UCP3 | 232 | uncoupling protein 3 (mitochondrial, proton carrier) |
| VCP | 71 | valosin containing protein |
| VEGFA | 77 | vascular endothelial growth factor A |
| WRN | 13 | Werner syndrome, RecQ helicase-like |
| XPA | 126 | xeroderma pigmentosum, complementation group A |
| XRCC5 | 112 | X-ray repair complementing defective repair in Chinese hamster cells 5 (double-strand-break rejoining) |
| XRCC6 | 117 | X-ray repair complementing defective repair in Chinese hamster cells 6 |
| YWHAZ | 164 | tyrosine 3-monooxygenase/tryptophan 5-monooxygenase activation protein, zeta |
| ZMPSTE24 | 233 | zinc metallopeptidase STE24 |
| Dsup | | Tardigrade radiation resistance DNA repair protein |

In some embodiments, a viral vector delivery system comprises an AAV9 serotype and/or a PHP.eB serotype for delivery of the Cisd2 gene to a subject. In some embodiments, the viral vector delivery system comprises a miRNA target site, e.g., a miRNA-122 target site. In some embodiments, the viral vector delivery system comprises a non-silencing promoter, e.g., Cbh, and optionally further comprises a self-complementary backbone.

The viral vector delivery system may result in overexpression of a native gene by at least 10%, 15%, 20%, 25%, 30%, 35%, 40%, 45%, 50%, 55%, 60%, 65%, 70%, or 75% of wild-type levels in a target tissue (e.g., in at least 70% of fat free, blood free body mass). In some embodiments, the viral vector delivery system may result in overexpression of a native gene by at least 100%, 200%, 300%, 400%, 500%, 600%, 700%, 800%, 900%, 1000%, 1500%, 2000%, 2500%, 5000%, 7500%, 10000%, 50000%, 100000% of wild-type levels in a target tissue. In some embodiments, the viral vector delivery system delivers a native gene resulting in overexpression of the native gene by about 10%-90%, 20%-80%, 30%-70%, or 40%-60% of wild-type levels in a tissue. In some embodiments, the viral vector delivery system results in overexpression of a native gene by at least 30%, or by about 25-50%, of wild-type levels. The viral vector delivery system may result in detectable expression (e.g., greater than trace expression) of a non-native gene in a target tissue (e.g., in at least 70% of fat free, blood free body mass). In some embodiments, expression of the delivered gene is stable and long-term (e.g., expression is maintained for at least 1 month, 2 months, 3 months, 4 months, 5 months, 6 months, 7 months, 8 months, 9 months, 10 months, 11 months, 12 months, 15 months, 18 months, 21 months, 24 months, 3 years, 4 years, 5 years, 10 years, 15 years, 20 years, 30 years, 40 years, 50 years, 60 years, 70 years, 80 years, 90 years).

In some embodiments, the viral vector delivery system delivers a gene of interest to a tissue of interest (e.g., aorta, endothelium, cardiac muscle skeletal muscle, tongue, esophagus, stomach, small intestine, large intestine, diaphragm, eye, optic nerve, inner ear, auditory nerve, brown fat, white fat, central nervous system, peripheral nervous system, kidney, spleen, liver, lung, heart, brain, thymus, ovaries, testes, skin, pancreas, bone marrow cells, osteoblasts and osteoclasts, blood cells, hematopoietic stem cells, and/or muscle satellite cells). In some embodiments, the viral vector delivery system delivers a gene of interest to multiple tissues of interest in a subject. For example, the viral vector delivery system may deliver a gene of interest to at least 20%, 30%, 40%, 50%, 60%, 70%, 80%, or 90% of tissues in a subject. In some embodiments, the viral vector delivery system delivers a gene to about 10%-90%, 20%-80%, 30%-70%, or 40%-60% of tissues in the subject. The viral vector delivery system may provide uniform or limited variable delivery of a gene across multiple tissues within a subject.

Some embodiments of the present invention relate to methods of treatment or prevention for a disease or condition, such as an aging-related disease or disorder, by the delivery of a pharmaceutical composition comprising an effective amount of the viral vector delivery system described herein. An effective amount of the pharmaceutical composition is an amount sufficient to prevent, slow, inhibit, or ameliorate a disease or disorder in a subject to whom the composition is administered. In some embodiments, the delivery of a pharmaceutical composition comprising an effective amount of the viral vector delivery system described herein extends the life expectancy or lifespan of a subject.

In some embodiments, the viral vector delivery system is administered to a subject. The viral vector delivery system may deliver a gene to a subject, e.g., to one or more tissues of a subject. In some embodiments, the subject is expected to suffer from a disease or disorder based on family history or genetic analysis but is not currently suffering from the disease or disorder. In some embodiments, the subject is suffering from a disease or disorder. In some embodiments, the subject lacks an effective amount of active Cisd2. For example, the Cisd2 gene may be mutated or otherwise inactive in a subject. The gene may be delivered using the viral vector delivery system to treat or ameliorate the disease or disorder in the subject.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein. Preferably, the subject is a mammal. The mammal can be a human, non-human primate, mouse, rat, dog, cat, horse, or cow, but are not limited to these examples. In addition, the methods described herein can be used to treat domesticated animals and/or pets. A subject can be male or female. A subject can be one who has been previously diagnosed with or identified as suffering from or having a condition in need of treatment or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors for a condition or one or more complications related to a condition. A “subject in need” of treatment for a particular condition can be a subject having that condition, diagnosed as having that condition, or at increased risk of developing that condition relative to a given reference population.

As used herein, “treat,” “treatment,” “treating,” or “amelioration” when used in reference to a disease, disorder or medical condition, refer to therapeutic treatments for a condition, wherein the object is to prevent, reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state as compared to that expected in the absence of treatment.

In some embodiments, the viral vector delivery system is administered for immunological purposes, e.g., for vaccination or tolerance induction.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

In accordance with methods of the invention, treatment comprises contacting one or more tissues with a composition according to the invention. The routes of administration will vary and include, e.g., intradermal, transdermal, parenteral, intravenous, intramuscular, intranasal, subcutaneous, regional, percutaneous, intratracheal, intraperitoneal, intraarterial, intravesical, intraocular, intratumoral, inhalation, perfusion, lavage, and oral administration and formulation. Treatment regimens may vary as well, and often depend on disease type, disease location, disease progression, and health and age of the patient.

The treatments may include various “unit doses” defined as containing a predetermined-quantity of the therapeutic composition. The quantity to be administered, and the particular route and formulation, are within the skill of those in the clinical arts. A unit dose need not be administered as a single injection but may comprise continuous infusion over a specified period of time. The dosage ranges for the agent depends upon the potency, and are amounts large enough to produce the desired effect. The dosage should not be so large as to cause unacceptable adverse side effects.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

The pharmaceutical compositions disclosed herein may be administered intratumorally, parenterally, intravenously, intradermally, intramuscularly, transdermally or even intraperitoneally as described in U.S. Pat. Nos. 5,543,158; 5,641,515 and 5,399,363.

Injection of the viral vector delivery system may be delivered by syringe or any other method used for injection of a solution, as long as the expression construct can pass through the particular gauge of needle required for injection and the dosage can be administered with the required level of precision.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The phrase “pharmaceutically-acceptable” or “pharmacologically-acceptable” refers to molecular entities and compositions that do not produce an allergic or similar untoward reaction when administered to a subject. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Except insofar as any conventional media or agent is incompatible with the viral agent, its use in the therapeutic compositions is contemplated. Supplementary active ingredients can also be incorporated into the compositions.

In some embodiments, the methods further comprise administering the pharmaceutical composition described herein along with one or more additional agents, biologics, drugs, or treatments beneficial to a subject suffering from a disorder or disease.

In some embodiments, the viral vector delivery system or pharmaceutical compositions comprising the viral vector delivery system are administered to a subject to treat a disease or condition. The disease or condition may be an aging-related disease or condition. In some embodiments, the disease or condition is a progeria syndrome, (e.g., Hutchinson-Gilford progeria syndrome (HGPS), Wolfram Syndrome (e.g., Wolfram Syndrome I or II), Werner Syndrome, Cockayne syndrome, Myotonic Dystrophy type 1, MDPL syndrome, Dyskeratosis congenital disorder, etc.), connective tissue disorder (e.g., Marfan syndrome, Loeys-Dietz syndrome, Ehlers-Danlos syndrome, Osteogenesis Imperfecta, etc.), metabolic disorders (e.g., Methylmalonic Acidemia, Wilson's disease, etc.), tumor suppressor and DNA replication deficiency disorders (e.g., PTENopathies (Cowden syndrome, Proteus-like syndromes), Bloom syndrome, RASopathies (Noonan syndrome, Costello syndrome)), neurodegenerative disorder (e.g., Alzheimer's disease, dementia, mild cognitive decline, etc.), neurovascular disorder (e.g., stroke), skeletal muscle conditions (e.g., sarcopenia, frailty), Cowden syndrome, Bannayan-Riley-Ruvalcaba syndrome, Proteus syndrome, Proteus-like syndrome and other PTEN-opathies. Werner syndrome, Bloom syndrome, Rothmund-Thomson syndrome, Cockayne syndrome, xeroderma pigmentosum, trichothiodystrophy, combined xeroderma pigmentosum-Cockayne syndrome, restrictive dermopathy, diabetes, obesity, cardiovascular disease, cancer, ocular degeneration, liver failure, and age-related macular degeneration. See Schnabel, F., et al., Premature aging disorders: A clinical and genetic compendium. Clinical Genetics 99,3-28 (2020); Rigoli, L,. et al., Wolfram syndrome 1 and Wolfram syndrome 2. Curr. Opin. Pediatr. 24,1 (2012); Keane, M. G., et al., Medical management of marfan syndrome. Circulation 117, 2802-2813 (2008); MacCarrick, G. et al., Loeys-Dietz syndrome: A primer for diagnosis and management. Genet. Med. 16,576-587 (2014); Mao, J. R. et al., The Ehlers-Danlos syndrome: On beyond collagens. Journal of Clinical Investigation 107, 1063-1069 (2001); van Dijk, F. S. et al., Osteogenesis Imperfecta: A Review with Clinical Examples. Mol. Syndromol. 2,1-20 (2011); Yehia, L., et al., PTEN-opathies: from biological insights to evidence-based precision medicine. J. Clin. Invest. 129, 452-464 (2019); Cunniff, C., et al., Bloom's Syndrome: Clinical Spectrum, Molecular Pathogenesis, and Cancer Predisposition. Mol. Syndromol. 8,4-23 (2017); and Rauen, K. A. The RASopathies. Annu. Rev. Genomics Hum. Genet. 14,355-369 (2013). The subject may be suffering from any disease or condition that would benefit from administration of a gene to two or more types of tissue.

In some embodiments, the neurodegenerative disorder is one of polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In some embodiments, the neurovascular disorder is selected from the group consisting of brain atherothrombosis, brain aneurysms, brain arteriovenous malformations, brain embolism, brain ischemia, for example caused by atherothrombosis, embolism, or hemodynamic abnormalities, cardiac arrest, carotid stenosis, cerebrovascular spasm, headache, intracranial hemorrhage, ischemic stroke, seizure, spinal vascular malformations, reflex neurovascular dystrophy (RND), neurovascular compression disorders such as hemifacial spasms, tinnitus, trigeminal neuralgia, glossopharyngeal neuralgia, stroke, transient ischemic attacks, and vasculitis.

In some embodiments, the skeletal muscle condition is selected from the group consisting of atrophy, bony fractures associated with muscle wasting or weakness, cachexia, denervation, diabetes, dystrophy, exercise-induced skeletal muscle fatigue, fatigue, frailty, inflammatory myositis, metabolic syndrome, neuromuscular disease, obesity, post-surgical muscle weakness, post-traumatic muscle weakness, sarcopenia, toxin exposure, wasting, and weakness.

In some embodiments, a vector delivery system or a pharmaceutical composition comprising the vector delivery system is administered (e.g., intravenously) to a subject. The vector delivery system may deliver a gene, e.g., Cisd2, to the subject to treat a disease or condition associated with mutated Cisd2 (e.g., Wolfram Syndrome II or related condition, i.e., loss of vision or cataracts, diabetes, deafness, kidney failure, etc.).

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXEMPLIFICATION

### Example 1

Aging is the single biggest factor in the most burdensome diseases today. The major concerns of current health-care systems are diabetes, obesity, cardiovascular disease, cancers, frailty, age-related macular degeneration and neurodegenerative diseases. All of them share advanced age as the common and largest risk factor. [1] The dominant paradigm in medicine now is to treat these diseases individually after they appear and continue managing the symptoms through multiple treatment modalities. However, this is a downhill battle, as most aged patients never recover and instead develop further morbidities with advanced age, all of which require chronic treatment. The average patient is now being treated for decades for multiple chronic diseases, at a cost that is often considerably higher than that patient's lifetime contribution to the healthcare system. [2] This is non-sustainable.

From fundamental studies into the biology of aging, over 50 genes which influence lifespan in the mouse have been identified. [3] Based on these findings, a new class of drugs terms geroprotectors has emerged, which target conserved aging pathways (e.g., proteostasis, autophagy, insulin-IGF signaling, mitochondrial metabolism and other pathways) at a systemic level. They have gone through an explosive growth in number and investment over the past 5 years, with over 200 different drugs now existing. [4]

These drugs are important first steps towards preventing age-related diseases at their source and will likely go on to have a large patient population. However, small-molecule drugs are fundamentally limited as geroprotectors due to three aspects. Firstly, they have side-effects. Side-effects are caused by off-target effects and on-target effects in tissues where perturbation of the target is unwanted. While side-effects are tolerated for other drugs, these drugs are expected to treat healthy people, and will thus have to have very mild side-effects (if at all) to justify their usage. Secondly, they require continuous, life-long administration. While this may be possible for cheap drugs such as metformin, for many others this is prohibitively costly or cumbersome (e.g., for drugs that require injections). Finally, these drugs can only achieve limited efficacy, as they cannot perturb the function of their targets as fully as is possible via genetic methods.

Therefore, successful therapies that aim to prevent age-related diseases need to be long-acting, tissue-specific, and be able to perturb intracellular networks precisely and completely. This is not achievable with small-molecule drugs or biologics but is achievable with gene therapies. In fact, gene therapy seems to be the only viable method in the long term that meets these requirements.

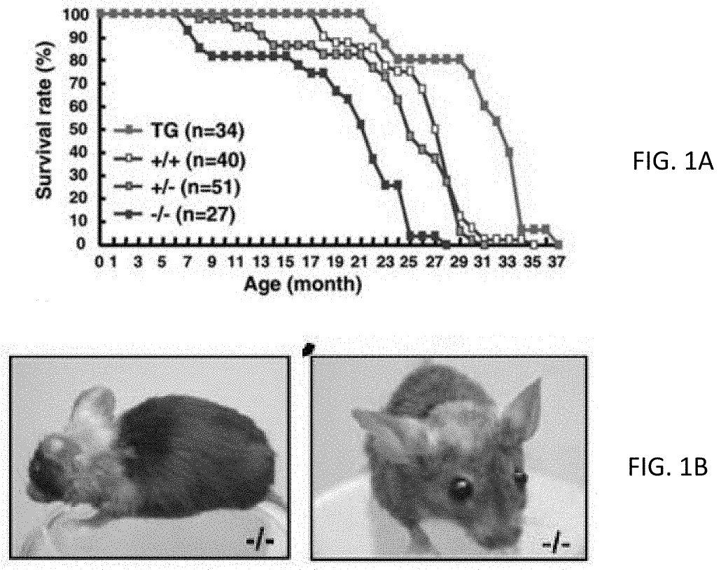

Gene therapies are also the main contestants for treatment of progerias. An example of one such disease is Wolfram Syndrome II—a progeria characterized by diabetes, deafness, cataracts, loss of vision and hearing, atrophy of optic nerves, kidney and GI failure, and a number of other health problems, with average lifespan of about 30 years [5,6]. Wolfram Syndrome II was found to be caused by homozygous loss-of-function mutation in Cisd2—a small protein active in the mitochondrial membrane and endoplasmic reticulum (ER) [7,8]. Cisd2 loss in mice leads to decreased lifespan and phenocopy of most human Wolfram Syndrome II symptoms (FIGS. 1A-1B) [9]. Levels of Cisd2 decrease with age in mice [9], whereas overexpression of Cisd2 increases health and lifespan in mice (FIG. 1A) [10] and possibly humans [7,11]. As such, Cisd2 gene therapy is both a potential treatment of Wolfram Syndrome II and geroprotector to increase healthspan in the general population.

In summary, considering the limitations of small-molecule drugs as geroprotectors, the need for treatment of progerias, and the current major trends of increasing burden of age-related disease, increased knowledge and investment into biology of aging, and increasing efficiency and cost-effectiveness of adeno-associated viruses (AAVs), AAV-based geroprotective gene therapies are on track to become a major part of healthcare.

### DAEUS and the Shortcomings of Current Gene Therapy Methods

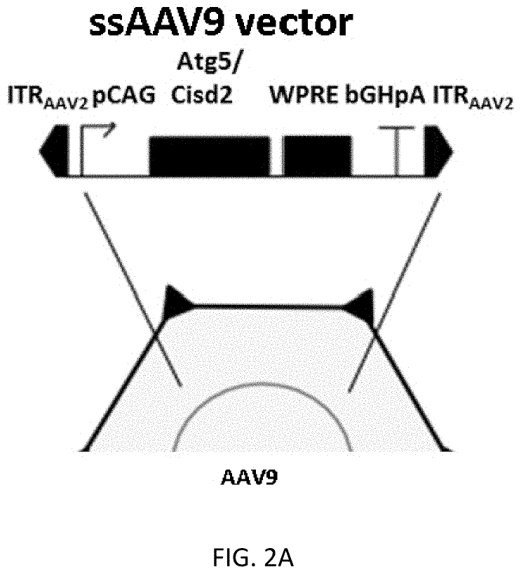

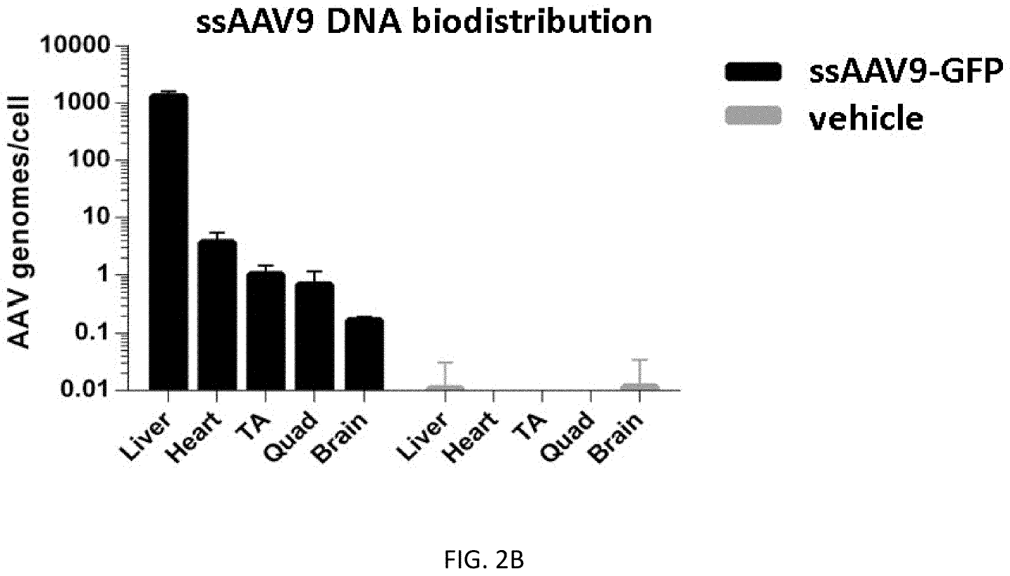

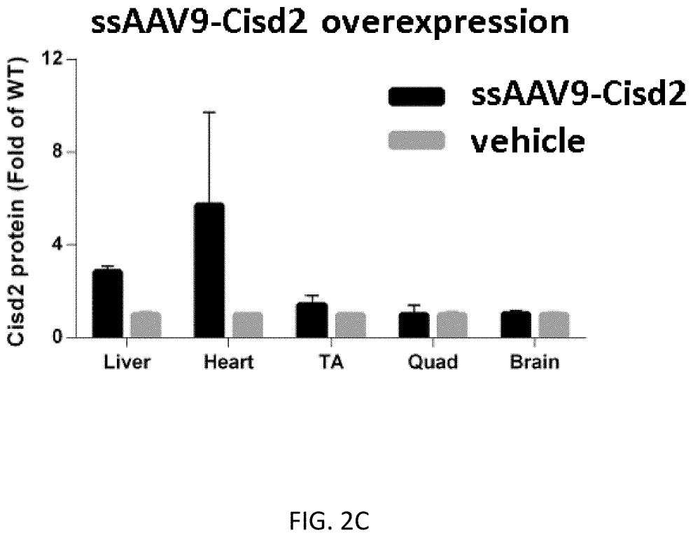

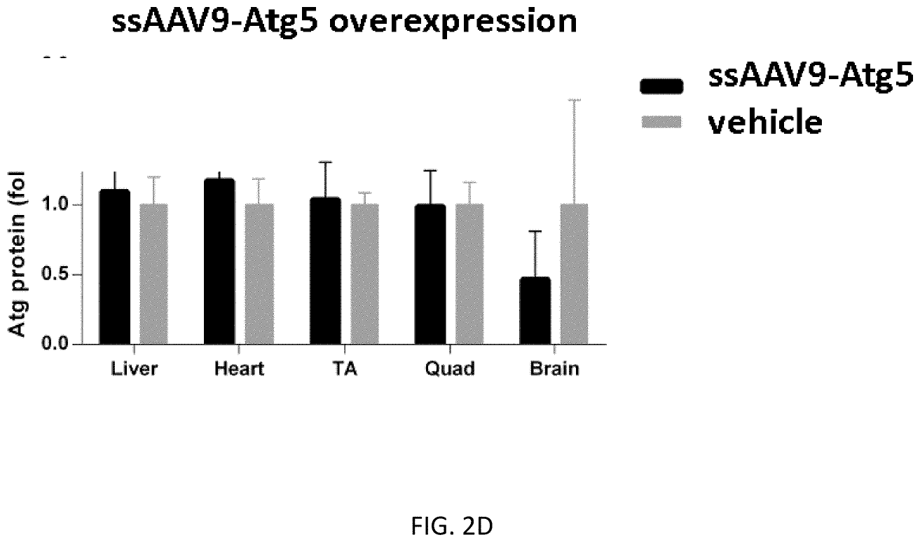

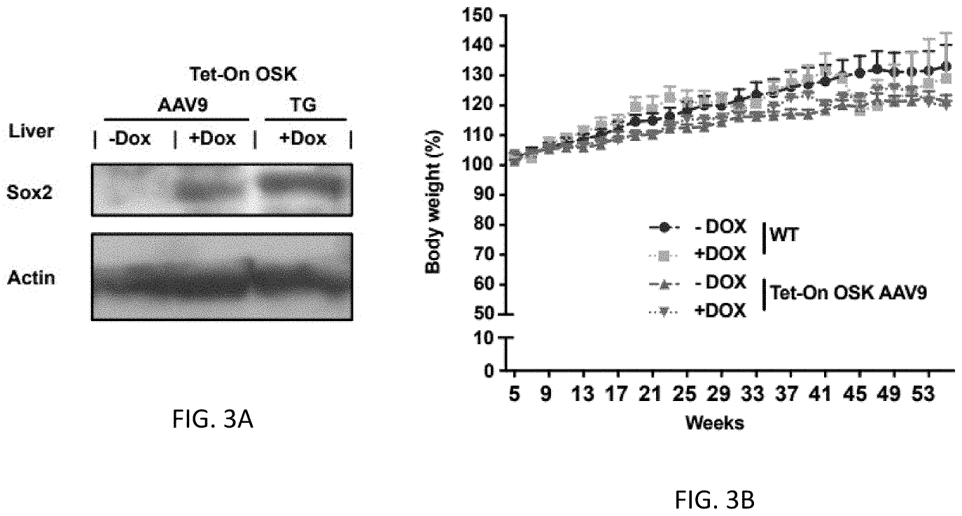

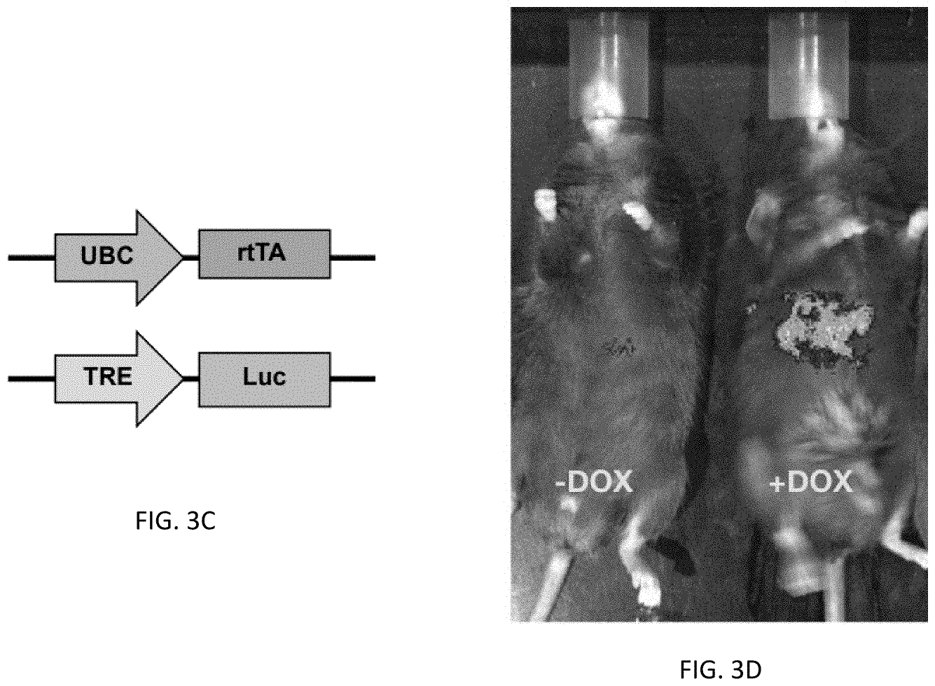

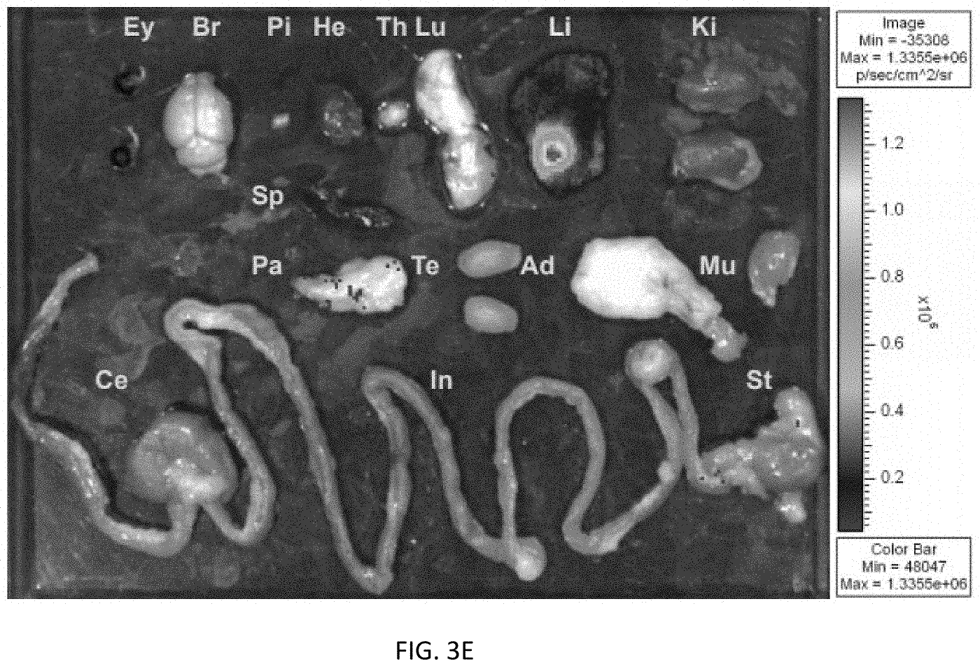

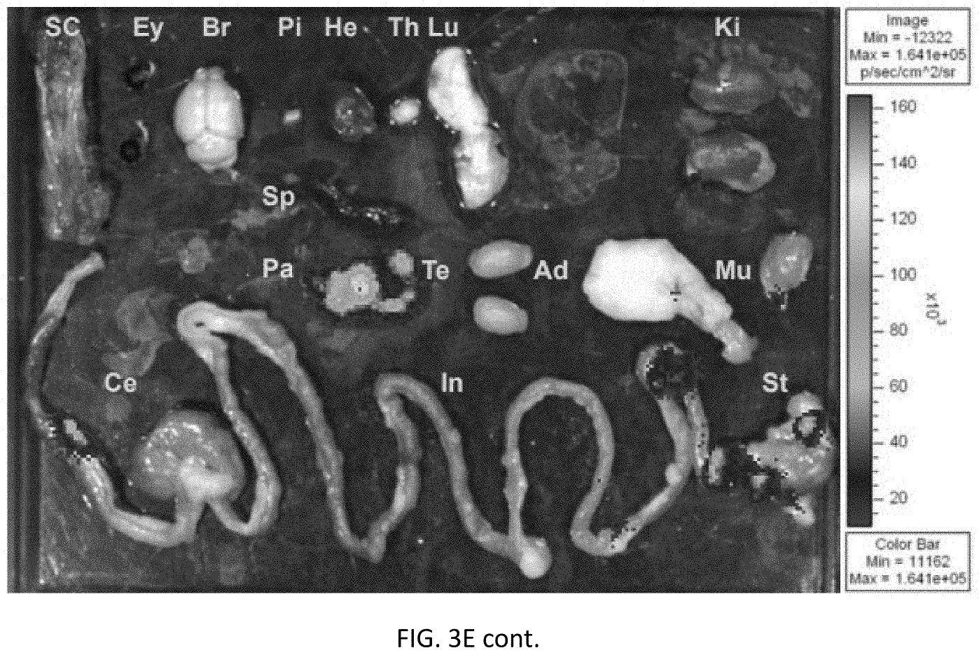

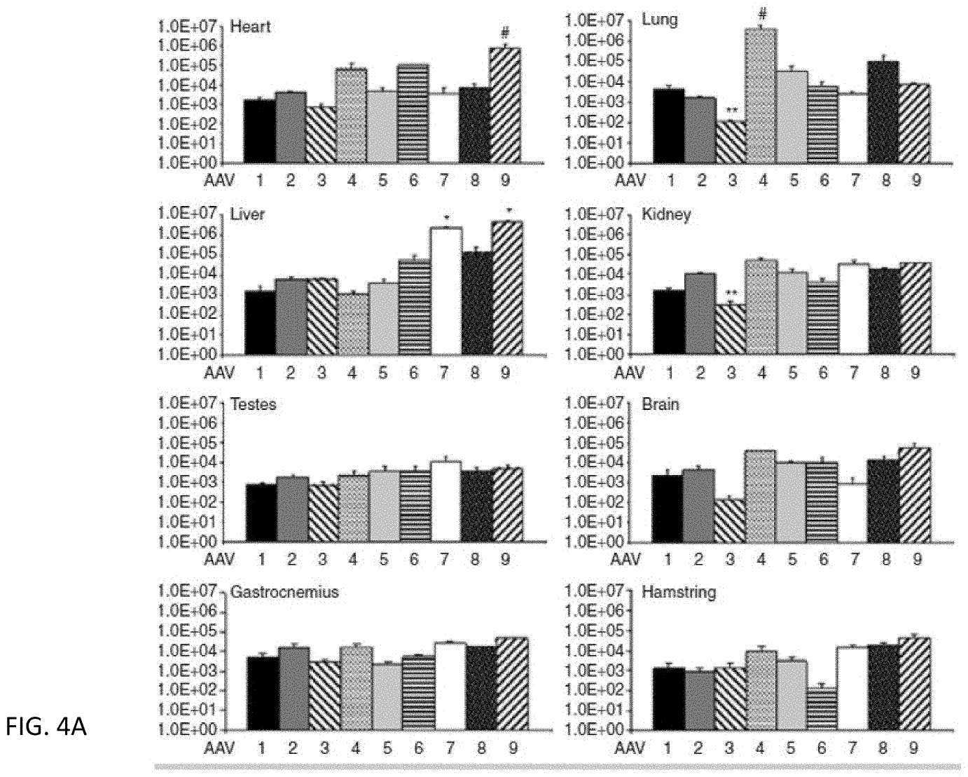

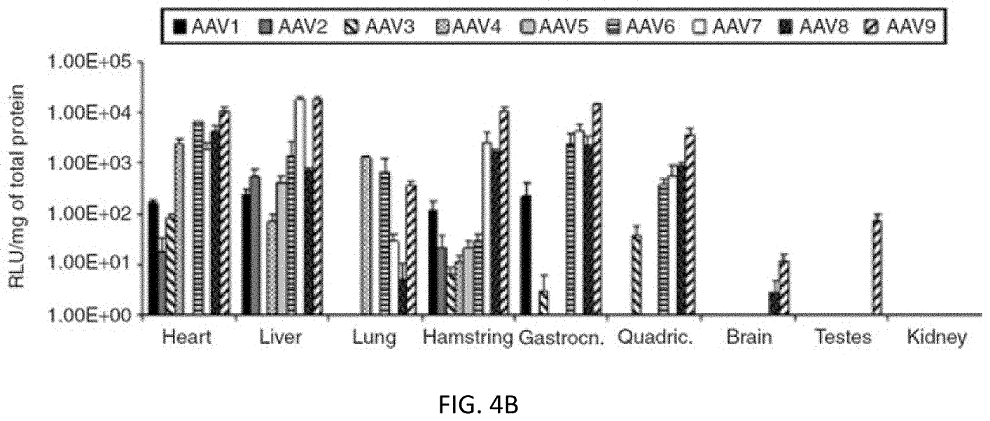

From the various gene therapy methods, adeno-associated viruses (AAVs) are by far the most efficacious and commonly used vectors. From AAVs, one of the most commonly used vectors in both research and new clinical trials are single-stranded AAV9 based vectors (ssAAV9). This is because ssAAV9 can be produced at high titers and can transduce various tissues of the body, with highest expression present in the liver and lowest (by about 100-1000×) in the brain. While there is now a flurry of new engineered and discovered AAV serotypes, ssAAV9 has remained the method of choice as new vectors have either been more difficult to produce (Anc80) or are more efficacious towards a specific tissue only (PHP.B). Similarly to AAV9, other currently existing AAV serotypes result in highly variable gene transfer levels between various tissues. While ssAAV9 is sufficient for some applications, the attempts to use them for aging studies, which require gene delivery to a broad set of tissues, quickly shows that they are not suitable for this purpose. Empirically, it was found that for several geroprotective genes, even optimized ssAAV9 vectors (FIG. 2A) resulted in none or only modest overexpression in aged mice, with high tissue-to-tissue variability (FIGS. 2B-2D). This observation as well as high tissue-to tissue variability has been reported by several others (FIGS. 3-4) [12,13]. This presented a critical roadblock to using AAVs in aging research and therapeutics, and as a result, only three manuscripts using AAVs for aging research appear to have ever been published, all limited to genes that do not require long-term expression or can be secreted or studied in a confined tissue.

To generalize the use of AAVs to effectively deliver most genes involved in aging, a number of technical advances were needed. Firstly, because aging affects the whole body, it is necessary to be able to deliver genes into most tissues of the body, as opposed to a single or a few tissues. Secondly, gene expression must be uniform across these tissues, as opposed to varying multiple orders of magnitude. Third, gene expression must be long-term and stable. Fourth, expression must be strong and efficient to achieve overexpression above wild-type levels (most gene therapies restore expression to only a fraction of wild-type levels). Finally, gene expression must be evenly distributed between individual cells (as opposed to having high cell-to-cell variation as with ssAAV9 vectors).

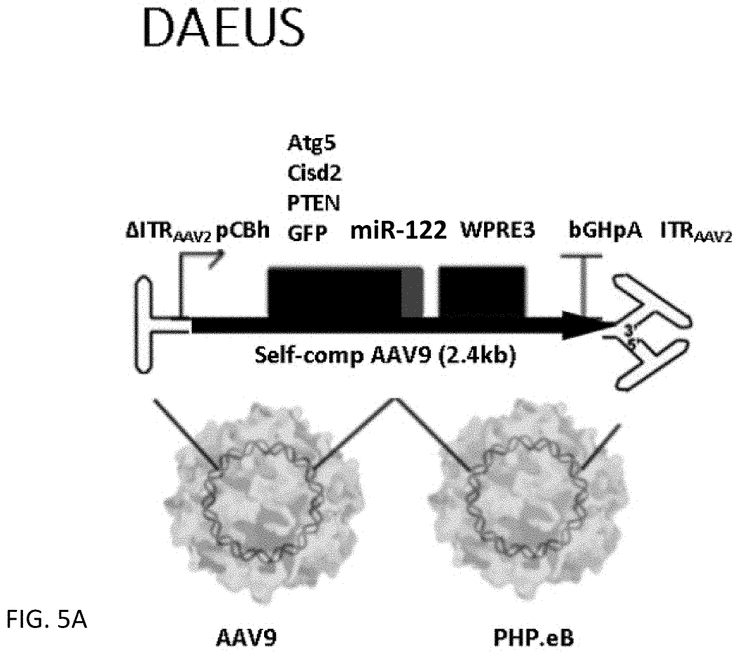

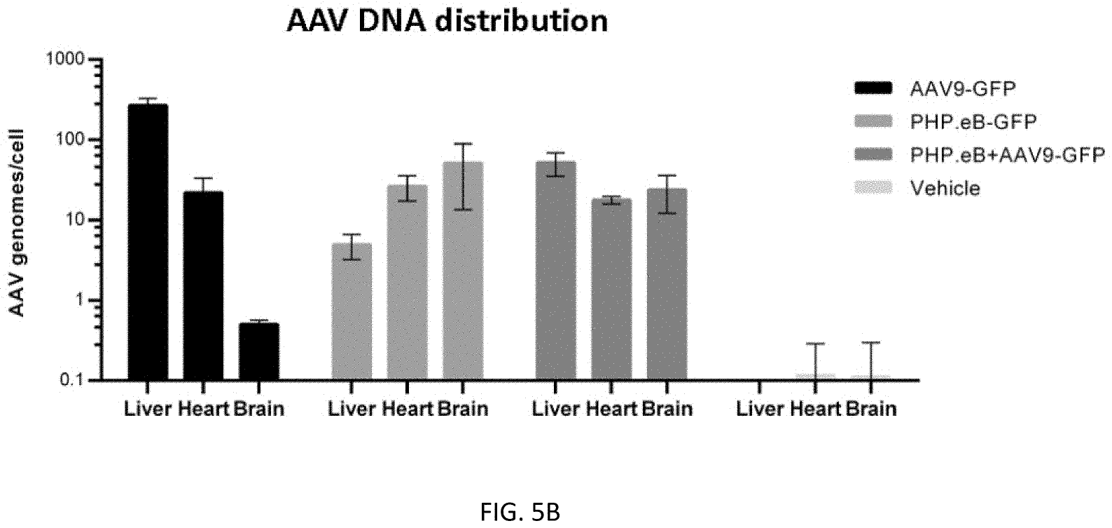

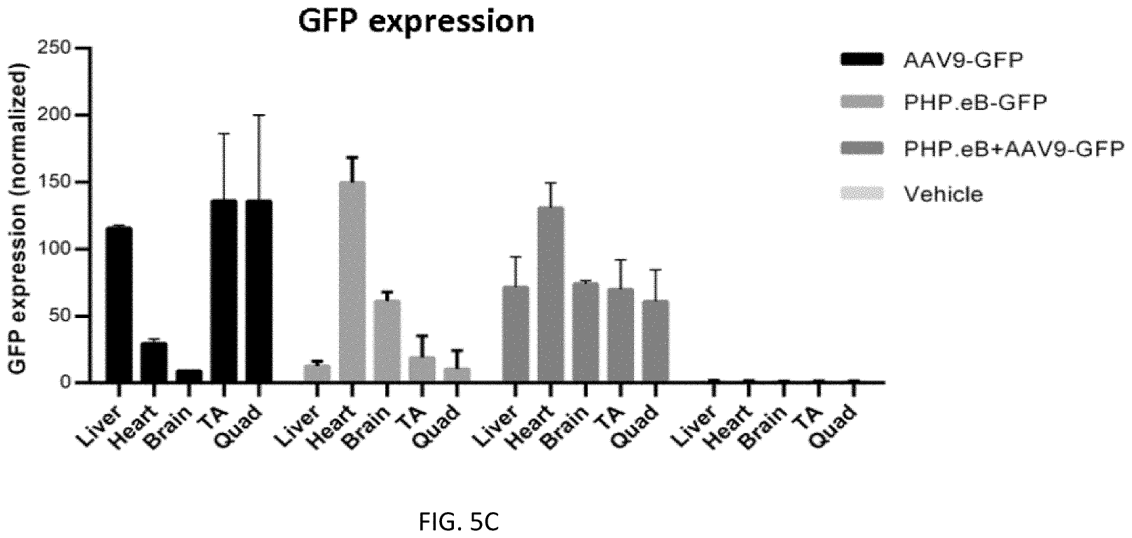

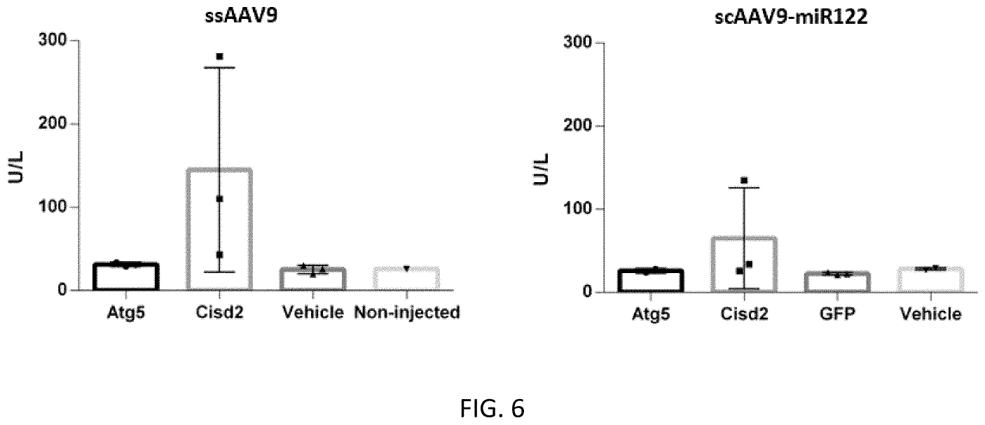

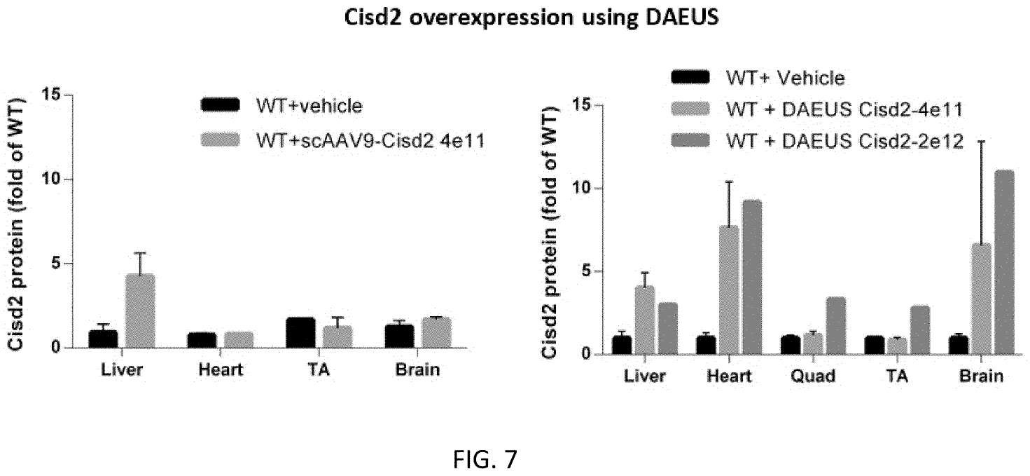

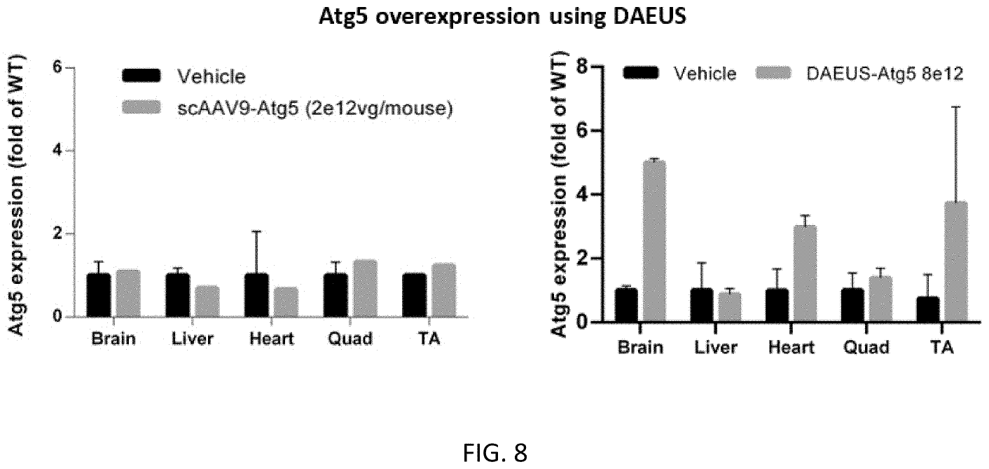

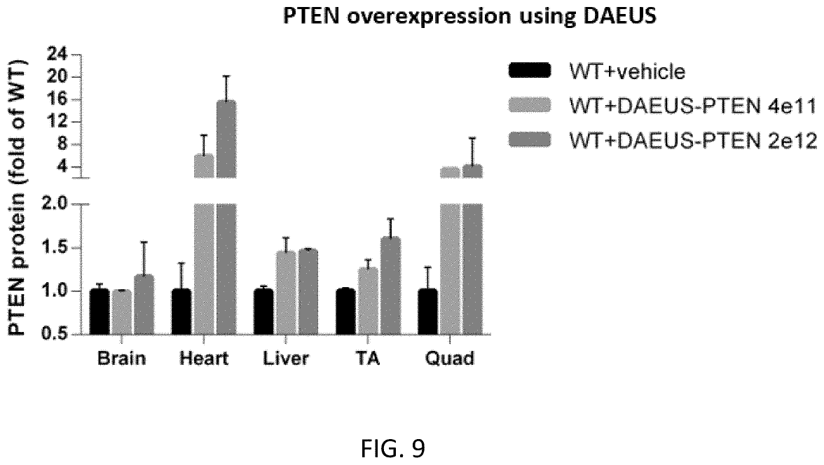

After multiple iterations of testing and development spanning five years and combining resources from two labs, a system that meets these requirements has been developed: DAEUS (Different AAV Expression system for Uniform, Systemic expression). To achieve this, DAEUS employs a newly designed vector architecture using self-complementary vector backbone, two or more AAV serotypes, one or more microRNA target sites, and a strong non-silencing promoter. Specifically, in one example, it uses the chicken (3-actin hybrid (Cbh) promoter to provide expression that is high, long-term and uniform across cells, the liver-specific microRNA 122 target sequence to normalize expression in the liver, codon-optimized gene coding sequences to increase expression further, and two viral serotypes simultaneously (AAV9 and PHP.eB) to deliver genes to most tissues of the body (FIG. 5A). The resulting DAEUS system provided uniform gene transfer and gene expression across major tissues of the body, unlike their components AAV9 and PHP.eB alone (FIGS. 5B-5C). miRNA target sites are included to dampen too high expression in unwanted tissues. In particular, the liver-specific miRNA122 target site was included as the experiments with non-dampened ssAAV9 vectors demonstrated liver toxicity apparent from elevated alanine transaminase (ALT) levels (FIG. 6A). Addition of miR-122 target site decreased toxicity despite the use of more potent vectors (FIG. 6B). Furthermore, at least two serotypes (AAV9 and PHP.eB) were included because the experiments using a single serotype alone, even with an optimized self-complementary backbone containing the Cbh promoter and miR122 target sites showed highly unequal or unsatisfactory expression (FIGS. 5B-5C, FIGS. 7-9). In contrast, using DAEUS, fairly uniform, high level and long-term overexpression of several geroprotective genes in aged wild-type mice was demonstrated (FIGS. 7-9).

Achieving Defined Levels of Gene Transfer and Transgene Expression using DAEUS

To achieve optimal therapeutic efficacy, a defined level of transgene expression across various tissues is often required. The methods described herein employ DAEUS (consisting of multiple different AAV serotypes, such as AAV9, PHP.eB, AAV8, AAV2, etc. in a single cocktail, possibly in conjunction with miRNA target sites on the vector genome, such as miR122 target site, miR182 target site, etc.) to achieve target levels of gene transfer and expression across multiple tissues of the body.

To achieve a defined pattern of gene transfer and gene expression in a subject of a target species, first standard curves of the relationship between injected dose of a specific AAV serotype and the resulting gene transfer level and gene expression at the RNA and/or the protein level are created. To achieve this, individuals of the target species are injected with a specific AAV serotype with doses ranging anywhere between 1e10 to 1e18 AAV vector genomes copies (GC) per kg and the resulting gene transfer and gene expression at the RNA and/or protein levels are measured. This process is repeated singly for every serotype used in the AAV cocktail. This process is also repeated for every miRNA target site used. Additionally, this process is repeated for each pair of AAV serotypes used to estimate possible interaction effects.

Here, gene transfer is defined as AAV vector genome DNA per host cell nuclear genome DNA in a target tissue. RNA expression is defined as transgene RNA counts per million based on next generation sequencing or as transgene RNA levels normalized to host housekeeping gene levels as determined by reverse quantitative PCR or other quantitative RNA assay in a target tissue. Protein expression is defined as levels of transgene protein expression normalized to weight of input tissue, total protein or housekeeping gene protein levels, as assayed by Western Blot, Simple Western, ELISA, or other quantitative protein expression assays in a target tissue.

From these data, standard dose-response curves of AAV dose vs gene transfer and gene expression are estimated using linear or non-linear regression methods for each target tissue. Finally, for each target tissue, the equations derived from regression are summed, including interaction terms, for every AAV serotype and miRNA target site used, providing a model which consists of a set of equations, that allows prediction of the individual doses of AAV serotypes used in the cocktail to achieve target level of gene transfer and gene expression pattern. Any target species, target tissue, AAV serotype and miRNA target site can optionally be used in this method.

A prototype system, based on the methods described above, to achieve target levels of gene transfer in brain, tibialis anterior, heart, liver, and other organs and tissues of house mice (Mus musculus) was engineered. For this end, one embodiment of the DAEUS system employing serotypes AAV9 and PHP.eB and miR122 target site was used.

5-week old male C57BL6-J mice were injected with doses of approximately 5e12, 2e13, 5e13 and 2e14 AAV vector genomes per kg, at N=3 mice per group with the following serotypes and their combinations:
1) scAAV9-Cbh-GFP-miR122,
2) scAAV9-Cbh-GFP-miRScr (where miRNA target site is scrambled to remove its function),
3) scPHP.eB-Cbh-GFP-miR122
4) scPHP.eB-Cbh-GFP-miR122 together with scAAV9-Cbh-GFP-miRScr

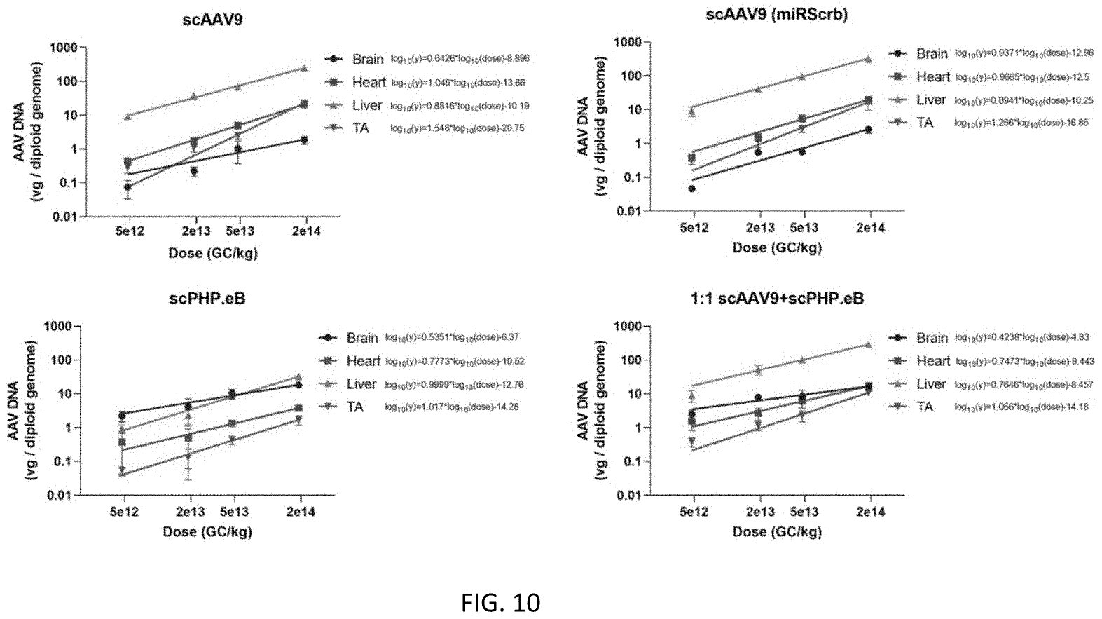

From this, equations of dose-response curves of AAV dose to AAV gene transfer for brain, heart, liver and tibialis anterior were estimated using linear regression (FIG. 10). The specific equations for each serotype and for each tissue are listed in FIG. 10.

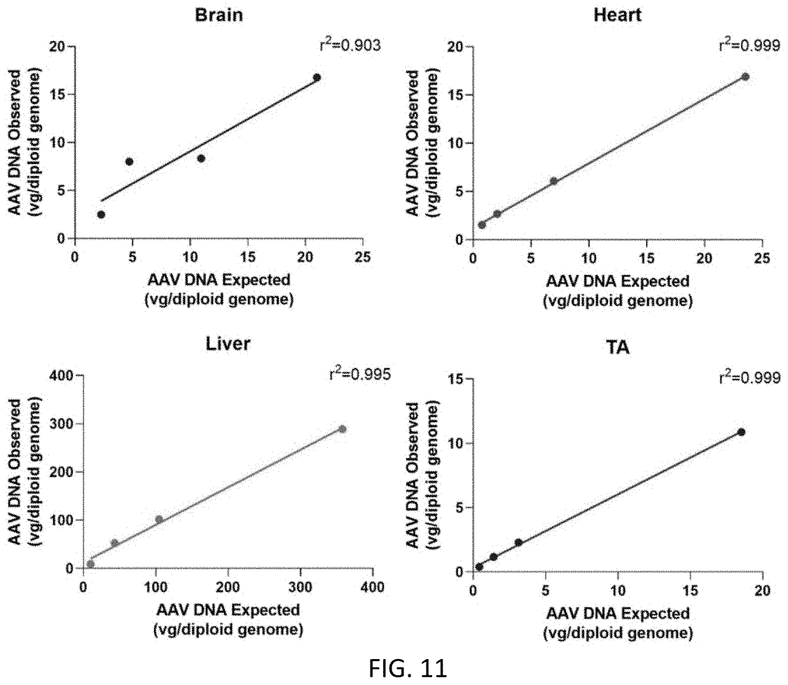

Interaction effects were estimated by summing the gene transfer levels observed in groups 1 and 3 for every tissue individually, and then comparing them to the observed gene transfer levels in group 4. If no interaction is present between AAV9 and PHP.eB, the sum of gene transfer from groups 1 and 3 for every tissue (Expected) should closely match gene transfer levels in group 4 for every tissue respectively (Observed). Regression analysis of expected vs observed gene transfer levels indicated that Expected values matched to and correlated highly with Observed values (r²=0.9 . . . 0.999) (FIG. 11). A two-way ANOVA analysis of interactions indicated no significant interaction between Expected and Observed (FIG. 11). These data indicate that interaction effects between AAV9 and PHP.eB at the doses tested are very minimal or non-existent.

It was then sought to achieve 5 different gene transfer levels of interest. Gene expression patterns were predicted using the model described above of 5 different combinations of AAV9 and PHP.eB doses and 5 groups of 5-week old male C57BL6-J mice were injected retro-orbitally with N=3 mice per group, with the following cocktails:
1) 1.4e14 GC/kg scAAV9-Cbh-GFP-miR122+1.9e13 GC/kg scPHP.eB-Cbh-GFP-miR122
2) 1.9e14 GC/kg scAAV9-Cbh-GFP-miR122+4.8e12 GC/kg scPHP.eB-Cbh-GFP-miR122
3) 4.8e13 GC/kg scAAV9-Cbh-GFP-miR122+1.9e14 GC/kg scPHP.eB-Cbh-GFP-miR122
4) 2.4e13 GC/kg scAAV9-Cbh-GFP-miR122+9.5e12 GC/kg scPHP.eB-Cbh-GFP-miR122
5) 4.8e12 GC/kg scAAV9-Cbh-GFP-miR122+4.8e13 GC/kg scPHP.eB-Cbh-GFP-miR122

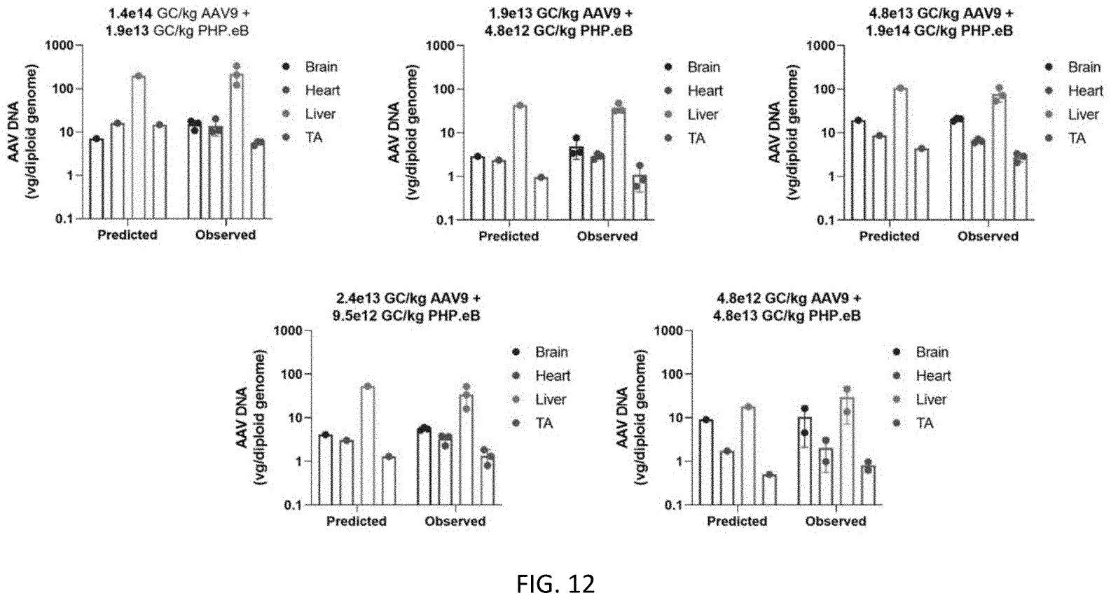

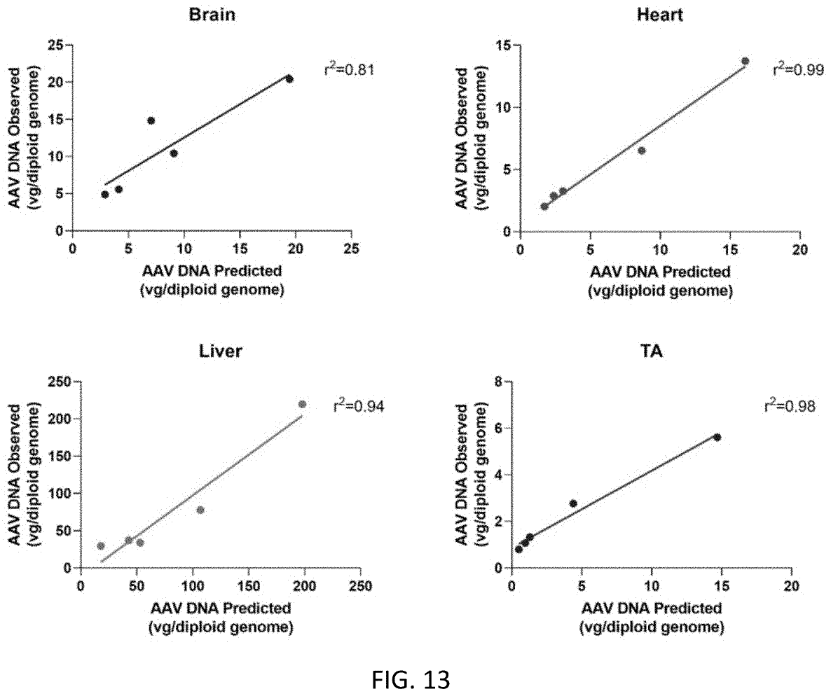

The results indicated a high match between predicted (Predicted) and observed (Observed) gene transfer patterns (FIG. 12). The results also indicated a high correlation of predicted and observed gene transfer levels using linear regression (FIG. 13). This indicates that the DAEUS system, employed in a manner described above, accurately allows pre-determined levels of gene transfer to be achieved.

### Use of DAEUS to Treat Wolfram Syndrome II

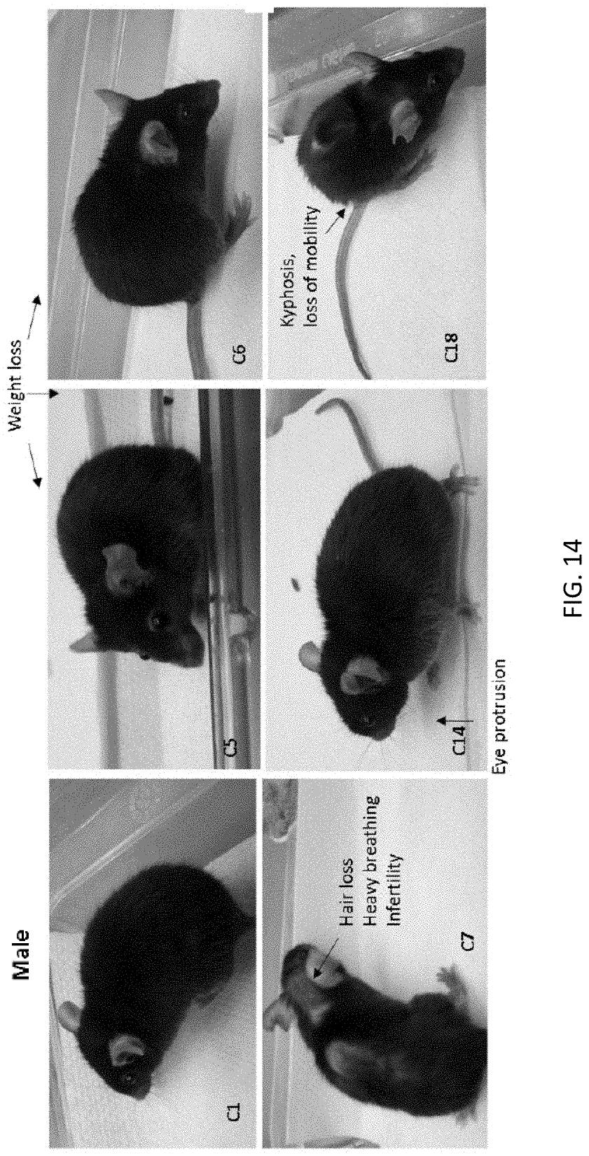

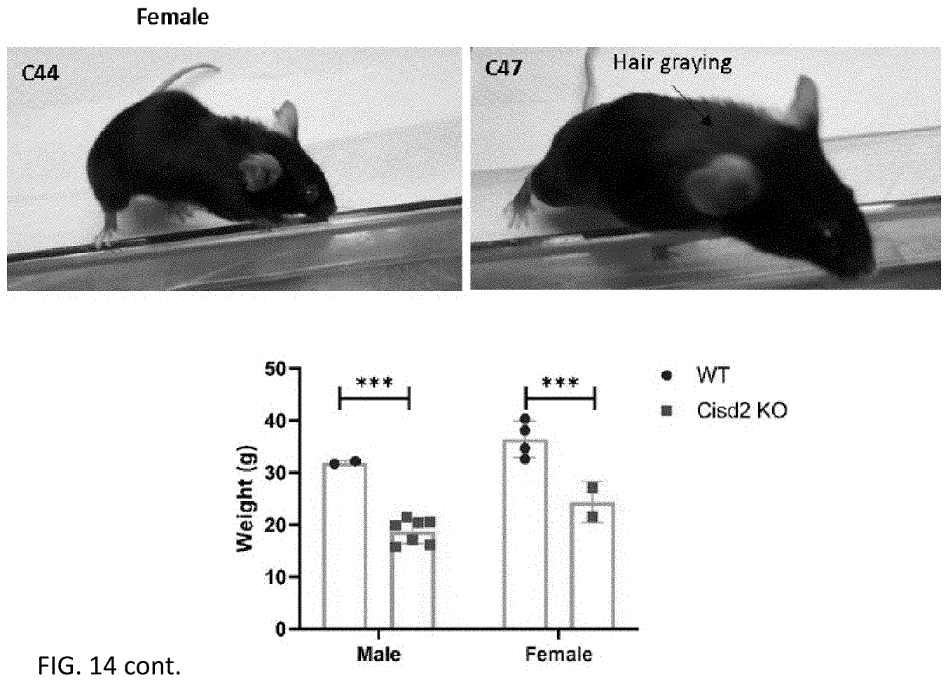

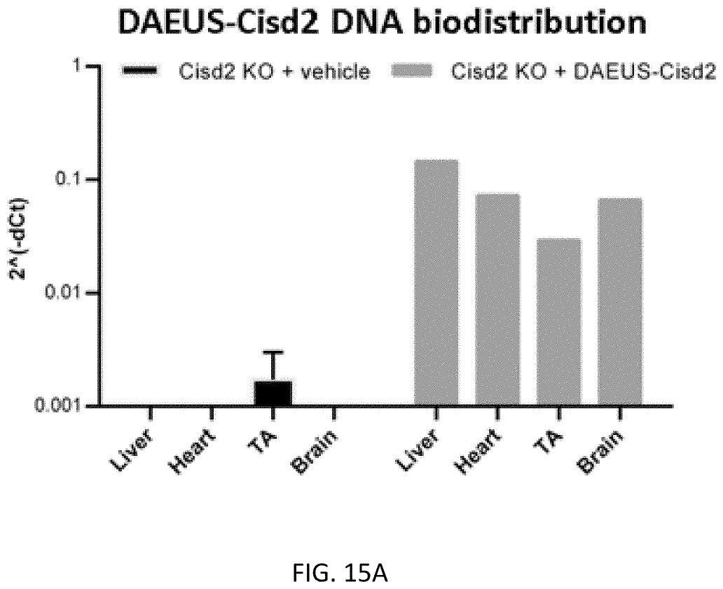

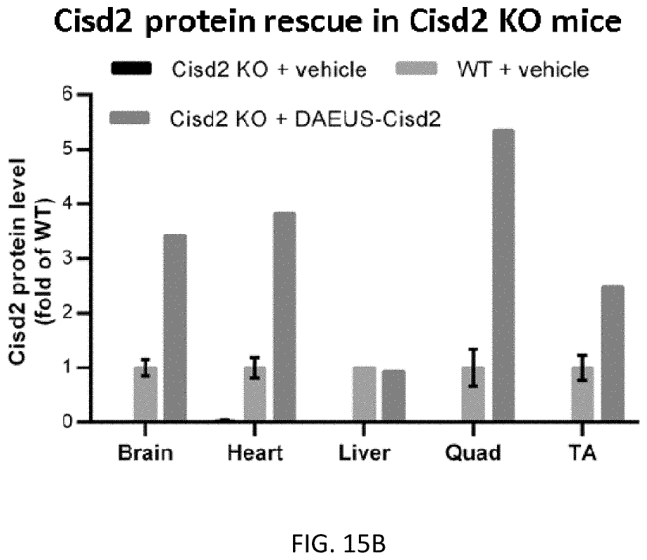

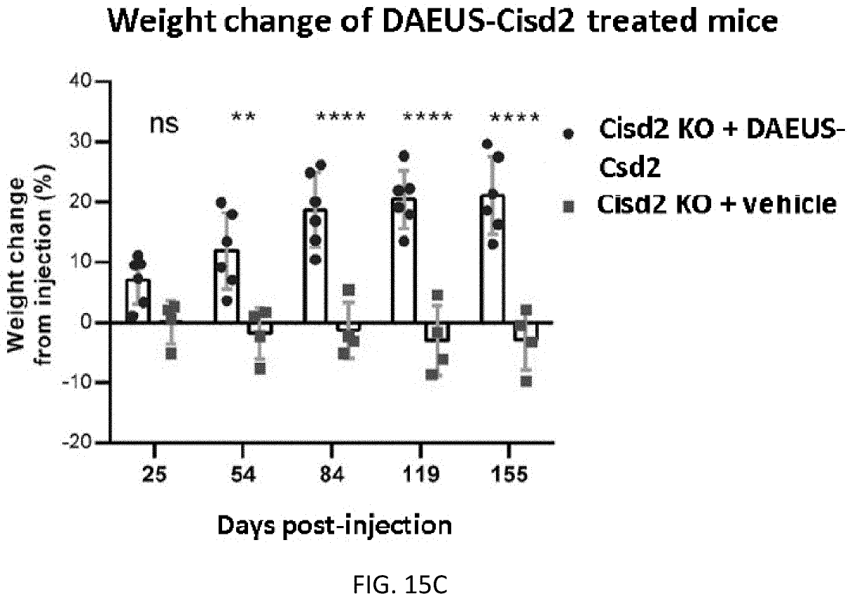

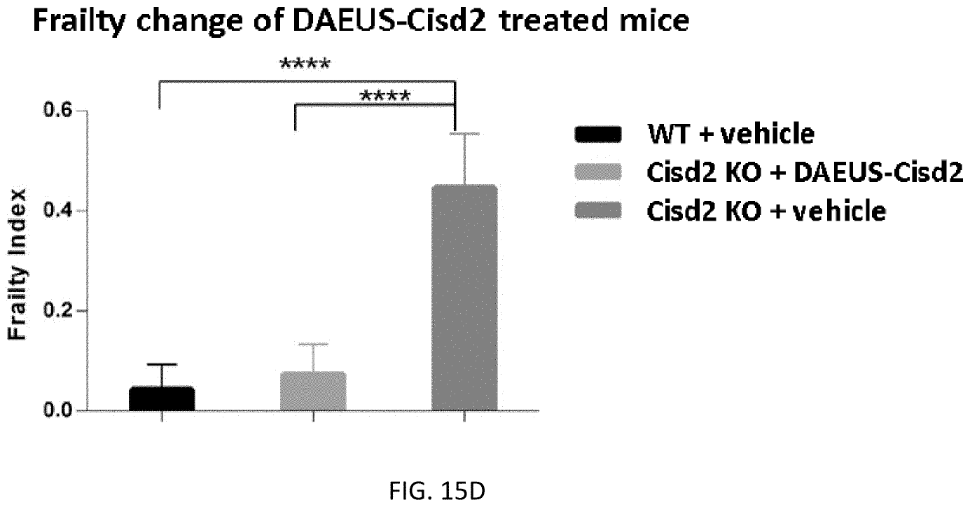

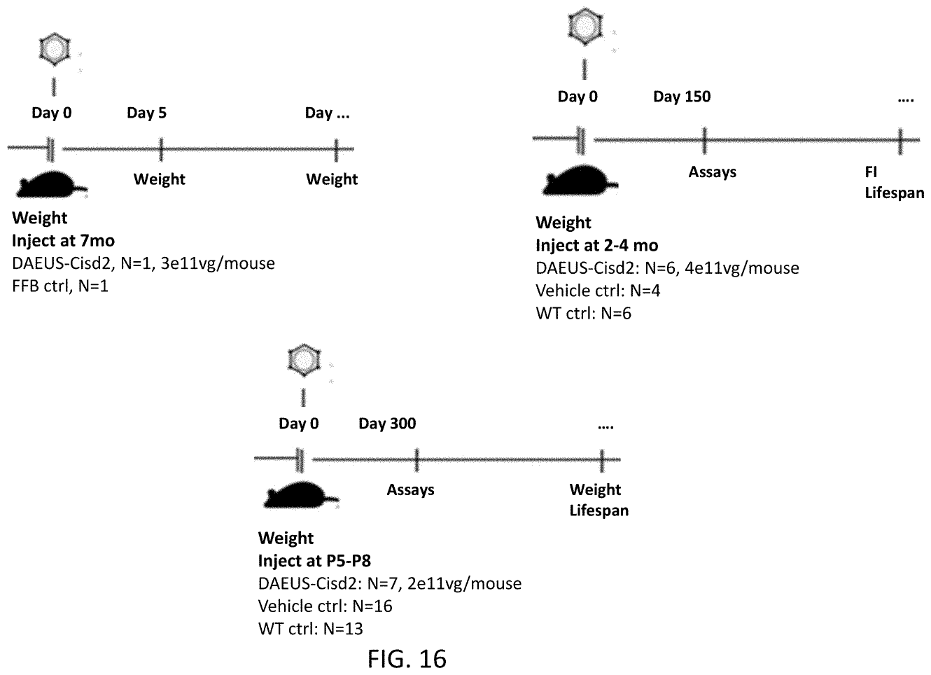

To test the efficacy of DAEUS for treating progerias, lines of Cisd2 knockout mice were established in house (FIG. 14). These are the only non-transgenic Cisd2 knockout models in existence, as they were generated via CRISPR (as opposed to insertional mutagenesis for other models). This model was chosen because as stated above, loss of Cisd2 causes Wolfram Syndrome II, while overexpression of Cisd2 increases healthspan and lifespan in mice and possibly humans [2]. Therefore, Cisd2 gene therapy is potentially both a treatment for Wolfram Syndrome II (WSII) and a geroprotective gene therapy for the general population. With the goal of restoring uniform levels of Cisd2 expression, Cisd2 KO mice were treated with DAEUS-Cisd2 at a total dose of 2e13 vector genomes/kg across various stages of the disease. Treatment of mice with DAEUS-Cisd2 at this dose indeed resulted in uniform restoration of Cisd2 gene transfer (FIG. 15A) and Cisd2 protein expression to physiological levels across multiple tissues (FIG. 15B). This significantly decreased morbidity and mortality across all age groups tested (e.g., mice injected as neonates, at 2-4 months old, or at 7 months old) (FIGS. 15-16).

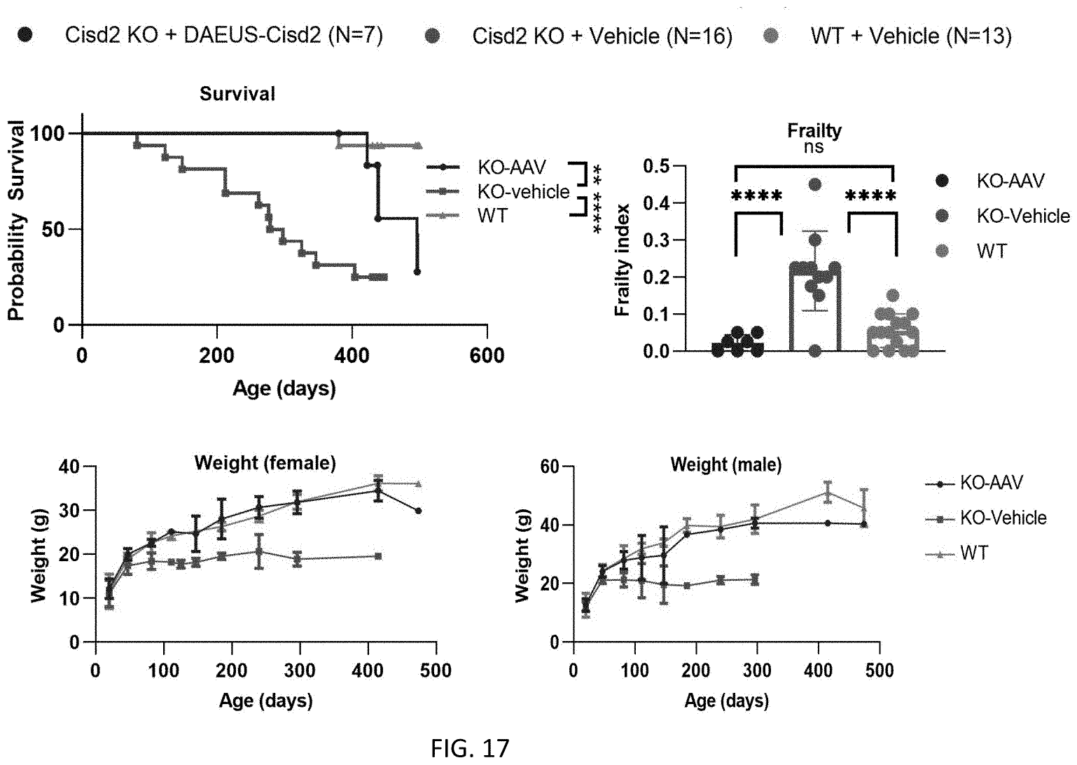

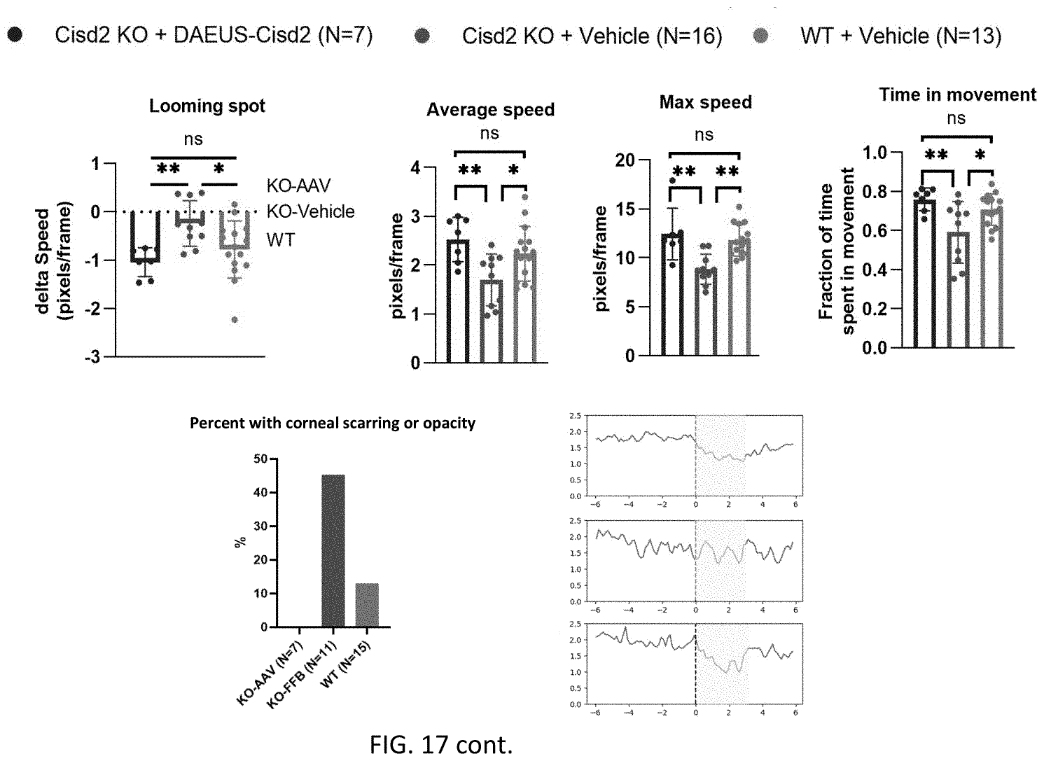

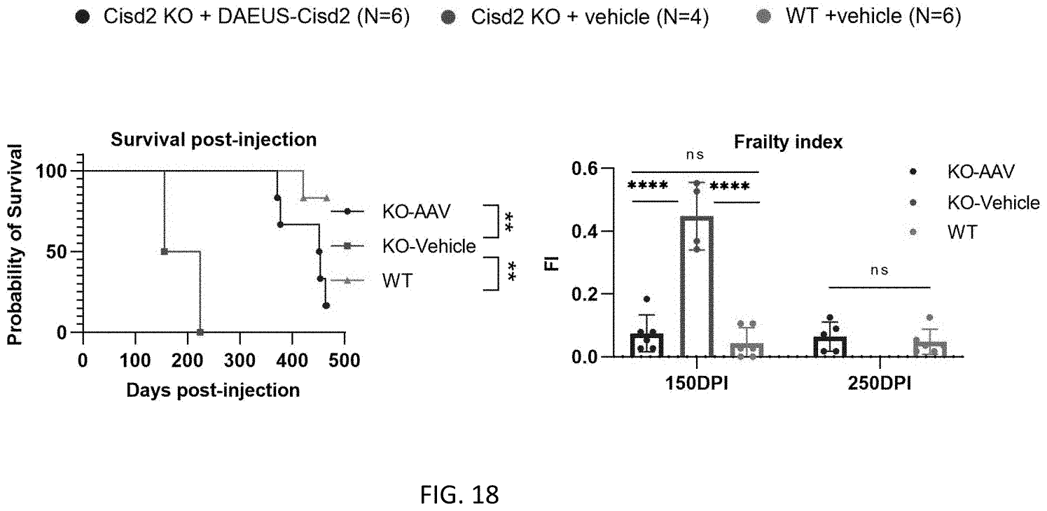

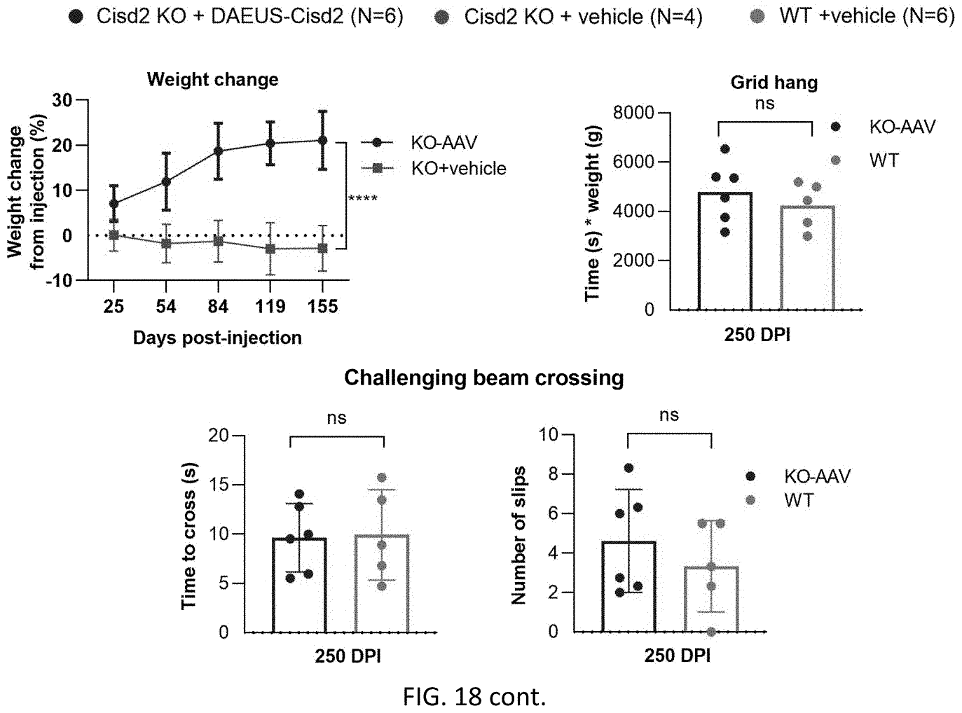

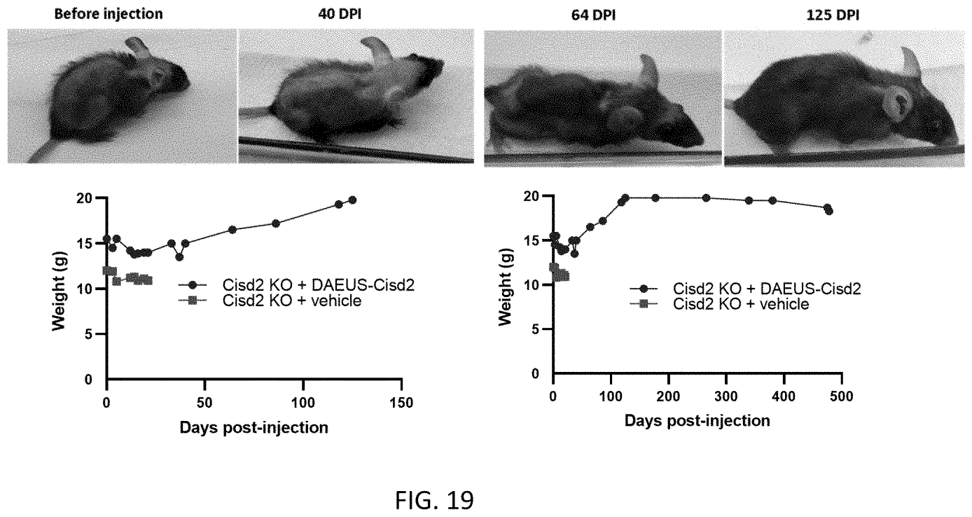

In mice injected as neonates, frailty, weight loss, activity, and vision (assayed as looming spot) were maintained at wild-type levels by DAEUS-Cis2 treatment in comparison to the untreated Cisd2 knockout mice, which saw increased morbidity in all of these functions (FIG. 17). Additionally, lifespan of DAEUS-Cisd2 treated mice was extended approximately two-fold compared to untreated controls (FIG. 17). In mice treated at 2-4 months old, frailty, weight loss, muscle strength (assayed as grid hand), and coordination (assayed as challenging beam crossing) were improved compared to untreated controls (FIG. 18). In addition, lifespan increased by about two-fold (FIG. 18). Strikingly, in mice with advanced disease, DAEUS-Cisd2 treatment reversed weight loss, hair loss, kyphosis and other morbidities and extends lifespan by three-fold (FIG. 19). This data demonstrates that DAEUS-Cisd2 is a highly effective therapy for the prevention and treatment of Wolfram Syndrome II.

Use of DAEUS to extend lifespan of wild-type mice

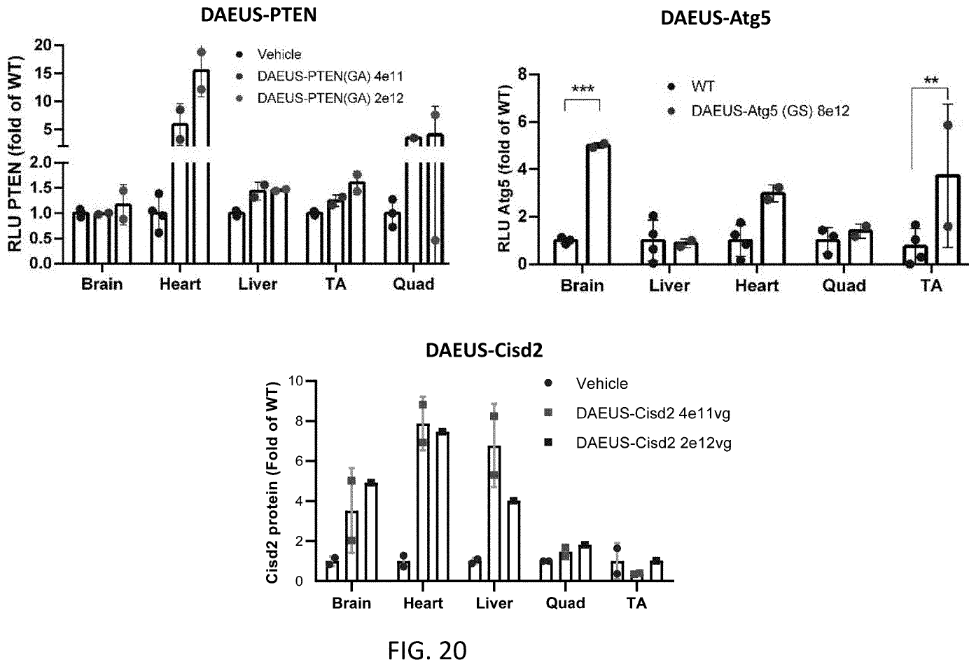

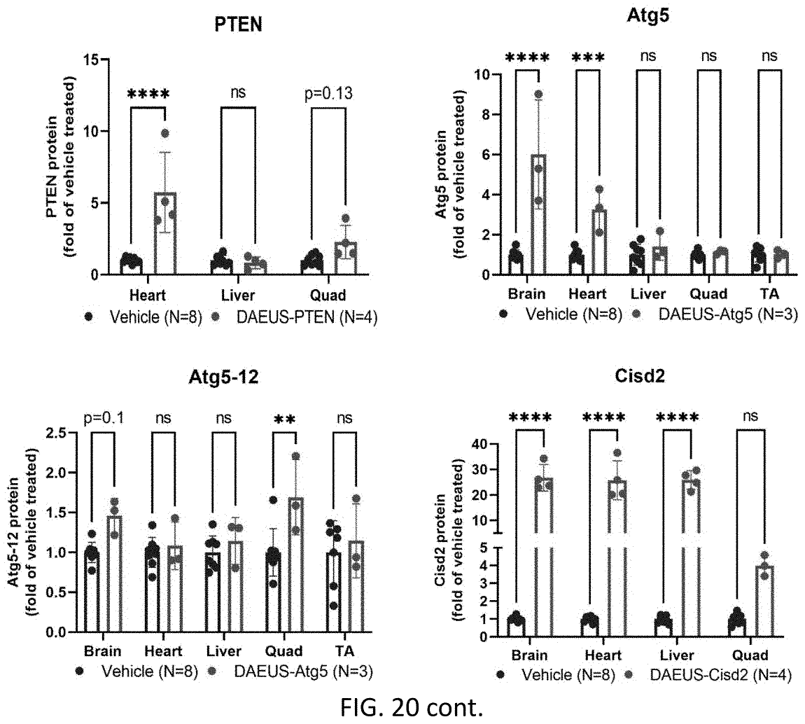

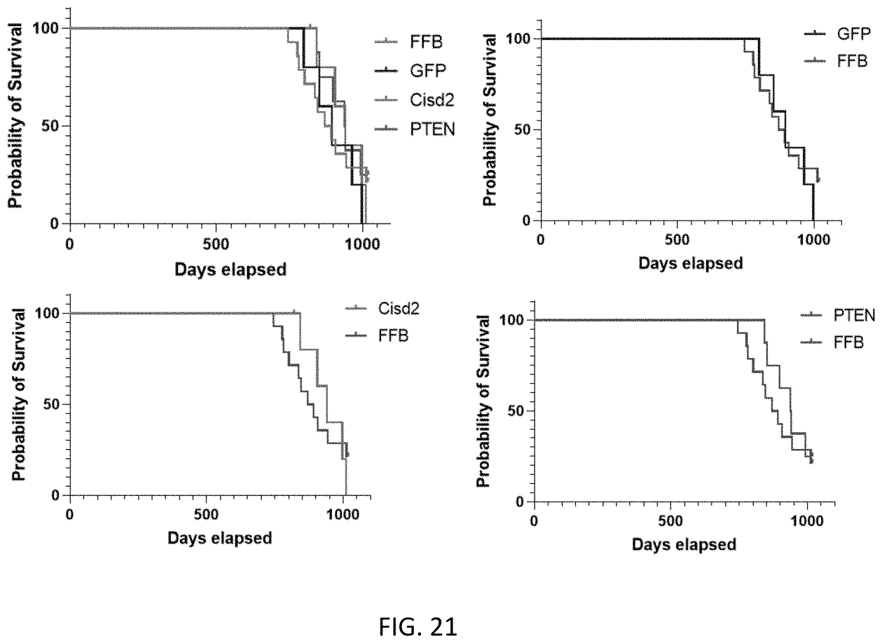

Next, a DAEUS system was engineered to overexpress geroprotective genes Cisd2, Atg5, and PTEN in wild-type (not progeroid) mice with the goal of extending the lifespan of treated mice. For this purpose, the ability to overexpress Cisd2, Atg5, and PTEN above wild-type levels in wild-type mice was verified by delivering DAEUS-Atg5, DAEUS-PTEN, and DAEUS-Cisd2 at optimized doses into 18 month old wild-type mice, and measuring the resulting protein expression 1 month post-injection. In two sets of experiments, overexpression of all three genes using optimized doses of DAEUS across multiple major tissues of the body were demonstrated (FIG. 20). Then the effect of DAEUS-Cisd2 and DAEUS-PTEN treatment on the lifespan of wild-type C57BL6/J mice was tested. For this end, equal numbers of male and female 24 month old mice were injected retro-orbitally with 2e12 vg/mouse of DAEUS-Cisd2, 1e12 vg/mouse of DAEUS-PTEN, or 1e12 vg/mouse of DAEUS-GFP or vehicle (FFB) as controls. The lifespans of the treated groups were then measured and the results indicated that DAEUS-Cisd2 and DAEUS-PTEN treated mice did show longer lifespans compared to DAEUS-GFP or vehicle treated mice (DAEUS-Cisd2: 7% increase in overall median lifespan and 38% increase in post-injection lifespan; DAEUS-PTEN: 7% increase in overall median lifespan and 37% increase in post-injection lifespan) (FIG. 21). The results demonstrate that the DAEUS system described herein can be used to overexpress the geroprotective genes and extend the lifespan of treated subjects.

### Materials and Methods

### Construction of Plasmids and Plasmid Sequences

ssAAV9 and DAEUS vectors were constructed by DNA synthesis and cloning. The ITR to ITR sequence of DAEUS vectors were fully synthesized and cloned into pAAV\SC\CMV\EGFP\WPRE\bGH-2 backbone (received from Vandenberghe lab) using standard molecular cloning. ssAAV9 vectors were partially synthesized and cloned into the AAV pCAG-FLEX2-tTA2-WPRE-bGHpA backbone (Addgene). For ssAAV9 vectors, native Mus musculus coding sequences were used. For DAEUS vectors, Atg5 and PTEN coding sequences were codon optimized.

### AAV Production and Purification

HEK293 cells at 80% confluency from four 15cm dishes were seeded to a hyperflask, grown to 80% confluency and triple-transfected with AAV vector, Rep/Cap for AAV8 or AAV9 (Addgene 112864 and 112865) and pAdΔF6 at 130 ug:130 ug:260 ug per hyperflask respectively. Four days post-transfection, supernatant from a hyperflask was decanted into a 1 L flask and 3 ml Triton-X 100 (8787-100 ML Millipore Sigma), 2.5 mg RNAse A at 1 mg/ml concentration (10109142001 Millipore Sigma), 25 U/mL of Turbonuclease (ACGC80007 VitaScientific) and 56 μl of 10% Pluronic F68 (24040032 Thermo Fisher) was added to the supernatant. The supernatant was then mixed, poured back into the hyperflask, and shaken on an orbital shaker at 150 rpm at 37° C. for 1 hour to lyse the cells and remove plasmid DNA. Lysate was then decanted from the hyperflask, and the hyperflask washed with 140 mL of DPBS (10010072 Life Tech) which was added to the rest of the lysate. The total lysate was then centrifuged at 4000 g, 4° C. for 30 min, and the supernatant was filtered through a 0.45 μm PES bottle-top filter (295-4545 Thermo Fisher) before loading onto HPLC.

AAV purification was performed using AAVX POROS CaptureSelect (ThermoFisher Scientific) resin with 6.6mm×100mm column (Glass, Omnifit, kinesis-USA) in an Akta Pure HPLC system containing an auxiliary sample pump (GE LifeSciences). The machine was setup at room temperature and all purifications were performed at room temperature (approximately 21° C.). Column volume [CV] for each purification was 1 mL. The chromatography column was pre-equilibrated with 10 [CV] of wash buffer 1X Tris-buffered Saline (1×TBS) (Boston Bioproducts), before application of the AAV lysate. Equilibration and all subsequent washes of the column were performed at a rate of 2 ml/minute.

The clarified/filtered lysate containing the AAV virions was loaded at a rate of 1 mL/minute onto AAVX POROS column, with total loading time ranging from 30 minutes for small-scale preparations to 700 minutes (overnight) for hyperflasks. In later purifications a loading rate of 1.5 mL/min was also used to decrease total run time and no decrease in purification efficiency was observed. The column containing bound AAV was then washed with 10 [CV] of 1×TBS, followed by washes of 5 [CV] of 2×TBS, 10 [CV] 20% EtOH and 10 [CV] 1×TBS wash. The bound AAV was eluted using a low-pH (pH 2.5 . . . 2.9) buffer of 0.2M Glycine in 1×TBS at a rate of 1 ml/minute. Elution fractions were taken as 0.25-1 mL volumes per fraction. The eluted virus solution was neutralized by adding 1M Tris-HCL (pH 8.0) at 1/10th of the fraction volume directly into the fraction collection tube prior to elution. Peak fractions based on UV (280 nm) absorption graphs were collected and buffer exchanged in final formulation buffer (FFB: 1×PBS, 172 mM NaCl, 0.001% pluronic F68) and concentrated using an Amicon filter with a molecular weight cut-off of 50 kDa (UFC905008 EMD Millipore) prior to virus titration.

In brief, viral titer and the genomic titer was determined by a quantitative PCR (TaqMan, Life Technologies). Real-time qPCR (7500 Real-Time PCR System; Applied Biosystems, Foster City, Calif., USA) with BghpA-targeted primer-probes (GCCAGCCATCTGTTGT (SEQ ID NO: 1), GGAGTGGCACCTTCCA (SEQ ID NO: 2), 6FAM-TCCCCCGTGCCTTCCTTGACC-TAMRA (SEQ ID NO: 3)) was used. Linearized CBA-EGFP DNA was used at a series of dilutions of known concentration as a standard. After 95° C. holding stage for 10 seconds, two-step PCR cycling stage was performed at 95° C. for 5 seconds, followed by 60° C. for 5 seconds for 40 cycles. Genomic vector titers were interpolated from the standard and expressed as vector genomes per milliliter.

### DNA and Protein Quantification

Tissues were homogenized by disrupting 30mg of tissue in 1 mL of RLT+ buffer for DNA and RNA and 1 mL of RIPA buffer containing 1× Halt protease and phosphatase inhibitors for protein (78444 Thermo Fisher Sci). For disruption, samples, buffer and 1 mm Zirconia/Silica beads (11079110z Biospec) were loaded into XXTuff vials (330TX BioSpec) and disrupted using Mini Beadbeater 24 (112011 BioSpec) at max speed for 3 minutes. Vials were then placed on ice for 2-5 minutes for RNA and 1 hour for protein, centrifuged at 10,000 g for 3 min and supernatant used for further procedures.

For DNA/RNA, 700 μL of supernatant was loaded onto AllPrep DNA Mini Spin Columns and purified using AllPrep DNA/RNA/miRNA Universal Kit (80224 Qiagen) for quadriceps and Allprep DNA/RNA mini kit (80204 Qiagen) for brain and liver. Purification was performed on Qiacube Connect (9002864 Qiagen).

Total AAV copy number was assessed using BghpA primers and linearized CBA-GFP plasmid dilution series as standard for AAV copy number (GCCAGCCATCTGTTGT (SEQ ID NO: 1), GGAGTGGCACCTTCCA (SEQ ID NO: 2), 6FAM-TCCCCCGTGCCTTCCTTGACC-TAMRA (SEQ ID NO: 3)). Total genome copy number was estimated using RPII primers-probes (GTTTTCATCACTGTTCATGATGC (SEQ ID NO: 4), TCATGGGCATTACTATTCCTAC (SEQ ID NO: 5), probe: VIC-AGGACCAGCTTCTCTGCATTATCATCGTTGAAGAT-3IABkFQ (SEQ ID NO: 6)) along with a standard of gDNA dilution series of known concentration. AAV copy number per diploid genome was then calculated as

$$copy number per diploid genome = 2 * ( \frac{total A A V copy number}{total genome copy number} ) .$$

Efficiency and specificity of amplification for both primer-probe sets was previously established, and amplification was performed using Luna Universal Probe qPCR

Master Mix (M3004 L NEB) at thermocycling conditions recommended by the manufacturer.

For quantification of protein expression, protein lysate was first diluted 5× twice in fresh RIPA+Halt inhibitors buffer and all dilutions were assayed for total protein content using PierceTM BCA Protein Assay Kit (23225 Thermo Fisher). For each tissue type, lysates were then diluted in RIPA+Halt inhibitors buffer to the concentration where they would be at the lower end of the linear range. For GFP, anti-GFP antibody ab290 (ab290 Abcam) was used. For Cisd2, PTEN and Atg5, anti-Cisd2 (13318-1-AP Proteintech), anti-Atg5 (NB110-53818 Novus) and anti-PTEN D4.3 (Cell Signaling) antibodies, respectively, were used. Linear range for protein quantification was previously determined by assaying each protein separately using 12-230 kDa Jess or Wes Separation Module (SM-W004 Protein Simple) on Wes with ab290 for dilutions ranging from 3 μg/μl . . . 0.03 μg/μl for each tissue. Linear range for total protein was also previously determined by assaying total protein in the range of 4 μg/μl . . . 0.1 μg/μl using Total Protein Detection Module (DM-TP01 Protein Simple) (linear range: <1 μg/μl for all tissues tested). GFP, Atg5, Cisd2 and PTEN as well as total protein levels were then assayed and GFP and total protein quantified using Compass for SW 4.1 (Protein Simple). Finally, GFP was normalized to total protein to arrive at the final value.

Animal experiments

Mice were housed in standard ventilated racks at a maximum density of 5 mice per cage. Room temperature was maintained at 22° C. with 30%-70% humidity. Mice were kept on a 12-hour light/dark cycle and provided food and water ad libitum. Breeder mice were kept on irradiated PicoLab Mouse Diet 20 5058 (LabDiet, St. Louis, Mo.), and non-breeder mice were kept on irradiated LabDiet Prolab Isopro RMH 3000 5P75 (LabDiet, St. Louis, Mo.). Cages were filled with ¼ inch Anderson's Bed o Cob bedding (The Andersons, Inc., Maumee, Ohio.) and every cage contained three nestlet (2 3 2″ compressed cotton square, Ancare, Bellmore, N.Y.) and one red mouse hut (certified polycarbonate; 3 ¾″ wide x 1 ⅞″ tall×3″ long, BioServ, Flemington, N.J.). Cage changes were performed at least every 14 days, and more frequently if necessary. Animal health surveillance was performed quarterly by PCR testing of index animals and through swabs from rack plenums AAV was injected retro-orbitally under isofluorane anesthesia in a volume of 150 μl per mouse at various total doses as described in text and in figures. AAV9 and PHP.eB were used in 1:1 ratios for injections of DAEUS-Atg5, DAEUS-Cisd2, DAEUS-GFP and DAEUS-PTEN, 8-week old or 18-month old wild-type C57BL/6J mice were used as described in text and in figures. Mice were CO2 euthanized 28 days post-injection and tissues and serum collected for analysis, except as otherwise noted in the text and in figures. Serum ALT levels were quantified by UMass Mouse Metabolic Phenotyping Center.

### Generation of Cisd2 KO Mice and Frailty Measurements

Cisd2 knockout mice were generated via microinjection of C57BL6/J fertilized oocytes with SpCas9 protein and three guide RNAs targeting Exon 2 of Cisd2 (AGCGCAAGTACCCCGAGGAA (SEQ ID NO: 7), CCCCGAGGAAGGGCAGTAGG (SEQ ID NO: 8), TGCTGTGTTCAGTTTCAGAC (SEQ ID NO: 9)). Founders were then genotyped and Sanger sequenced (primers AGCCCTAAGTTTCTCCGAGTTC (SEQ ID NO: 10), GTGACATGTGGTGCTGTAGAAC (SEQ ID NO: 11)), and founders with loss-of-function mutation bred to WT C57BL6/J. Pups were then backcrossed further to WT C57BL6/J mice. Heterozygous pups of this backcross were then bred to arrive at homozygous Cisd2 knockout mice. Two lines were bred further (Line 6: deletion of 780bp, deletion of whole exon 2 and Line 14: deletion of 261 bp, frameshift due to deletion of most of exon 2, 4 bp left at 3′ of exon 2). Loss of Cisd2 expression was confirmed via Simple Wes (not shown). Mice were then weighed at intervals and frailty assessed 4 months post-injection. Frailty was assessed blinded as the weighted sum of 31 morbidity related measures as described in Whitehead et al. [14], with the exception that non-informative measures (measures that were 0 or 1 across all mice) were excluded from final analysis.

### Statistical Analysis

All data was visualized and statistical analysis was performed in GraphPad Prism (GraphPad). Specific statistical tests used are listed in figure legends for each test, and all tests were performed with default settings unless otherwise specified.

Exemplary viral vectors
LOCUS scAAV-CbhM-Atg5(GS)-miR 5237 bp ds-DNA circular DEFINITION.

```
FEATURES  Location/Qualifiers
  CDS  3937..4797
    /label=“Amp-R”
    /ApEinfo_revcolor=#f58a5e
    /ApEinfo_fwdcolor=#f58a5e
  misc_feature 1089..1109
    /label=“CAG3 F”
    /ApEinfo_revcolor=#faac61
    /ApEinfo_fwdcolor=#faac61
  misc_feature 2568..2575
    /label=“Seed region”
    /ApEinfo_revcolor=#faac61
    /ApEinfo_fwdcolor=#faac61
  misc_feature 2583..2829
    /label=“WPRE3 correct”
    /ApEinfo_revcolor=#f8d3a9
    /ApEinfo_fwdcolor=#f8d3a9
  misc_feature 1609..1700
    /label=“Cbh 3′ cloning site”
    /ApEinfo_revcolor=#c6c9d1
    /ApEinfo_fwdcolor=#c6c9d1
  misc_feature 1701..1701
    /label=“cloning scar”
    /ApEinfo_revcolor=#ff9ccd
    /ApEinfo_fwdcolor=#ff9ccd
  repeat_region 767..872
    /label=“5′-ITR”
    /ApEinfo_revcolor=#c6c9d1
    /ApEinfo_fwdcolor=#c6c9d1
  misc_feature 1682..1700
    /label=“3′ end of hybrid intron”
    /ApEinfo_revcolor=#b7e6d7
    /ApEinfo_fwdcolor=#b7e6d7
  misc_feature 2547..2576
    /label=“mIR122 target site m8”
    /ApEinfo_revcolor=#9eafd2
    /ApEinfo_fwdcolor=#9eafd2
  misc_feature 1701..1706
    /label=“cloning scar”
    /ApEinfo_revcolor=#85dae9
    /ApEinfo_fwdcolor=#85dae9
  misc_feature 889..1700
    /label=“Cbh”
    /ApEinfo_revcolor=#d59687
    /ApEinfo_fwdcolor=#d59687
  misc_feature 1180..1201
    /label=“CAG3 R”
    /ApEinfo_revcolor=#c6c9d1
    /ApEinfo_fwdcolor=#c6c9d1
  misc_feature 2304..2321
    /label=“Shared region to WT”
    /ApEinfo_revcolor=#b7e6d7
    /ApEinfo_fwdcolor=#b7e6d7
  misc_feature 2442..2467
    /label=“shared region to WT”
    /ApEinfo_revcolor=#c7b0e3
    /ApEinfo_fwdcolor=#c7b0e3
  misc_feature 1472..1700
    /label=“Hybrid intron”
    /ApEinfo_revcolor=#b7e6d7
    /ApEinfo_fwdcolor=#b7e6d7
  misc_feature 3045..3068
    /label=“deleted in 5′ ITR”
    /ApEinfo_revcolor=#c7b0e3
    /ApEinfo_fwdcolor=#c7b0e3
  polyA_signal 2830..3044
    /label=“BGH\pA”
    /ApEinfo_revcolor=#faac61
    /ApEinfo_fwdcolor=#faac61
  misc_feature 1713..2540
    /label=“Atg5 CO Genscript”
    /ApEinfo_revcolor=#b7e6d7
    /ApEinfo_fwdcolor=#b7e6d7
  misc_feature 2717..2741
    /label=“oMF80”
    /ApEinfo_revcolor=#faac61
    /ApEinfo_fwdcolor=#faac61
  enhancer 889..1192
    /label=“CMV enhancer”
    /ApEinfo_revcolor=#f58a5e
    /ApEinfo_fwdcolor=#f58a5e
  misc_feature 883..887
    /label=“Cbh 5′ cloning site”
    /ApEinfo_revcolor=#b4abac
    /ApEinfo_fwdcolor=#b4abac
  misc_feature 1707..1712
    /label=“Kozak”
    /ApEinfo_revcolor=#ff9ccd
    /ApEinfo_fwdcolor=#ff9ccd
  repeat_region complement(3045..3174)
    /label=“3′-ITR”
    /ApEinfo_revcolor=#ffef86
    /ApEinfo_fwdcolor=#ffef86
  promoter 1194..1471
    /label=“chicken beta-actin promoter”
    /ApEinfo_revcolor=#c7b0e3
    /ApEinfo_fwdcolor=#c7b0e3
```

ORIGIN

```
(SEQ ID NO: 12)
      1 gttggactca agacgatagt taccggataa ggcgcagcgg tcgggctgaa cggggggttc
    61 gtgcacacag cccagcttgg agcgaacgac ctacaccgaa ctgagatacc tacagcgtga
  121 gctatgagaa agcgccacgc ttcccgaagg gagaaaggcg gacaggtatc cggtaagcgg
  181 cagggtcgga acaggagagc gcacgaggga gcttccaggg ggaaacgcct ggtatcttta
  241 tagtcctgtc gggtttcgcc acctctgact tgagcgtcga tttttgtgat gctcgtcagg
  301 ggggcggagc ctatggaaaa acgccagcaa cgcggccttt ttacggttcc tggccttttg
  361 ctgGCCtttt gctcacatgt tctttcctgc gttatcccct gattctgtgg ataaccgtat
  421 taccgccttt gagtgagctg ataccgctcg ccgcagccga acgaccgagc gcagcgagtc
  481 agtgagcgag gaagcggaag agcgcccaat acgcaaaccg cctctccccg cgcgttggcc
  541 gattcattaa tgcagctggc acgacaggtt tcccgactgg aaagcgggca gtgagcgcaa
  601 cgcaattaat gtgagttagc tcactcatta ggcaccccag gctttacact ttatgcttcc
  661 ggctcgtatg ttgtgtggaa ttgtgagcgg ataacaattt cacacaggaa acagctatga
  721 ccatgattac gccagattta attaagggat ctgggccact ccctctctgc gcgctcgctc
  781 gctcactgag gccgggcgac caaaggtcgc ccgacgcccg ggctttgccc gggcggcctc
  841 agtgagcgag cgagcgcgca gagagggagt ggttaagCTA GCggtacccg ttacataact
  901 tacggtaaat ggcccgcctg gctgaccgcc caacgacccc cgcccattga cgtcaataat
  961 gacgtatgtt cccatagtaa cgccaatagg gactttccat tgacgtcaat gggtggagta
1021 tttacggtaa actgcccact tggcagtaca tcaagtgtat catatgccaa gtacgccccc
1081 tattgacgtc aatgacggta aatggcccgc ctggcattat gcccagtaca tgaccttatg
1141 ggactttcct acttggcagt acatctacgt attagtcatc gctattacca tggtcgaggt
1201 gagccccacg ttctgcttca ctctccccat ctcccccccc tccccacccc caattttgta
1261 tttatttatt ttttaattat tttgtgcagc gatgggggcg gggggggggg gggggcgcgc
1321 gccaggcggg gcggggcggg gcgaggggcg gggcggggcg aggcggagag gtgcggcggc
1381 agccaatcag agcggcgcgc tccgaaagtt tccttttatg gcgaggcggc ggcggcggcg
1441 gccctataaa aagcgaagcg cgcggcgggc gggagtcgct gcgacgctgc cttcgccccg
1501 tgccccgctc cgccgccgcc tcgcgccgcc cgccccggct ctgactgacc gcgttactcc
1561 cacaggtgag cgggcgggac ggcccttctc ctccgggctg taattagctg agcaagaggt
1621 aagggtttaa gggatggttg gttggtgggg tattaatgtt taattacctg gagcacctgc
1681 ctgaaatcac tttttttcag acgcgtgcca ccatgacgga tgataaagat gttctgagag
1741 atgtctggtt cggacgcatt cctacctgct tcacgctgta ccaagatgag attacggaga
1801 gggaggctga accctactac ctgctgctgc caagagtcag ctacctgact ctggtgaccg
1861 acaaggtcaa gaagcacttc cagaaggtca tgaggcagga ggacgtgtct gaaatctggt
1921 tcgagtacga aggaactcct ctgaagtggc actaccccat cggtctgctg ttcgacctgc
1981 tggcttccag ctctgccctg ccttggaaca tcaccgtcca cttcaagagc ttcccagaga
2041 aggacctgct gcactgccct tcaaaggacg ctgtggaggc ccacttcatg tcctgcatga
2101 aggaagctga cgccctgaag cacaagtccc aggtcatcaa cgaaatgcag aagaaggacc
2161 acaagcagct gtggatgggt ctgcaaaacg accgcttcga ccagttctgg gctatcaacc
2221 gtaagctgat ggagtaccct cctgaggaaa acggcttccg ctacatcccc ttccgtatct
2281 accagaccac taccgaaagg cccttcatcc agaagctgtt cagaccagtg gctgccgacg
2341 gtcagctgca cactctgggc gacctgctga gggaggtctg cccatcagct gtggctcctg
2401 aggacggaga aaagaggagc caggtcatga tccacggaat cgagccaatg ctggaaaccc
2461 ctctgcaatg gctgtccgaa cacctctcct acccagacaa cttcctccac atttccattg
2521 tcccccaacc tacggactaa aagcttatcg cgaacaaaca ccattgtcac actccaacta
2581 gtataatcaa cctctggatt acaaaatttg tgaaagattg actggtattc ttaactatgt
2641 tgctcctttt acgctatgtg gatacgctgc tttaatgcct ttgtatcatg ctattgcttc
2701 ccgtatggct ttcattttct cctccttgta taaatcctgg ttagttcttg ccacggcgga
2761 actcatcgcc gcctgccttg cccgctgctg gacaggggct cggctgttgg gcactgacaa
2821 ttccgtggtg cctcgactgt gccttctagt tgccagccat ctgttgtttg cccctccccc
2881 gtgccttcct tgaccctgga aggtgccact cccactgtcc tttcctaata aaatgaggaa
2941 attgcatcgc attgtctgag taggtgtcat tctattctgg ggggtggggt ggggcaggac
3001 agcaaggggg aggattggga agacaatagc aggcatgctg gggaaggaac ccctagtgat
3061 ggagttggcc actccctctc tgcgcgctcg ctcgctcact gaggccgggc gaccaaaggt
3121 cgcccgacgc ccgggctttg cccgggcggc ctcagtgagc gagcgagcgc gcagccttaa
3181 ttaacctaat tcactggccg tcgttttaca acgtcgtgac tgggaaaacc ctggcgttac
3241 ccaacttaat cgccttgcag cacatccccc tttcgccagc tggcgtaata gcgaagaggc
3301 ccgcaccgat cgcccttccc aacagttgcg cagcctgaat ggcgaatggg acgcgccctg
3361 tagcggcgca ttaagcgcgg cgggtgtggt ggttacgcgc agcgtgaccg ctacacttgc
3421 cagcgcccta gcgcccgctc ctttcgcttt cttcccttcc tttctcgcca cgttcgccgg
3481 ctttccccgt caagctctaa atcgggggct ccctttaggg ttccgattta gtgctttacg
3541 gcacctcgac cccaaaaaac ttgattaggg tgatggttca cgtagtgggc catcgccctg
3601 atagacggtt tttcgccctt tgacgttgga gtccacgttc tttaatagtg gactcttgtt
3661 ccaaactgga acaacactca accctatctc ggtctattct tttgatttat aagggatttt
3721 gccgatttcg gcctattggt taaaaaatga gctgatttaa caaaaattta acgcgaattt
3781 taacaaaata ttaacgctta caatttaggt ggcacttttc ggggaaatgt gcgcggaacc
3841 cctatttgtt tatttttcta aatacattca aatatgtatc cgctcatgag acaataaccc
3901 tgataaatgc ttcaataata ttgaaaaagg aagagtatga gtattcaaca tttccgtgtc
3961 gcccttattc ccttttttgc ggcattttgc cttcctgttt ttgctcaccc agaaacgctg
4021 gtgaaagtaa aagatgctga agatcagttg ggtgcacgag tgggttacat cgaactggat
4081 ctcaaCAGCg gtaagatcct tgagagtttt cgccccgaag aacgttttcc aatgatgagc
4141 acttttaaag ttctgctatg tggcgcggta ttatcccgta ttgacgccgg gcaagagcaa
4201 ctcggtcgcc gcatacacta ttctcagaat gacttggttg agtactcacc agtcacagaa
4261 aagcatctta cggatggcat gacagtaaga gaattatgca gtgctgccat aaccatgagt
4321 gataacactg cggccaactt acttctgaca acgatcggag gaccgaagga gctaaccgct
4381 tttttgcaca acatggggga tcatgtaact cgccttgatc gttgggaacc ggagctgaat
4441 gaagccatac caaacgacga gcgtgacacc acgatgcctg tagcaatggc aacaacgttg
4501 cgcaaactat taactggcga actacttact ctagcttccc ggcaacaatt aatagactgg
4561 atggaggcgg ataaagttgc aggaccactt ctgcgctcgg cccttccggc tggctggttt
4621 attgctgata aatctggagc cggtgagcgt gggtctcgcg gtatcattgc agcactgggg
4681 ccagatggta agccctcccg tatcgtagtt atctacacga cggggagtca ggcaactatg
4741 gatgaacgaa atagacagat cgctgagata ggtgcctcac tgattaagca ttggtaactg
4801 tcagaccaag tttactcata tatactttag attgatttaa aacttcattt ttaatttaaa
4861 aggatctagg tgaagatcct ttttgataat ctcatgacca aaatccctta acgtgagttt
4921 tcgttccact gagcgtcaga ccccgtagaa aagatcaaag gatcttcttg agatcctttt
4981 tttctgcgcg taatctgctg cttgcaaaca aaaaaaccac cgctaccagc ggtggtttgt
5041 ttgccggatc aagagctacc aactcttttt ccgaaggtaa ctggcttcag cagagcgcag
5101 ataccaaata ctgtTcttct agtgtagccg tagttaggcc accacttcaa gaactctgta
5161 gcaccgccta catacctcgc tctgctaatc ctgttaccag tggctgctgc cagtggcgat
5221 aagtcgtgtc ttaccgg
```

LOCUS scAAV-CbhM-Cisd2-miR122 4817 bp ds-DNA circular DEFINITION.

| | |
|---|---|
| FEATURES | Location/Qualifiers |
| STS | 1294..1354 |
| | /label=“Cisd2 STS” |
| | /ApEinfo_revcolor=#f58a5e |
| | /ApEinfo_fwdcolor=#f58a5e |
| misc_feature | 941..946 |
| | /label=“Kozak” |
| | /ApEinfo_revcolor=#ff9ccd |
| | /ApEinfo_fwdcolor=#ff9ccd |
| polyA_signal | 1644..1858 |
| | /label=“BGH\pA” |
| | /ApEinfo_revcolor=#faac61 |
| | /ApEinfo_fwdcolor=#faac61 |
| misc_feature | 1261..1283 |
| | /label=“oMF155 Reverse” |
| | /ApEinfo_revcolor=#d59687 |
| | /ApEinfo_fwdcolor=#d59687 |
| CDS | 2751..3611 |
| | /label=“Amp-R” |
| | /ApEinfo_revcolor=#f58a5e |
| | /ApEinfo_fwdcolor=#f58a5e |
| misc_feature | 800..932 |
| | /label=“stem-loop” |
| | /ApEinfo_revcolor=#84b0dc |
| | /ApEinfo_fwdcolor=#84b0dc |
| misc_feature | 652..760 |
| | /label=“stem-loop” |
| | /ApEinfo_revcolor=#b7e6d7 |
| | /ApEinfo_fwdcolor=#b7e6d7 |
| misc_feature | 1666..1681 |
| | /label=“bGhpA” |
| | /ApEinfo_revcolor=#faac61 |
| | /ApEinfo_fwdcolor=#faac61 |
| misc_feature | 1124..1148 |
| | /label=“oMF67” |
| | /ApEinfo_revcolor=#f58a5e |
| | /ApEinfo_fwdcolor=#f58a5e |
| misc_feature | 722..736 |
| | /label=“oMF184_2” |
| | /ApEinfo_revcolor=#9eafd2 |
| | /ApEinfo_fwdcolor=#9eafd2 |
| misc_feature | 935..935 |
| | /label=“cloning scar” |
| | /ApEinfo_revcolor=#ff9ccd |
| | /ApEinfo_fwdcolor=#ff9ccd |
| misc_feature | 1481..1508 |
| | /label=“WPRE F” |
| | /ApEinfo_revcolor=#ffef86 |
| | /ApEinfo_fwdcolor=#ffef86 |
| repeat_region | 1..106 |
| | /label=“5′-ITR” |
| | /ApEinfo_revcolor=#c6c9d1 |
| | /ApEinfo_fwdcolor=#c6c9d1 |
| misc_feature | 1823..1846 |
| | /label=“oMF253 F for ITR seq” |
| | /ApEinfo_revcolor=#b4abac |
| | /ApEinfo_fwdcolor=#b4abac |
| promoter | 428..705 |
| | /label=“chicken beta-actin promoter” |
| | /ApEinfo_revcolor=#c7b0e3 |
| | /ApEinfo_fwdcolor=#c7b0e3 |
| misc_feature | 843..934 |
| | /label=“Cbh 3′ cloning site” |
| | /ApEinfo_revcolor=#c6c9d1 |
| | /ApEinfo_fwdcolor=#c6c9d1 |
| misc_feature | 735..755 |
| | /label=“oMF203” |
| | /ApEinfo_revcolor=#d59687 |
| | /ApEinfo_fwdcolor=#d59687 |
| misc_feature | 916..934 |
| | /label=“3′ end of hybrid intron” |
| | /ApEinfo_revcolor=#b7e6d7 |
| | /ApEinfo_fwdcolor=#b7e6d7 |
| exon | 1050..1264 |
| | /label=“Cisd2 exon” |
| | /ApEinfo_revcolor=#b7e6d7 |
| | /ApEinfo_fwdcolor=#b7e6d7 |
| CDS | 947..1354 |
| | /label=“Cisd2 CDS (CDGSH iron-sulfur |
| | domain-containing protein 2)” |
| | /ApEinfo_revcolor=#faac61 |
| | /ApEinfo_fwdcolor=#faac61 |
| misc_feature | 1258..1281 |
| | /label=“oMF152 Forward” |
| | /ApEinfo_revcolor=#d6b295 |
| | /ApEinfo_fwdcolor=#d6b295 |
| misc_feature | 935..940 |
| | /label=“cloning scar” |
| | /ApEinfo_revcolor=#85dae9 |
| | /ApEinfo_fwdcolor=#85dae9 |
| misc_feature | 1058..1126 |
| | /label=“Cisd2 misc_feature” |
| | /ApEinfo_revcolor=#b1ff67 |
| | /ApEinfo_fwdcolor=#b1ff67 |
| enhancer | 123..426 |
| | /label=“CMV enhancer” |
| | /ApEinfo_revcolor=#f58a5e |
| | /ApEinfo_fwdcolor=#f58a5e |
| misc_feature | 1711..1726 |
| | /label=“BhjPa R” |
| | /ApEinfo_revcolor=#faac61 |
| | /ApEinfo_fwdcolor=#faac61 |
| misc_feature | 706..934 |
| | /label=“Hybrid intron” |
| | /ApEinfo_revcolor=#b7e6d7 |
| | /ApEinfo_fwdcolor=#b7e6d7 |
| misc_feature | 230..328 |
| | /label=“stem-loop” |
| | /ApEinfo_revcolor=#ffef86 |
| | /ApEinfo_fwdcolor=#ffef86 |
| misc_feature | 1219..1243 |
| | /label=“oMF68” |
| | /ApEinfo_revcolor=#d6b295 |
| | /ApEinfo_fwdcolor=#d6b295 |
| misc_feature | 117..121 |
| | /label=“Cbh 5′ cloning site” |
| | /ApEinfo_revcolor=#b4abac |
| | /ApEinfo_fwdcolor=#b4abac |
| repeat_region | complement(1859..1988) |
| | /label=“3′-ITR” |
| | /ApEinfo_revcolor=#ffef86 |
| | /ApEinfo_fwdcolor=#ffef86 |
| misc_feature | 719..733 |
| | /label=“oMF186_2” |
| | /ApEinfo_revcolor=#b4abac |
| | /ApEinfo_fwdcolor=#b4abac |
| misc_feature | 1531..1555 |
| | /label=“oMF80” |
| | /ApEinfo_revcolor=#faac61 |
| | /ApEinfo_fwdcolor=#faac61 |
| misc_feature | 1171..1195 |
| | /label=“oMF154-Forward” |
| | /ApEinfo_revcolor=#b4abac |
| | /ApEinfo_fwdcolor=#b4abac |
| exon | 947..1049 |
| | /label=“Cisd2 exon” |
| | /ApEinfo_revcolor=#b7e6d7 |
| | /ApEinfo_fwdcolor=#b7e6d7 |
| misc_feature | 1859..1880 |
| | /label=“D loop (Terminal resolution |
| | site based on McCarty 2004)” |
| | /ApEinfo_revcolor=#faac61 |
| | /ApEinfo_fwdcolor=#faac61 |
| misc_feature | 1397..1643 |
| | /label=“WPRE3 correct” |
| | /ApEinfo_revcolor=#f8d3a9 |
| | /ApEinfo_fwdcolor=#f8d3a9 |
| misc_feature | 123..934 |
| | /label=“Cbh” |
| | /ApEinfo_revcolor=#9eafd2 |
| | /ApEinfo_fwdcolor=#9eafd2 |
| exon | 1265..1354 |
| | /label=“Cisd2 exon” |
| | /ApEinfo_revcolor=#ff9ccd |
| | /ApEinfo_fwdcolor=#ff9ccd |
| misc_feature | 1859..1882 |
| | /label=“deleted in 5′ ITR” |
| | /ApEinfo_revcolor=#c7b0e3 |
| | /ApEinfo_fwdcolor=#c7b0e3 |
| misc_feature | 1382..1389 |
| | /label=“Seed region” |
| | /ApEinfo_revcolor=#faac61 |
| | /ApEinfo_fwdcolor=#faac61 |
| misc_feature | 1361..1390 |
| | /label=“mIR122 target site m8” |
| | /ApEinfo_revcolor=#9eafd2 |
| | /ApEinfo_fwdcolor=#9eafd2 |

ORIGIN

```
(SEQ ID NO: 13)
      1 ctgcgcgctc gctcgctcac tgaggccggg cgaccaaagg tcgcccgacg cccgggcttt
    61 gcccgggcgg cctcagtgag cgagcgagcg cgcagagagg gagtggttaa gctagcggta
  121 cccgttacat aacttacggt aaatggcccg cctggctgac cgcccaacga cccccgccca
  181 ttgacgtcaa taatgacgta tgttcccata gtaacgccaa tagggacttt ccattgacgt
  241 caatgggtgg agtatttacg gtaaactgcc cacttggcag tacatcaagt gtatcatatg
  301 ccaagtacgc cccctattga cgtcaatgac ggtaaatggc ccgcctggca ttatgcccag
  361 tacatgacct tatgggactt tcctacttgg cagtacatct acgtattagt catcgctatt
  421 accatggtcg aggtgagccc cacgttctgc ttcactctcc ccatctcccc cccctcccca
  481 cccccaattt tgtatttatt tattttttaa ttattttgtg cagcgatggg ggcggggggg
  541 gggggggggc gcgcgccagg cggggcgggg cggggcgagg ggcggggcgg ggcgaggcgg
  601 agaggtgcgg cggcagccaa tcagagcggc gcgctccgaa agtttccttt tatggcgagg
  661 cggcggcggc ggcggcccta taaaaagcga agcgcgcggc gggcgggagt cgctgcgacg
  721 ctgccttcgc cccgtgcccc gctccgccgc cgcctcgcgc cgcccgcccc ggctctgact
  781 gaccgcgtta ctcccacagg tgagcgggcg ggacggccct tctcctccgg gctgtaatta
  841 gctgagcaag aggtaagggt ttaagggatg gttggttggt ggggtattaa tgtttaatta
  901 cctggagcac ctgcctgaaa tcactttttt tcagacgcgt gccaccatgg tcctggacag
  961 cgtggcccgc atcgtgaagg tgcagctgcc cgcctacctc aagcagctcc cggtccccga
1021 cagcatcacc gggttcgccc gcctcacagt ttcagactgg ctccgcctac tgcccttcct
1081 cggggtactt gcgcttctgg gctacctcgc agtgcgccca ttcttcccaa agaagaagca
1141 acagaaggat agcttgatca atcttaagat acaaaaggaa aatcccaagg tggtgaatga
1201 gataaacatt gaagatctgt gtctcaccaa agcagcttat tgtaggtgct ggcggtccaa
1261 gacgtttcct gcctgtgatg gatcccataa taagcataat gaattgacag gcgataacgt
1321 gggtcctctc atcctgaaga agaaagaagt atagaagctt atcgcgaaca aacaccattg
1381 tcacactcca actagtataa tcaacctctg gattacaaaa tttgtgaaag attgactggt
1441 attcttaact atgttgctcc ttttacgcta tgtggatacg ctgctttaat gcctttgtat
1501 catgctattg cttcccgtat ggctttcatt ttctcctcct tgtataaatc ctggttagtt
1561 cttgccacgg cggaactcat cgccgcctgc cttgcccgct gctggacagg ggctcggctg
1621 ttgggcactg acaattccgt ggtgcctcga ctgtgccttc tagttgccag ccatctgttg
1681 tttgcccctc ccccgtgcct tccttgaccc tggaaggtgc cactcccact gtcctttcct
1741 aataaaatga ggaaattgca tcgcattgtc tgagtaggtg tcattctatt ctggggggtg
1801 gggtggggca ggacagcaag ggggaggatt gggaagacaa tagcaggcat gctggggaag
1861 gaacccctag tgatggagtt ggccactccc tctctgcgcg ctcgctcgct cactgaggcc
1921 gggcgaccaa aggtcgcccg acgcccgggc tttgcccggg cggcctcagt gagcgagcga
1981 gcgcgcagcc ttaattaacc taattcactg gccgtcgttt tacaacgtcg tgactgggaa
2041 aaccctggcg ttacccaact taatcgcctt gcagcacatc cccctttcgc cagctggcgt
2101 aatagcgaag aggcccgcac cgatcgccct tcccaacagt tgcgcagcct gaatggcgaa
2161 tgggacgcgc cctgtagcgg cgcattaagc gcggcgggtg tggtggttac gcgcagcgtg
2221 accgctacac ttgccagcgc cctagcgccc gctcctttcg ctttcttccc ttcctttctc
2281 gccacgttcg ccggctttcc ccgtcaagct ctaaatcggg ggctcccttt agggttccga
2341 tttagtgctt tacggcacct cgaccccaaa aaacttgatt agggtgatgg ttcacgtagt
2401 gggccatcgc cctgatagac ggtttttcgc cctttgacgt tggagtccac gttctttaat
2461 agtggactct tgttccaaac tggaacaaca ctcaacccta tctcggtcta ttcttttgat
2521 ttataaggga ttttgccgat ttcggcctat tggttaaaaa atgagctgat ttaacaaaaa
2581 tttaacgcga attttaacaa aatattaacg cttacaattt aggtggcact tttcggggaa
2641 atgtgcgcgg aacccctatt tgtttatttt tctaaataca ttcaaatatg tatccgctca
2701 tgagacaata accctgataa atgcttcaat aatattgaaa aaggaagagt atgagtattc
2761 aacatttccg tgtcgccctt attccctttt ttgcggcatt ttgccttcct gtttttgctc
2821 acccagaaac gctggtgaaa gtaaaagatg ctgaagatca gttgggtgca cgagtgggtt
2881 acatcgaact ggatctcaac agcggtaaga tccttgagag ttttcgcccc gaagaacgtt
2941 ttccaatgat gagcactttt aaagttctgc tatgtggcgc ggtattatcc cgtattgacg
3001 ccgggcaaga gcaactcggt cgccgcatac actattctca gaatgacttg gttgagtact
3061 caccagtcac agaaaagcat cttacggatg gcatgacagt aagagaatta tgcagtgctg
3121 ccataaccat gagtgataac actgcggcca acttacttct gacaacgatc ggaggaccga
3181 aggagctaac cgcttttttg cacaacatgg gggatcatgt aactcgcctt gatcgttggg
3241 aaccggagct gaatgaagcc ataccaaacg acgagcgtga caccacgatg cctgtagcaa
3301 tggcaacaac gttgcgcaaa ctattaactg gcgaactact tactctagct tcccggcaac
3361 aattaataga ctggatggag gcggataaag ttgcaggacc acttctgcgc tcggcccttc
3421 cggctggctg gtttattgct gataaatctg gagccggtga gcgtgggtct cgcggtatca
3481 ttgcagcact ggggccagat ggtaagccct cccgtatcgt agttatctac acgacgggga
3541 gtcaggcaac tatggatgaa cgaaatagac agatcgctga gataggtgcc tcactgatta
3601 agcattggta actgtcagac caagtttact catatatact ttagattgat ttaaaacttc
3661 atttttaatt taaaaggatc taggtgaaga tcctttttga taatctcatg accaaaatcc
3721 cttaacgtga gttttcgttc cactgagcgt cagaccccgt agaaaagatc aaaggatctt
3781 cttgagatcc tttttttctg cgcgtaatct gctgcttgca aacaaaaaaa ccaccgctac
3841 cagcggtggt ttgtttgccg gatcaagagc taccaactct ttttccgaag gtaactggct
3901 tcagcagagc gcagatacca aatactgttc ttctagtgta gccgtagtta ggccaccact
3961 tcaagaactc tgtagcaccg cctacatacc tcgctctgct aatcctgtta ccagtggctg
4021 ctgccagtgg cgataagtcg tgtcttaccg ggttggactc aagacgatag ttaccggata
4081 aggcgcagcg gtcgggctga acggggggtt cgtgcacaca gcccagcttg gagcgaacga
4141 cctacaccga actgagatac ctacagcgtg agctatgaga aagcgccacg cttcccgaag
4201 ggagaaaggc ggacaggtat ccggtaagcg gcagggtcgg aacaggagag cgcacgaggg
4261 agcttccagg gggaaacgcc tggtatcttt atagtcctgt cgggtttcgc cacctctgac
4321 ttgagcgtcg atttttgtga tgctcgtcag gggggcggag cctatggaaa aacgccagca
4381 acgcggcctt tttacggttc ctggcctttt gctggccttt tgctcacatg ttctttcctg
4441 cgttatcccc tgattctgtg gataaccgta ttaccgcctt tgagtgagct gataccgctc
4501 gccgcagccg aacgaccgag cgcagcgagt cagtgagcga ggaagcggaa gagcgcccaa
4561 tacgcaaacc gcctctcccc gcgcgttggc cgattcatta atgcagctgg cacgacaggt
4621 ttcccgactg gaaagcgggc agtgagcgca acgcaattaa tgtgagttag ctcactcatt
4681 aggcacccca ggctttacac tttatgcttc cggctcgtat gttgtgtgga attgtgagcg
4741 gataacaatt tcacacagga aacagctatg accatgatta cgccagattt aattaaggga
4801 tctgggccac tccctct
```

LOCUS scAAV-CbhM-GFP-miR122-8 5129 bp ds-DNA circular DEFINITION

| | |
|---|---|
| FEATURES | Location/Qualifiers |
| CDS | 3829..4689 |
| | /label=“Amp-R” |
| | /ApEinfo_revcolor=#f58a5e |
| | /ApEinfo_fwdcolor=#f58a5e |
| misc_feature | 2460..2467 |
| | /label=“Seed region” |
| | /ApEinfo_revcolor=#faac61 |
| | /ApEinfo_fwdcolor=#faac61 |
| promoter | 1194..1471 |
| | /label=“chicken beta-actin promoter” |
| | /ApEinfo_revcolor=#c7b0e3 |
| | /ApEinfo_fwdcolor=#c7b0e3 |
| misc_feature | 1707..1712 |
| | /label=“Kozak” |
| | /ApEinfo_revcolor=#ff9ccd |
| | /ApEinfo_fwdcolor=#ff9ccd |
| misc_feature | 1682..1700 |
| | /label=“3′ end of hybrid intron” |
| | /ApEinfo_revcolor=#b7e6d7 |
| | /ApEinfo_fwdcolor=#b7e6d7 |
| misc_feature | 883..887 |
| | /label=“Cbh 5′ cloning site” |
| | /ApEinfo_revcolor=#b4abac |
| | /ApEinfo_fwdcolor=#b4abac |
| misc_feature | 1609..1700 |
| | /label=“Cbh 3′ cloning site” |
| | /ApEinfo_revcolor=#c6c9d1 |
| | /ApEinfo_fwdcolor=#c6c9d1 |
| misc_feature | 2937..2960 |
| | /label=“deleted in 5′ ITR” |
| | /ApEinfo_revcolor=#c7b0e3 |
| | /ApEinfo_fwdcolor=#c7b0e3 |
| misc_feature | 2439..2468 |
| | /label=“mIR122 target site m8” |
| | /ApEinfo_revcolor=#9eafd2 |
| | /ApEinfo_fwdcolor=#9eafd2 |
| polyA_signal | 2722..2936 |
| | /label=“BGH\pA” |
| | /ApEinfo_revcolor=#faac61 |
| | /ApEinfo_fwdcolor=#faac61 |
| misc_feature | 2475..2721 |
| | /label=“WPRE3 correct” |
| | /ApEinfo_revcolor=#f8d3a9 |
| | /ApEinfo_fwdcolor=#f8d3a9 |
| misc_feature | 2609..2633 |
| | /label=“oMF80” |
| | /ApEinfo_revcolor=#faac61 |
| | /ApEinfo_fwdcolor=#faac61 |
| enhancer | 889..1192 |
| | /label=“CMV enhancer” |
| | /ApEinfo_revcolor=#f58a5e |
| | /ApEinfo_fwdcolor=#f58a5e |
| repeat_region | complement(2937..3066) |
| | /label=“3′-ITR” |
| | /ApEinfo_revcolor=#ffef86 |
| | /ApEinfo_fwdcolor=#ffef86 |
| misc_feature | 1701..1706 |
| | /label=“cloning scar” |
| | /ApEinfo_revcolor=#85dae9 |
| | /ApEinfo_fwdcolor=#85dae9 |
| CDS | 1713..2429 |
| | /label=“eGFP” |
| | /ApEinfo_revcolor=#75c6a9 |
| | /ApEinfo_fwdcolor=#75c6a9 |
| misc_feature | 1701..1701 |
| | /label=“cloning scar” |
| | /ApEinfo_revcolor=#ff9ccd |
| | /ApEinfo_fwdcolor=#ff9ccd |
| misc_feature | 1472..1700 |
| | /label=“Hybrid intron” |
| | /ApEinfo_revcolor=#b7e6d7 |
| | /ApEinfo_fwdcolor=#b7e6d7 |
| misc_feature | 889..1700 |
| | /label=“Cbh” |
| | /ApEinfo_revcolor=#84b0dc |
| | /ApEinfo_fwdcolor=#84b0dc |
| repeat_region | 767..872 |
| | /label=“5′-ITR” |
| | /ApEinfo_revcolor=#c6c9d1 |
| | /ApEinfo_fwdcolor=#c6c9d1 |

ORIGIN

```
(SEQ ID NO: 14)
      1 gttggactca agacgatagt taccggataa ggcgcagcgg tcgggctgaa cggggggttc
    61 gtgcacacag cccagcttgg agcgaacgac ctacaccgaa ctgagatacc tacagcgtga
  121 gctatgagaa agcgccacgc ttcccgaagg gagaaaggcg gacaggtatc cggtaagcgg
  181 cagggtcgga acaggagagc gcacgaggga gcttccaggg ggaaacgcct ggtatcttta
  241 tagtcctgtc gggtttcgcc acctctgact tgagcgtcga tttttgtgat gctcgtcagg
  301 ggggcggagc ctatggaaaa acgccagcaa cgcggccttt ttacggttcc tggccttttg
  361 ctggcctttt gctcacatgt tctttcctgc gttatcccct gattctgtgg ataaccgtat
  421 taccgccttt gagtgagctg ataccgctcg ccgcagccga acgaccgagc gcagcgagtc
  481 agtgagcgag gaagcggaag agcgcccaat acgcaaaccg cctctccccg cgcgttggcc
  541 gattcattaa tgcagctggc acgacaggtt tcccgactgg aaagcgggca gtgagcgcaa
  601 cgcaattaat gtgagttagc tcactcatta ggcaccccag gctttacact ttatgcttcc
  661 ggctcgtatg ttgtgtggaa ttgtgagcgg ataacaattt cacacaggaa acagctatga
  721 ccatgattac gccagattta attaagggat ctgggccact ccctctctgc gcgctcgctc
  781 gctcactgag gccgggcgac caaaggtcgc ccgacgcccg ggctttgccc gggcggcctc
  841 agtgagcgag cgagcgcgca gagagggagt ggttaagcta gcggtacccg ttacataact
  901 tacggtaaat ggcccgcctg gctgaccgcc caacgacccc cgcccattga cgtcaataat
  961 gacgtatgtt cccatagtaa cgccaatagg gactttccat tgacgtcaat gggtggagta
1021 tttacggtaa actgcccact tggcagtaca tcaagtgtat catatgccaa gtacgccccc
1081 tattgacgtc aatgacggta aatggcccgc ctggcattat gcccagtaca tgaccttatg
1141 ggactttcct acttggcagt acatctacgt attagtcatc gctattacca tggtcgaggt
1201 gagccccacg ttctgcttca ctctccccat ctcccccccc tccccacccc caattttgta
1261 tttatttatt ttttaattat tttgtgcagc gatgggggcg gggggggggg gggggcgcgc
1321 gccaggcggg gcggggcggg gcgaggggcg gggcggggcg aggcggagag gtgcggcggc
1381 agccaatcag agcggcgcgc tccgaaagtt tccttttatg gcgaggcggc ggcggcggcg
1441 gccctataaa aagcgaagcg cgcggcgggc gggagtcgct gcgacgctgc cttcgccccg
1501 tgccccgctc cgccgccgcc tcgcgccgcc cgccccggct ctgactgacc gcgttactcc
1561 cacaggtgag cgggcgggac ggcccttctc ctccgggctg taattagctg agcaagaggt
1621 aagggtttaa gggatggttg gttggtgggg tattaatgtt taattacctg gagcacctgc
1681 ctgaaatcac tttttttcag acgcgtgcca ccatggtgag caagggcgag gagctgttca
1741 ccggggtggt gcccatcctg gtcgagctgg acggcgacgt aaacggccac aagttcagcg
1801 tgtccggcga gggcgagggc gatgccacct acggcaagct gaccctgaag ttcatctgca
1861 ccaccggcaa gctgcccgtg ccctggccca ccctcgtgac caccctgacc tacggcgtgc
1921 agtgcttcag ccgctacccc gaccacatga agcagcacga cttcttcaag tccgccatgc
1981 ccgaaggcta cgtccaggag cgcaccatct tcttcaagga cgacggcaac tacaagaccc
2041 gcgccgaggt gaagttcgag ggcgacaccc tggtgaaccg catcgagctg aagggcatcg
2101 acttcaagga ggacggcaac atcctggggc acaagctgga gtacaactac aacagccaca
2161 acgtctatat catggccgac aagcagaaga acggcatcaa ggtgaacttc aagatccgcc
2221 acaacatcga ggacggcagc gtgcagctcg ccgaccacta ccagcagaac acccccatcg
2281 gcgacggccc cgtgctgctg cccgacaacc actacctgag cacccagtcc gccctgagca
2341 aagaccccaa cgagaagcgc gatcacatgg tcctgctgga gttcgtgacc gccgccggga
2401 tcactctcgg catggacgag ctgtacaagt aaaagcttat cgcgaacaaa caccattgtc
2461 acactccaac tagtataatc aacctctgga ttacaaaatt tgtgaaagat tgactggtat
2521 tcttaactat gttgctcctt ttacgctatg tggatacgct gctttaatgc ctttgtatca
2581 tgctattgct tcccgtatgg ctttcatttt ctcctccttg tataaatcct ggttagttct
2641 tgccacggcg gaactcatcg ccgcctgcct tgcccgctgc tggacagggg ctcggctgtt
2701 gggcactgac aattccgtgg tgcctcgact gtgccttcta gttgccagcc atctgttgtt
2761 tgcccctccc ccgtgccttc cttgaccctg gaaggtgcca ctcccactgt cctttcctaa
2821 taaaatgagg aaattgcatc gcattgtctg agtaggtgtc attctattct ggggggtggg
2881 gtggggcagg acagcaaggg ggaggattgg gaagacaata gcaggcatgc tggggaagga
2941 acccctagtg atggagttgg ccactccctc tctgcgcgct cgctcgctca ctgaggccgg
3001 gcgaccaaag gtcgcccgac gcccgggctt tgcccgggcg gcctcagtga gcgagcgagc
3061 gcgcagcctt aattaaccta attcactggc cgtcgtttta caacgtcgtg actgggaaaa
3121 ccctggcgtt acccaactta atcgccttgc agcacatccc cctttcgcca gctggcgtaa
3181 tagcgaagag gcccgcaccg atcgcccttc ccaacagttg cgcagcctga atggcgaatg
3241 ggacgcgccc tgtagcggcg cattaagcgc ggcgggtgtg gtggttacgc gcagcgtgac
3301 cgctacactt gccagcgccc tagcgcccgc tcctttcgct ttcttccctt cctttctcgc
3361 cacgttcgcc ggctttcccc gtcaagctct aaatcggggg ctccctttag ggttccgatt
3421 tagtgcttta cggcacctcg accccaaaaa acttgattag ggtgatggtt cacgtagtgg
3481 gccatcgccc tgatagacgg tttttcgccc tttgacgttg gagtccacgt tctttaatag
3541 tggactcttg ttccaaactg gaacaacact caaccctatc tcggtctatt cttttgattt
3601 ataagggatt ttgccgattt cggcctattg gttaaaaaat gagctgattt aacaaaaatt
3661 taacgcgaat tttaacaaaa tattaacgct tacaatttag gtggcacttt tcggggaaat
3721 gtgcgcggaa cccctatttg tttatttttc taaatacatt caaatatgta tccgctcatg
3781 agacaataac cctgataaat gcttcaataa tattgaaaaa ggaagagtat gagtattcaa
3841 catttccgtg tcgcccttat tccctttttt gcggcatttt gccttcctgt ttttgctcac
3901 ccagaaacgc tggtgaaagt aaaagatgct gaagatcagt tgggtgcacg agtgggttac
3961 atcgaactgg atctcaacag cggtaagatc cttgagagtt ttcgccccga agaacgtttt
4021 ccaatgatga gcacttttaa agttctgcta tgtggcgcgg tattatcccg tattgacgcc
4081 gggcaagagc aactcggtcg ccgcatacac tattctcaga atgacttggt tgagtactca
4141 ccagtcacag aaaagcatct tacggatggc atgacagtaa gagaattatg cagtgctgcc
4201 ataaccatga gtgataacac tgcggccaac ttacttctga caacgatcgg aggaccgaag
4261 gagctaaccg cttttttgca caacatgggg gatcatgtaa ctcgccttga tcgttgggaa
4321 ccggagctga atgaagccat accaaacgac gagcgtgaca ccacgatgcc tgtagcaatg
4381 gcaacaacgt tgcgcaaact attaactggc gaactactta ctctagcttc ccggcaacaa
4441 ttaatagact ggatggaggc ggataaagtt gcaggaccac ttctgcgctc ggcccttccg
4501 gctggctggt ttattgctga taaatctgga gccggtgagc gtgggtctcg cggtatcatt
4561 gcagcactgg ggccagatgg taagccctcc cgtatcgtag ttatctacac gacggggagt
4621 caggcaacta tggatgaacg aaatagacag atcgctgaga taggtgcctc actgattaag
4681 cattggtaac tgtcagacca agtttactca tatatacttt agattgattt aaaacttcat
4741 ttttaattta aaaggatcta ggtgaagatc ctttttgata atctcatgac caaaatccct
4801 taacgtgagt tttcgttcca ctgagcgtca gaccccgtag aaaagatcaa aggatcttct
4861 tgagatcctt tttttctgcg cgtaatctgc tgcttgcaaa caaaaaaacc accgctacca
4921 gcggtggttt gtttgccgga tcaagagcta ccaactcttt ttccgaaggt aactggcttc
4981 agcagagcgc agataccaaa tactgttctt ctagtgtagc cgtagttagg ccaccacttc
5041 aagaactctg tagcaccgcc tacatacctc gctctgctaa tcctgttacc agtggctgct
5101 gccagtggcg ataagtcgtg tcttaccgg
```

LOCUS scAAV-CbhM-PTEN-miR122-5283 bp ds-DNA circular DEFINITION

| | |
|---|---|
| FEATURES | Location/Qualifiers |
| misc_feature | 1701..1706 |
| | /label=“cloning scar” |
| | /ApEinfo_revcolor=#85dae9 |
| | /ApEinfo_fwdcolor=#85dae9 |
| misc_feature | 1713..2924 |
| | /label=“mPTEN” |
| | /ApEinfo_revcolor=#b4abac |
| | /ApEinfo_fwdcolor=#b4abac |
| misc_feature | 1682..1700 |
| | /label=“3′ end of hybrid intron” |
| | /ApEinfo_revcolor=#b7e6d7 |
| | /ApEinfo_fwdcolor=#b7e6d7 |
| misc_feature | 1609..1700 |
| | /label=“Cbh 3′ cloning site” |
| | /ApEinfo_revcolor=#c6c9d1 |
| | /ApEinfo_fwdcolor=#c6c9d1 |
| CDS | 3983..4843 |
| | /label=“Amp-R” |
| | /ApEinfo_revcolor=#f58a5e |
| | /ApEinfo_fwdcolor=#f58a5e |
| misc_feature | 2934..2963 |
| | /label=“mIR122 target site m8” |
| | /ApEinfo_revcolor=#9eafd2 |
| | /ApEinfo_fwdcolor=#9eafd2 |
| misc_feature | 1472..1700 |
| | /label=“Hybrid intron” |
| | /ApEinfo_revcolor=#b7e6d7 |
| | /ApEinfo_fwdcolor=#b7e6d7 |
| promoter | 1194..1471 |
| | /label=“chicken beta-actin promoter” |
| | /ApEinfo_revcolor=#c7b0e3 |
| | /ApEinfo_fwdcolor=#c7b0e3 |
| repeat_region | 767..872 |
| | /label=“5′-ITR” |
| | /ApEinfo_revcolor=#c6c9d1 |
| | /ApEinfo_fwdcolor=#c6c9d1 |
| enhancer | 889..1192 |
| | /label=“CMV enhancer” |
| | /ApEinfo_revcolor=#f58a5e |
| | /ApEinfo_fwdcolor=#f58a5e |
| misc_feature | 1701..1701 |
| | /label=“cloning scar” |
| | /ApEinfo_revcolor=#ff9ccd |
| | /ApEinfo_fwdcolor=#ff9ccd |
| misc_feature | 1707..1712 |
| | /label=“Kozak” |
| | /ApEinfo_revcolor=#ff9ccd |
| | /ApEinfo_fwdcolor=#ff9ccd |
| misc_feature | 2970..3090 |
| | /label=“SV40pA” |
| | /ApEinfo_revcolor=#d59687 |
| | /ApEinfo_fwdcolor=#d59687 |
| misc_feature | 3091..3114 |
| | /label=“deleted in 5′ ITR” |
| | /ApEinfo_revcolor=#c7b0e3 |
| | /ApEinfo_fwdcolor=#c7b0e3 |
| repeat_region | complement(3091..3220) |
| | /label=“3′-ITR” |
| | /ApEinfo_revcolor=#ffef86 |
| | /ApEinfo_fwdcolor=#ffef86 |
| misc_feature | 889..1700 |
| | /label=“Cbh” |
| | /ApEinfo_revcolor=#84b0dc |
| | /ApEinfo_fwdcolor=#84b0dc |
| misc_feature | 883..887 |
| | /label=“Cbh 5′ cloning site” |
| | /ApEinfo_revcolor=#b4abac |
| | /ApEinfo_fwdcolor=#b4abac |
| misc_feature | 2955..2962 |
| | /label=“Seed region” |
| | /ApEinfo_revcolor=#faac61 |
| | /ApEinfo_fwdcolor=#faac61 |

ORIGIN

```
(SEQ ID NO: 15)
      1 gttggactca agacgatagt taccggataa ggcgcagcgg tcgggctgaa cggggggttc
    61 gtgcacacag cccagcttgg agcgaacgac ctacaccgaa ctgagatacc tacagcgtga
  121 gctatgagaa agcgccacgc ttcccgaagg gagaaaggcg gacaggtatc cggtaagcgg
  181 cagggtcgga acaggagagc gcacgaggga gcttccaggg ggaaacgcct ggtatcttta
  241 tagtcctgtc gggtttcgcc acctctgact tgagcgtcga tttttgtgat gctcgtcagg
  301 ggggcggagc ctatggaaaa acgccagcaa cgcggccttt ttacggttcc tggccttttg
  361 ctggcctttt gctcacatgt tctttcctgc gttatcccct gattctgtgg ataaccgtat
  421 taccgccttt gagtgagctg ataccgctcg ccgcagccga acgaccgagc gcagcgagtc
  481 agtgagcgag gaagcggaag agcgcccaat acgcaaaccg cctctccccg cgcgttggcc
  541 gattcattaa tgcagctggc acgacaggtt tcccgactgg aaagcgggca gtgagcgcaa
  601 cgcaattaat gtgagttagc tcactcatta ggcaccccag gctttacact ttatgcttcc
  661 ggctcgtatg ttgtgtggaa ttgtgagcgg ataacaattt cacacaggaa acagctatga
  721 ccatgattac gccagattta attaagggat ctgggccact ccctctctgc gcgctcgctc
  781 gctcactgag gccgggcgac caaaggtcgc ccgacgcccg ggctttgccc gggcggcctc
  841 agtgagcgag cgagcgcgca gagagggagt ggttaagcta gcggtacccg ttacataact
  901 tacggtaaat ggcccgcctg gctgaccgcc caacgacccc cgcccattga cgtcaataat
  961 gacgtatgtt cccatagtaa cgccaatagg gactttccat tgacgtcaat gggtggagta
1021 tttacggtaa actgcccact tggcagtaca tcaagtgtat catatgccaa gtacgccccc
1081 tattgacgtc aatgacggta aatggcccgc ctggcattat gcccagtaca tgaccttatg
1141 ggactttcct acttggcagt acatctacgt attagtcatc gctattacca tggtcgaggt
1201 gagccccacg ttctgcttca ctctccccat ctcccccccc tccccacccc caattttgta
1261 tttatttatt ttttaattat tttgtgcagc gatgggggcg gggggggggg gggggcgcgc
1321 gccaggcggg gcggggcggg gcgaggggcg gggcggggcg aggcggagag gtgcggcggc
1381 agccaatcag agcggcgcgc tccgaaagtt tccttttatg gcgaggcggc ggcggcggcg
1441 gccctataaa aagcgaagcg cgcggcgggc gggagtcgct gcgacgctgc cttcgccccg
1501 tgccccgctc cgccgccgcc tcgcgccgcc cgccccggct ctgactgacc gcgttactcc
1561 cacaggtgag cgggcgggac ggcccttctc ctccgggctg taattagctg agcaagaggt
1621 aagggtttaa gggatggttg gttggtgggg tattaatgtt taattacctg gagcacctgc
1681 ctgaaatcac tttttttcag acgcgtgcca ccatgacagc catcatcaaa gagatcgtta
1741 gcagaaacaa aaggagatat caagaggatg gattcgactt agacttgacc tatatttatc
1801 caaatattat tgctatggga tttcctgcag aaagacttga aggtgtatac aggaacaata
1861 ttgatgatgt agtaaggttt ttggattcaa agcataaaaa ccattacaag atatacaatc
1921 tatgtgctga gagacattat gacaccgcca aatttaactg cagagttgca cagtatcctt
1981 ttgaagacca taacccacca cagctagaac ttatcaaacc cttctgtgaa gatcttgacc
2041 aatggctaag tgaagatgac aatcatgttg cagcaattca ctgtaaagct ggaaagggac
2101 ggactggtgt aatgatttgt gcatatttat tgcatcgggg caaattttta aaggcacaag
2161 aggccctaga tttttatggg gaagtaagga ccagagacaa aaagggagtc acaattccca
2221 gtcagaggcg ctatgtatat tattatagct acctgctaaa aaatcacctg gattacagac
2281 ccgtggcact gctgtttcac aagatgatgt ttgaaactat tccaatgttc agtggcggaa
2341 cttgcaatcc tcagtttgtg gtctgccagc taaaggtgaa gatatattcc tccaattcag
2401 gacccacgcg gcgggaggac aagttcatgt actttgagtt ccctcagcca ttgcctgtgt
2461 gtggtgatat caaagtagag ttcttccaca aacagaacaa gatgctcaaa aaggacaaaa
2521 tgtttcactt ttgggtaaat acgttcttca taccaggacc agaggaaacc tcagaaaaag
2581 tggaaaatgg aagtctttgt gatcaggaaa tcgatagcat ttgcagtata gagcgtgcag
2641 ataatgacaa ggagtatctt gtactcaccc taacaaaaaa cgatcttgac aaagcaaaca
2701 aagacaaggc caaccgatac ttctctccaa attttaaggt gaaactatac tttacaaaaa
2761 cagtagagga gccatcaaat ccagaggcta gcagttcaac ttctgtgact ccagatgtta
2821 gtgacaatga acctgatcat tatagatatt ctgacaccac tgactctgat ccagagaatg
2881 aaccttttga tgaagatcag cattcacaaa ttacaaaagt ctgataaaag cttatcgcga
2941 acaaacacca ttgtcacact ccaactagtt aagatacatt gatgagtttg gacaaaccac
3001 aactagaatg cagtgaaaaa aatgctttat ttgtgaaatt tgtgatgcta ttgctttatt
3061 tgtaaccatt ataagctgca ataaacaagt aggaacccct agtgatggag ttggccactc
3121 cctctctgcg cgctcgctcg ctcactgagg ccgggcgacc aaaggtcgcc cgacgcccgg
3181 gctttgcccg ggcggcctca gtgagcgagc gagcgcgcag ccttaattaa cctaattcac
3241 tggccgtcgt tttacaacgt cgtgactggg aaaaccctgg cgttacccaa cttaatcgcc
3301 ttgcagcaca tccccctttc gccagctggc gtaatagcga agaggcccgc accgatcgcc
3361 cttcccaaca gttgcgcagc ctgaatggcg aatgggacgc gccctgtagc ggcgcattaa
3421 gcgcggcggg tgtggtggtt acgcgcagcg tgaccgctac acttgccagc gccctagcgc
3481 ccgctccttt cgctttcttc ccttcctttc tcgccacgtt cgccggcttt ccccgtcaag
3541 ctctaaatcg ggggctccct ttagggttcc gatttagtgc tttacggcac ctcgacccca
3601 aaaaacttga ttagggtgat ggttcacgta gtgggccatc gccctgatag acggtttttc
3661 gccctttgac gttggagtcc acgttcttta atagtggact cttgttccaa actggaacaa
3721 cactcaaccc tatctcggtc tattcttttg atttataagg gattttgccg atttcggcct
3781 attggttaaa aaatgagctg atttaacaaa aatttaacgc gaattttaac aaaatattaa
3841 cgcttacaat ttaggtggca cttttcgggg aaatgtgcgc ggaaccccta tttgtttatt
3901 tttctaaata cattcaaata tgtatccgct catgagacaa taaccctgat aaatgcttca
3961 ataatattga aaaaggaaga gtatgagtat tcaacatttc cgtgtcgccc ttattccctt
4021 ttttgcggca ttttgccttc ctgtttttgc tcacccagaa acgctggtga aagtaaaaga
4081 tgctgaagat cagttgggtg cacgagtggg ttacatcgaa ctggatctca acagcggtaa
4141 gatccttgag agttttcgcc ccgaagaacg ttttccaatg atgagcactt ttaaagttct
4201 gctatgtggc gcggtattat cccgtattga cgccgggcaa gagcaactcg gtcgccgcat
4261 acactattct cagaatgact tggttgagta ctcaccagtc acagaaaagc atcttacgga
4321 tggcatgaca gtaagagaat tatgcagtgc tgccataacc atgagtgata acactgcggc
4381 caacttactt ctgacaacga tcggaggacc gaaggagcta accgcttttt tgcacaacat
4441 gggggatcat gtaactcgcc ttgatcgttg ggaaccggag ctgaatgaag ccataccaaa
4501 cgacgagcgt gacaccacga tgcctgtagc aatggcaaca acgttgcgca aactattaac
4561 tggcgaacta cttactctag cttcccggca acaattaata gactggatgg aggcggataa
4621 agttgcagga ccacttctgc gctcggccct tccggctggc tggtttattg ctgataaatc
4681 tggagccggt gagcgtgggt ctcgcggtat cattgcagca ctggggccag atggtaagcc
4741 ctcccgtatc gtagttatct acacgacggg gagtcaggca actatggatg aacgaaatag
4801 acagatcgct gagataggtg cctcactgat taagcattgg taactgtcag accaagttta
4861 ctcatatata ctttagattg atttaaaact tcatttttaa tttaaaagga tctaggtgaa
4921 gatccttttt gataatctca tgaccaaaat cccttaacgt gagttttcgt tccactgagc
4981 gtcagacccc gtagaaaaga tcaaaggatc ttcttgagat cctttttttc tgcgcgtaat
5041 ctgctgcttg caaacaaaaa aaccaccgct accagcggtg gtttgtttgc cggatcaaga
5101 gctaccaact ctttttccga aggtaactgg cttcagcaga gcgcagatac caaatactgt
5161 tcttctagtg tagccgtagt taggccacca cttcaagaac tctgtagcac cgcctacata
5221 cctcgctctg ctaatcctgt taccagtggc tgctgccagt ggcgataagt cgtgtcttac
5281 cgg
```

LOCUS AAV_pCAG-Atg5-WPRE-bGHp 6325 bp ds-DNA circular DEFINITION
KEYWORDS “accession:addgene_65455_110978”

| FEATURES | Location/Qualifiers |
|---|---|
| misc_feature | 2957..3050 |
| | /label=“Chicken beta actin exon 1 full” |
| | /ApEinfo_revcolor=#ff9ccd |
| | /ApEinfo_fwdcolor=#ff9ccd |
| misc_feature | 2622..2643 |
| | /label=“o2MF1 primer binding site” |
| | /ApEinfo_revcolor=#75c6a9 |
| | /ApEinfo_fwdcolor=#75c6a9 |
| misc_feature | 2930..2956 |
| | /label=“oMF209” |
| | /ApEinfo_revcolor=#c7b0e3 |
| | /ApEinfo_fwdcolor=#c7b0e3 |
| misc_feature | 4064..4088 |
| | /label=“oMF65” |
| | /ApEinfo_revcolor=#d6b295 |
| | /ApEinfo_fwdcolor=#d6b295 |
| misc_feature | 1267..1295 |
| | /label=“AmpR promtoer” |
| | /ApEinfo_revcolor=#85dae9 |
| | /ApEinfo_fwdcolor=#85dae9 |
| misc_feature | 2958..4027 |
| | /label=“chicken beta actin |
| | exon-intron-rabbit beta globin intron” |
| | /ApEinfo_revcolor=#9eafd2 |
| | /ApEinfo_fwdcolor=#9eafd2 |
| STS | 4415..4532 |
| | /label=“Atg5 STS” |
| | /ApEinfo_revcolor=#c6c9d1 |
| | /ApEinfo_fwdcolor=#c6c9d1 |
| misc_feature | 2970..2984 |
| | /label=“oMF186 CAGex1_2F” |
| | /ApEinfo_revcolor=#75c6a9 |
| | /ApEinfo_fwdcolor=#75c6a9 |
| misc_feature | 4040..4042 |
| | /label=“Atg5 misc_feature” |
| | /ApEinfo_revcolor=#faac61 |
| | /ApEinfo_fwdcolor=#faac61 |
| misc_feature | 4819..4852 |
| | /label=“oMF81” |
| | /ApEinfo_revcolor=#f58a5e |
| | /ApEinfo_fwdcolor=#f58a5e |
| misc_feature | 3148..3163 |
| | /label=“oMF207” |
| | /ApEinfo_revcolor=#9eafd2 |
| | /ApEinfo_fwdcolor=#9eafd2 |
| misc_feature | 2680..2956 |
| | /label=“Chicken beta actin promoter” |
| | /ApEinfo_revcolor=#c7b0e3 |
| | /ApEinfo_fwdcolor=#c7b0e3 |
| misc_feature | 3934..4027 |
| | /label=“Rabbit beta-globin” |
| | /ApEinfo_revcolor=#b7e6d7 |
| | /ApEinfo_fwdcolor=#b7e6d7 |
| exon | 4518..4612 |
| | /label=“Atg5 exon” |
| | /ApEinfo_revcolor=#c6c9d1 |
| | /ApEinfo_fwdcolor=#c6c9d1 |
| misc_feature | 4034..4039 |
| | /label=“Kozak” |
| | /ApEinfo_revcolor=#ff9ccd |
| | /ApEinfo_fwdcolor=#ff9ccd |
| feature | 4882..5469 |
| | /label=“WPRE” |
| | /ApEinfo_revcolor=#b1ff67 |
| | /ApEinfo_fwdcolor=#b1ff67 |
| exon | 4613..4730 |
| | /label=“Atg5 exon” |
| | /ApEinfo_revcolor=#d6b295 |
| | /ApEinfo_fwdcolor=#d6b295 |
| primer | complement(5503..5520) |
| | /label=“BGH_rev_primer” |
| | /ApEinfo_revcolor=#c7b0e3 |
| | /ApEinfo_fwdcolor=#c7b0e3 |
| misc_feature | 1744..2050 |
| | /label=“F1 origin” |
| | /ApEinfo_revcolor=#85dae9 |
| | /ApEinfo_fwdcolor=#85dae9 |
| misc_feature | 5739..5869 |
| | /label=“AAV2 ITR” |
| | /ApEinfo_revcolor=#ffef86 |
| | /ApEinfo_fwdcolor=#ffef86 |
| STS | 4754.4867 |
| | /label=“Atg5 STS” |
| | /ApEinfo_revcolor=#84b0dc |
| | /ApEinfo_fwdcolor=#84b0dc |
| misc_feature | 4040..4061 |
| | /label=“oMF210-213” |
| | /ApEinfo_revcolor=#85dae9 |
| | /ApEinfo_fwdcolor=#85dae9 |
| exon | 4040..4147 |
| | /label=“Atg5 exon” |
| | /ApEinfo_revcolor=#75c6a9 |
| | /ApEinfo_fwdcolor=#75c6a9 |
| misc_feature | 3926..3933 |
| | /label=“cloning scar” |
| | /ApEinfo_revcolor=#f58a5e |
| | /ApEinfo_fwdcolor=#f58a5e |
| exon | 4276..4354 |
| | /label=“Atg5 exon” |
| | /ApEinfo_revcolor=#ffef86 |
| | /ApEinfo_fwdcolor=#ffef86 |
| misc_feature | 1425..1476 |
| | /label=“Sequence missing in |
| | original pCAG backbone” |
| | /ApEinfo_revcolor=#b1ff67 |
| | /ApEinfo_fwdcolor=#b1ff67 |
| misc_feature | 3027..3045 |
| | /label=“oMF187-CAGex1_2R” |
| | /ApEinfo_revcolor=#85dae9 |
| | /ApEinfo_fwdcolor=#85dae9 |
| misc_feature | 4772..4799 |
| | /label=“oMF 159 probe?” |
| | /ApEinfo_revcolor=#d6b295 |
| | /ApEinfo_fwdcolor=#d6b295 |
| CDS | 4040..4867 |
| | /label=“Atg5 CDS (autophagy |
| | protein 5 isoform 1)” |
| | /ApEinfo_revcolor=#c7b0e3 |
| | /ApEinfo_fwdcolor=#c7b0e3 |
| misc_feature | 4804..4823 |
| | /label=“oMF 158 Reverse” |
| | /ApEinfo_revcolor=#9eafd2 |
| | /ApEinfo_fwdcolor=#9eafd2 |
| misc_feature | 365..1225 |
| | /label=“Ampicillin” |
| | /ApEinfo_revcolor=#d59687 |
| | /ApEinfo_fwdcolor=#d59687 |
| misc_feature | 4001..4027 |
| | /label=“o2MF2 R binding site” |
| | /ApEinfo_revcolor=#b1ff67 |
| | /ApEinfo_fwdcolor=#b1ff67 |
| misc_feature | 5093..5112 |
| | /label=“o2MF14 WPRE rev primer” |
| | /ApEinfo_revcolor=#b7e6d7 |
| | /ApEinfo_fwdcolor=#b7e6d7 |
| misc_feature | 4745..4764 |
| | /label=“oMF157” |
| | /ApEinfo_revcolor=#d59687 |
| | /ApEinfo_fwdcolor=#d59687 |
| misc_feature | 4001..4027 |
| | /label=“o2MF2 R binding site” |
| | /ApEinfo_revcolor=#f8d3a9 |
| | /ApEinfo_fwdcolor=#f8d3a9 |
| misc_feature | join(5916..6325,1..210) |
| | /label=“pBR322 origin” |
| | /ApEinfo_revcolor=#ff9ccd |
| | /ApEinfo_fwdcolor=#ff9ccd |
| primer | 3982..4001 |
| | /label=“pCAG_F_primer” |
| | /ApEinfo_revcolor=#b4abac |
| | /ApEinfo_fwdcolor=#b4abac |
| misc_feature | 4180..4204 |
| | /label=“oMF150” |
| | /ApEinfo_revcolor=#b4abac |
| | /ApEinfo_fwdcolor=#b4abac |
| misc_feature | 3051..3925 |
| | /label=“chicken beta actin intron 1 5′ (some |
| SNPs compared to ENSEMBL)” | |
| | /ApEinfo_revcolor=#faac61 |
| | /ApEinfo_fwdcolor=#faac61 |
| misc_feature | join(5870..6325,1..2141) |
| | /label=“AAV-CRE inverted backbone” |
| | /ApEinfo_revcolor=#75c6a9 |
| | /ApEinfo_fwdcolor=#75c6a9 |
| misc_feature | 3772..3789 |
| | /label=“o2MF13 primer” |
| | /ApEinfo_revcolor=#75c6a9 |
| | /ApEinfo_fwdcolor=#75c6a9 |
| exon | 4355..4517 |
| | /label=“Atg5 exon” |
| | /ApEinfo_revcolor=#b7e6d7 |
| | /ApEinfo_fwdcolor=#b7e6d7 |
| misc_feature | 2968..2982 |
| | /label=“oMF204-206” |
| | /ApEinfo_revcolor=#b1ff67 |
| | /ApEinfo_fwdcolor=#b1ff67 |
| regulatory | 2379..2666 |
| | /label=“CMV enhancer” |
| | /ApEinfo_revcolor=#d59687 |
| | /ApEinfo_fwdcolor=#d59687 |
| misc_feature | 2674..2692 |
| | /label=“208” |
| | /ApEinfo_revcolor=#c7b0e3 |
| | /ApEinfo_fwdcolor=#c7b0e3 |
| misc_feature | 4161..4185 |
| | /label=“oMF149” |
| | /ApEinfo_revcolor=#ffef86 |
| | /ApEinfo_fwdcolor=#ffef86 |
| misc_feature | 4070..4093 |
| | /label=“oMF214” |
| | /ApEinfo_revcolor=#84b0dc |
| | /ApEinfo_fwdcolor=#84b0dc |
| misc_feature | complement(2142..2282) |
| | /label=“AAV2 ITR” |
| | /ApEinfo_revcolor=#ffef86 |
| | /ApEinfo_fwdcolor=#ffef86 |
| misc_feature | 4028..4033 |
| | /label=“cloning scar” |
| | /ApEinfo_revcolor=#ff9ccd |
| | /ApEinfo_fwdcolor=#ff9ccd |
| terminator | 5506..5709 |
| | /label=“bGH_PA_terminator” |
| | /ApEinfo_revcolor=#f58a5e |
| | /ApEinfo_fwdcolor=#f58a5e |
| misc_feature | 4145..4169 |
| | /label=“oMF66” |
| | /ApEinfo_revcolor=#b7e6d7 |
| | /ApEinfo_fwdcolor=#b7e6d7 |
| exon | 4731..4867 |
| | /label=“Atg5 exon” |
| | /ApEinfo_revcolor=#ff9ccd |
| | /ApEinfo_fwdcolor=#ff9ccd |
| exon | 4148..4275 |
| | /label=“Atg5 exon” |
| | /ApEinfo_revcolor=#b1ff67 |
| | /ApEinfo_fwdcolor=#b1ff67 |

ORIGIN

```
(SEQ ID NO: 16)
      1 ctacagagtt cttgaagtgg tggcctaact acggctacac tagaagaaca gtatttggta
    61 tctgcgctct gctgaagcca gttaccttcg gaaaaagagt tggtagctct tgatccggca
  121 aacaaaccac cgctggtagc ggtggttttt ttgtttgcaa gcagcagatt acgcgcagaa
  181 aaaaaggatc tcaagaagat cctttgatct tttctacggg gtctgacgct cagtggaacg
  241 aaaactcacg ttaagggatt ttggtcatga gattatcaaa aaggatcttc acctagatcc
  301 ttttaaatta aaaatgaagt tttaaatcaa tctaaagtat atatgagtaa acttggtctg
  361 acagttacca atgcttaatc agtgaggcac ctatctcagc gatctgtcta tttcgttcat
  421 ccatagttgc ctgactcccc gtcgtgtaga taactacgat acgggagggc ttaccatctg
  481 gccccagtgc tgcaatgata ccgcgagacc cacgctcacc ggctccagat ttatcagcaa
  541 taaaccagcc agccggaagg gccgagcgca gaagtggtcc tgcaacttta tccgcctcca
  601 tccagtctat taattgttgc cgggaagcta gagtaagtag ttcgccagtt aatagtttgc
  661 gcaacgttgt tgccattgct acaggcatcg tggtgtcacg ctcgtcgttt ggtatggctt
  721 cattcagctc cggttcccaa cgatcaaggc gagttacatg atcccccatg ttgtgcaaaa
  781 aagcggttag ctccttcggt cctccgatcg ttgtcagaag taagttggcc gcagtgttat
  841 cactcatggt tatggcagca ctgcataatt ctcttactgt catgccatcc gtaagatgct
  901 tttctgtgac tggtgagtac tcaaccaagt cattctgaga atagtgtatg cggcgaccga
  961 gttgctcttg cccggcgtca atacgggata ataccgcgcc acatagcaga actttaaaag
1021 tgctcatcat tggaaaacgt tcttcggggc gaaaactctc aaggatctta ccgctgttga
1081 gatccagttc gatgtaaccc actcgtgcac ccaactgatc ttcagcatct tttactttca
1141 ccagcgtttc tgggtgagca aaaacaggaa ggcaaaatgc cgcaaaaaag ggaataaggg
1201 cgacacggaa atgttgaata ctcatactct tcctttttca atattattga agcatttatc
1261 agggttattg tctcatgagc ggatacatat ttgaatgtat ttagaaaaat aaacaaatag
1321 gggttccgcg cacatttccc cgaaaagtgc cacctgacgt ctaagaaacc attattatca
1381 tgacattaac ctataaaaat aggcgtatca cgaggccctt tcgtctcgcg cgtttcggtg
1441 atgacggtga aaacctctga cacatgcagc tcccggagac ggtcacagct tgtctgtaag
1501 cggatgccgg gagcagacaa gcccgtcagg gcgcgtcagc gggtgttggc gggtgtcggg
1561 gctggcttaa ctatgcggca tcagagcaga ttgtactgag agtgcaccat aaaattgtaa
1621 acgttaatat tttgttaaaa ttcgcgttaa atttttgtta aatcagctca ttttttaacc
1681 aatagaccga aatcggcaaa atcccttata aatcaaaaga atagcccgag atagagttga
1741 gtgttgttcc agtttggaac aagagtccac tattaaagaa cgtggactcc aacgtcaaag
1801 ggcgaaaaac cgtctatcag ggcgatggcc cactacgtga accatcaccc aaatcaagtt
1861 ttttggggtc gaggtgccgt aaagcactaa atcggaaccc taaagggagc ccccgattta
1921 gagcttgacg gggaaagccg gcgaacgtgg cgagaaagga agggaagaaa gcgaaaggag
1981 cgggcgctaa ggcgctggca agtgtagcgg tcacgctgcg cgtaaccacc acacccgccg
2041 cgcttaatgc gccgctacag ggcgcgtact atggttgctt tgacgtatgc ggtgtgaaat
2101 accgcacaga tgcgtaagga gaaaataccg catcaggcgc ccctgcaggc agctgcgcgc
2161 tcgctcgctc actgaggccg cccgggcaaa gcccgggcgt cgggcgacct ttggtcgccc
2221 ggcctcagtg agcgagcgag cgcgcagaga gggagtggcc aactccatca ctaggggttc
2281 ctgcggccgc acgcgaaaca attctgcagg aatctagtta ttaatagtaa tcaattacgg
2341 ggtcattagt tcatagccca tatatggagt tccgcgttac ataacttacg gtaaatggcc
2401 cgcctggctg accgcccaac gacccccgcc cattgacgtc aataatgacg tatgttccca
2461 tagtaacgcc aatagggact ttccattgac gtcaatgggt ggagtattta cggtaaactg
2521 cccacttggc agtacatcaa gtgtatcata tgccaagtac gccccctatt gacgtcaatg
2581 acggtaaatg gcccgcctgg cattatgccc agtacatgac cttatgggac tttcctactt
2641 ggcagtacat ctacgtatta gtcatcgcta ttaccatggt cgaggtgagc cccacgttct
2701 gcttcactct ccccatctcc cccccctccc cacccccaat tttgtattta tttatttttt
2761 aattattttg tgcagcgatg ggggcggggg gggggggggg gcgcgcgcca ggcggggcgg
2821 ggcggggcga ggggcgggg cggggcgaggc ggagaggtgc ggcggcagcc aatcagagcg
2881 gcgcgctccg aaagtttcct tttatggcga ggcggcggcg gcggcggccc tataaaaagc
2941 gaagcgcgcg gcgggcggga gtcgctgcgc gctgccttcg ccccgtgccc cgctccgccg
3001 ccgcctcgcg ccgcccgccc cggctctgac tgaccgcgtt actcccacag gtgagcgggc
3061 gggacggccc ttctcctccg ggctgtaatt agcgcttggt ttaatgacgg cttgtttctt
3121 ttctgtggct gcgtgaaagc cttgaggggc tccgggaggg ccctttgtgc ggggggagcg
3181 gctcgggggg tgcgtgcgtg tgtgtgtgcg tggggagcgc cgcgtgcggc tccgcgctgc
3241 ccggcggctg tgagcgctgc gggcgcggcg cggggctttg tgcgctccgc agtgtgcgcg
3301 aggggagcgc ggccgggggc ggtgccccgc ggtgcggggg gggctgcgag gggaacaaag
3361 gctgcgtgcg gggtgtgtgc gtgggggggt gagcaggggg tgtgggcgcg tcggtcgggc
3421 tgcaaccccc cctgcacccc cctccccgag ttgctgagca cggcccggct tcgggtgcgg
3481 ggctccgtac ggggcgtggc gcggggctcg ccgtgccggg cggggggtgg cggcaggtgg
3541 gggtgccggg cggggcgggg ccgcctcggg ccggggaggg ctcgggggag gggcgcggcg
3601 gcccccggag cgccggcggc tgtcgaggcg cggcgagccg cagccattgc cttttatggt
3661 aatcgtgcga gagggcgcag ggacttcctt tgtcccaaat ctgtgcggag ccgaaatctg
3721 ggaggcgccg ccgcaccccc tctagcgggc gcggggcgaa gcggtgcggc gccggcagga
3781 aggaaatggg cggggagggc cttcgtgcgt cgccgcgccg ccgtcccctt ctccctctcc
3841 agcctcgggg ctgtccgcgg ggggacggct gccttcgggg gggacggggc agggcggggt
3901 tcggcttctg gcgtgtgacc ggcggctcta gagcctctgc taaccatgtt catgccttct
3961 tctttttcct acagctcctg ggcaacgtgc tggttattgt gctgtctcat cattttggca
4021 aagaattacg cgtgccacca tgacagatga caaagatgtg cttcgagatg tgtggtttgg
4081 acgaattcca acttgcttta ctctctatca ggatgagata actgaaagag aagcagaacc
4141 atactatttg cttttgccaa gagtcagcta tttgacgttg gtaactgaca aagtgaaaaa
4201 gcactttcag aaggttatga gacaagaaga tgttagtgag atatggtttg aatatgaagg
4261 cacacccctg aaatggcatt atccaattgg tttactattt gatcttcttg catcaagttc
4321 agctcttcct tggaacatca cagtacattt caagagtttt ccagaaaagg accttctaca
4381 ctgtccatcc aaggatgcgg ttgaggctca ctttatgtcg tgtatgaaag aagctgatgc
4441 tttaaagcat aaaagtcaag tgatcaacga aatgcagaaa aaagaccaca agcagctctg
4501 gatgggactg cagaatgaca gatttgacca gttttgggcc atcaaccgga aactcatgga
4561 atatcctcca gaagaaaatg gatttcgtta tatccccttt agaatatatc agaccacgac
4621 ggagcggcct ttcatccaga agctgttccg gcctgtggcc gcagatggac agctgcacac
4681 acttggagat ctcctcagag aagtctgtcc ttccgcagtc gcccctgaag atggagagaa
4741 gaggagccag gtgatgattc acgggataga gccaatgctg gaaacccctc tgcagtggct
4801 gagcgagcat ctgagctacc cagataactt tcttcatatt agcattgtcc cccagccaac
4861 agattgaaag cttatcgata atcaacctct ggattacaaa atttgtgaaa gattgactgg
4921 tattcttaac tatgttgctc cttttacgct atgtggatac gctgctttaa tgcctttgta
4981 tcatgctatt gcttcccgta tggctttcat tttctcctcc ttgtataaat cctggttgct
5041 gtctctttat gaggagttgt ggcccgttgt caggcaacgt ggcgtggtgt gcactgtgtt
5101 tgctgacgca acccccactg gttggggcat tgccaccacc tgtcagctcc tttccgggac
5161 tttcgctttc cccctcccta ttgccacggc ggaactcatc gccgcctgcc ttgcccgctg
5221 ctggacaggg gctcggctgt tgggcactga caattccgtg gtgttgtcgg ggaaatcatc
5281 gtcctttcct tggctgctcg cctatgttgc cacctggatt ctgcgcggga cgtccttctg
5341 ctacgtccct tcggccctca atccagcgga ccttccttcc cgcggcctgc tgccggctct
5401 gcggcctctt ccgcgtcttc gccttcgccc tcagacgagt cggatctccc tttgggccgc
5461 ctccccgcat cgataccgag cgctgctcga gagatcgatc tgcctcgact gtgccttcta
5521 gttgccagcc atctgttgtt tgcccctccc ccgtgccttc cttgaccctg gaaggtgcca
5581 ctcccactgt cctttcctaa taaaatgagg aaattgcatc gcattgtctg agtaggtgtc
5641 attctattct ggggggtggg gtggggcagg acagcaaggg ggaggattgg gaagacaata
5701 gcaggcatgc tggggacacg tgcggaccga gcggccgcag gaacccctag tgatggagtt
5761 ggccactccc tctctgcgcg ctcgctcgct cactgaggcc gggcgaccaa aggtcgcccg
5821 acgcccgggc tttgcccggg cggcctcagt gagcgagcga gcgcgcagca catgtgagca
5881 aaaggccagc aaaaggccag gaaccgtaaa aaggccgcgt tgctggcgtt tttccatagg
5941 ctccgccccc ctgacgagca tcacaaaaat cgacgctcaa gtcagaggtg gcgaaacccg
6001 acaggactat aaagatacca ggcgtttccc cctggaagct ccctcgtgcg ctctcctgtt
6061 ccgaccctgc cgcttaccgg atacctgtcc gcctttctcc cttcgggaag cgtggcgctt
6121 tctcatagct cacgctgtag gtatctcagt tcggtgtagg tcgttcgctc caagctgggc
6181 tgtgtgcacg aaccccccgt tcagcccgac cgctgcgcct tatccggtaa ctatcgtctt
6241 gagtccaacc cggtaagaca cgacttatcg ccactggcag cagccactgg taacaggatt
6301 agcagagcga ggtatgtagg cggtg
```

LOCUS AAV_pCAG-Cisd2-WPRE-bGH 5915 bp ds-DNA circular DEFINITION
KEYWORDS “accession:addgene_65455_110978”

| | |
|---|---|
| FEATURES | Location/Qualifiers |
| misc_feature | 5041..5069 |
| | /label=“AmpR promtoer” |
| | /ApEinfo_revcolor=#85dae9 |
| | /ApEinfo_fwdcolor=#85dae9 |
| misc_feature | 1631..1648 |
| | /label=“o2MF13 primer” |
| | /ApEinfo_revcolor=#75c6a9 |
| | /ApEinfo_fwdcolor=#75c6a9 |
| misc_feature | 617..639 |
| | /label=“oMF140” |
| | /ApEinfo_revcolor=#9eafd2 |
| | /ApEinfo_fwdcolor=#9eafd2 |
| misc_feature | 2213..2235 |
| | /label=“oMF155 Reverse” |
| | /ApEinfo_revcolor=#d59687 |
| | /ApEinfo_fwdcolor=#d59687 |
| misc_feature | 2532..2551 |
| | /label=“o2MF14 WPRE rev primer” |
| | /ApEinfo_revcolor=#b7e6d7 |
| | /ApEinfo_fwdcolor=#b7e6d7 |
| misc_feature | 817..1886 |
| | /label=“Chimeric intron” |
| | /ApEinfo_revcolor=#9eafd2 |
| | /ApEinfo_fwdcolor=#9eafd2 |
| misc_feature | 550..568 |
| | /label=“oMF139” |
| | /ApEinfo_revcolor=#f58a5e |
| | /ApEinfo_fwdcolor=#f58a5e |
| misc_feature | 243..268 |
| | /label=“21bp repeat element” |
| | /ApEinfo_revcolor=#75c6a9 |
| | /ApEinfo_fwdcolor=#75c6a9 |
| misc_feature | 3319..5915 |
| | /label=“AAV-CRE inverted backbone” |
| | /ApEinfo_revcolor=#75c6a9 |
| | /ApEinfo_fwdcolor=#75c6a9 |
| misc_feature | 2210..2233 |
| | /label=“oMF152 Forward” |
| | /ApEinfo_revcolor=#d6b295 |
| | /ApEinfo_fwdcolor=#d6b295 |
| misc_feature | 525..546 |
| | /label=“CAG3 R” |
| | /ApEinfo_revcolor=#ffef86 |
| | /ApEinfo_fwdcolor=#ffef86 |
| misc_feature | 474..503 |
| | /label=“oMF174 Probe CAG3 ” |
| | /ApEinfo_revcolor=#ff9ccd |
| | /ApEinfo_fwdcolor=#ff9ccd |
| misc_feature | 4139..4999 |
| | /label=“Ampicillin” |
| | /ApEinfo_revcolor=#d59687 |
| | /ApEinfo_fwdcolor=#d59687 |
| primer | complement(2942..2959) |
| | /label=“BGH_rev_primer” |
| | /ApEinfo_revcolor=#c7b0e3 |
| | /ApEinfo_fwdcolor=#c7b0e3 |
| misc_feature | 3178..3318 |
| | /label=“AAV2 ITR” |
| | /ApEinfo_revcolor=#ffef86 |
| | /ApEinfo_fwdcolor=#ffef86 |
| misc_feature | 757..774 |
| | /label=“oMF142” |
| | /ApEinfo_revcolor=#b4abac |
| | /ApEinfo_fwdcolor=#b4abac |
| misc_feature | complement(1..141) |
| | /label=“AAV2 ITR” |
| | /ApEinfo_revcolor=#ffef86 |
| | /ApEinfo_fwdcolor=#ffef86 |
| terminator | 2945..3148 |
| | /label=“bGH_PA_terminator” |
| | /ApEinfo_revcolor=#f58a5e |
| | /ApEinfo_fwdcolor=#f58a5e |
| misc_feature | 1860..1886 |
| | /label=“o2MF2 R binding site” |
| | /ApEinfo_revcolor=#b1ff67 |
| | /ApEinfo_fwdcolor=#b1ff67 |
| exon | 1899..2001 |
| | /label=“Cisd2 exon” |
| | /ApEinfo_revcolor=#b7e6d7 |
| | /ApEinfo_fwdcolor=#b7e6d7 |
| misc_feature | 2171..2195 |
| | /label=“oMF68” |
| | /ApEinfo_revcolor=#d6b295 |
| | /ApEinfo_fwdcolor=#d6b295 |
| exon | 2002..2216 |
| | /label=“Cisd2 exon” |
| | /ApEinfo_revcolor=#b7e6d7 |
| | /ApEinfo_fwdcolor=#b7e6d7 |
| misc_feature | 434..454 |
| | /label=“CAG3 F” |
| | /ApEinfo_revcolor=#d59687 |
| | /ApEinfo_fwdcolor=#d59687 |
| feature | 2321..2908 |
| | /label=“WPRE” |
| | /ApEinfo_revcolor=#b1ff67 |
| | /ApEinfo_fwdcolor=#b1ff67 |
| misc_feature | 2123..2147 |
| | /label=“oMF154-Forward” |
| | /ApEinfo_revcolor=#b4abac |
| | /ApEinfo_fwdcolor=#b4abac |
| misc_feature | 411..431 |
| | /label=“CAG4 reverse” |
| | /ApEinfo_revcolor=#d59687 |
| | /ApEinfo_fwdcolor=#d59687 |
| misc_feature | 623..642 |
| | /label=“oMF141\” |
| | /ApEinfo_revcolor=#ffef86 |
| | /ApEinfo_fwdcolor=#ffef86 |
| misc_feature | 2353..2387 |
| | /label=“WPRE R2” |
| | /ApEinfo_revcolor=#d59687 |
| | /ApEinfo_fwdcolor=#d59687 |
| misc_feature | 5199..5250 |
| | /label=“Sequence missing in original |
| | pCAG backbone” |
| | /ApEinfo_revcolor=#b1ff67 |
| | /ApEinfo_fwdcolor=#b1ff67 |
| misc_feature | 3365..3984 |
| | /label=“pBR322 origin” |
| | /ApEinfo_revcolor=#ff9ccd |
| | /ApEinfo_fwdcolor=#ff9ccd |
| misc_feature | 507..535 |
| | /label=“CMVfor” |
| | /ApEinfo_revcolor=#9eafd2 |
| | /ApEinfo_fwdcolor=#9eafd2 |
| misc_feature | 2076..2100 |
| | /label=“oMF67” |
| | /ApEinfo_revcolor=#f58a5e |
| | /ApEinfo_fwdcolor=#f58a5e |
| misc_feature | 539..815 |
| | /label=“CBA promoter” |
| | /ApEinfo_revcolor=#c7b0e3 |
| | /ApEinfo_fwdcolor=#c7b0e3 |
| misc_feature | 173..537 |
| | /label=“CMV enhancer” |
| | /ApEinfo_revcolor=#c7b0e3 |
| | /ApEinfo_fwdcolor=#c7b0e3 |
| misc_feature | 481..502 |
| | /label=“o2MF1 primer binding site” |
| | /ApEinfo_revcolor=#75c6a9 |
| | /ApEinfo_fwdcolor=#75c6a9 |
| misc_feature | 349..371 |
| | /label=“CAG4 forward” |
| | /ApEinfo_revcolor=#b4abac |
| | /ApEinfo_fwdcolor=#b4abac |
| misc_feature | 5518..5824 |
| | /label=“F1 origin” |
| | /ApEinfo_revcolor=#85dae9 |
| | /ApEinfo_fwdcolor=#85dae9 |
| primer | 1841..1860 |
| | /label=“pCAG_F_primer” |
| | /ApEinfo_revcolor=#b4abac |
| | /ApEinfo_fwdcolor=#b4abac |
| CDS | 1899..2306 |
| | /label=“Cisd2 CDS (CDGSH iron-sulfur |
| | domain-containing protein 2)” |
| | /ApEinfo_revcolor=#faac61 |
| | /ApEinfo_fwdcolor=#faac61 |
| misc_feature | 2010..2078 |
| | /label=“Cisd2 misc_feature” |
| | /ApEinfo_revcolor=#b1ff67 |
| | /ApEinfo_fwdcolor=#b1ff67 |
| misc_feature | 380..408 |
| | /label=“oMF175 CAG4 probe” |
| | /ApEinfo_revcolor=#9eafd2 |
| | /ApEinfo_fwdcolor=#9eafd2 |
| misc_feature | 1893..1898 |
| | /label=“Kozak” |
| | /ApEinfo_revcolor=#ff9ccd |
| | /ApEinfo_fwdcolor=#ff9ccd |
| misc_feature | 1860..1886 |
| | /label=“o2MF2 R binding site” |
| | /ApEinfo_revcolor=#f8d3a9 |
| | /ApEinfo_fwdcolor=#f8d3a9 |

ORIGIN

```
(SEQ ID NO: 17)
      1 cctgcaggca gctgcgcgct cgctcgctca ctgaggccgc ccgggcaaag cccgggcgtc
    61 gggcgacctt tggtcgcccg gcctcagtga gcgagcgagc gcgcagagag ggagtggcca
  121 actccatcac taggggttcc tgcggccgca cgcgaaacaa ttctgcagga atctagttat
  181 taatagtaat caattacggg gtcattagtt catagcccat atatggagtt ccgcgttaca
  241 taacttacgg taaatggccc gcctggctga ccgcccaacg acccccgccc attgacgtca
  301 ataatgacgt atgttcccat agtaacgcca atagggactt tccattgacg tcaatgggtg
  361 gagtatttac ggtaaactgc ccacttggca gtacatcaag tgtatcatat gccaagtacg
  421 ccccctattg acgtcaatga cggtaaatgg cccgcctggc attatgccca gtacatgacc
  481 ttatgggact ttcctacttg gcagtacatc tacgtattag tcatcgctat taccatggtc
  541 gaggtgagcc ccacgttctg cttcactctc cccatctccc ccccctcccc acccccaatt
  601 ttgtatttat ttatttttta attattttgt gcagcgatgg gggcgggggg gggggggggg
  661 cgcgcgccag gcggggcggg gcggggcgag gggcggggcg gggcgaggcg gagaggtgcg
  721 gcggcagcca atcagagcgg cgcgctccga aagtttcctt ttatggcgag gcggcggcgg
  781 cggcggccct ataaaaagcg aagcgcgcgg cgggcgggag tcgctgcgcg ctgccttcgc
  841 cccgtgcccc gctccgccgc cgcctcgcgc cgcccgcccc ggctctgact gaccgcgtta
  901 ctcccacagg tgagcgggcg ggacggccct tctcctccgg gctgtaatta gcgcttggtt
  961 taatgacggc ttgtttcttt tctgtggctg cgtgaaagcc ttgaggggct ccgggagggc
1021 cctttgtgcg gggggagcgg ctcggggggt gcgtgcgtgt gtgtgtgcgt ggggagcgcc
1081 gcgtgcggct ccgcgctgcc cggcggctgt gagcgctgcg ggcgcggcgc ggggctttgt
1141 gcgctccgca gtgtgcgcga ggggagcgcg gccgggggcg gtgccccgcg gtgcgggggg
1201 ggctgcgagg ggaacaaagg ctgcgtgcgg ggtgtgtgcg tgggggggtg agcagggggt
1261 gtgggcgcgt cggtcgggct gcaacccccc ctgcaccccc ctccccgagt tgctgagcac
1321 ggcccggctt cgggtgcggg gctccgtacg gggcgtggcg cggggctcgc cgtgccgggc
1381 ggggggtggc ggcaggtggg ggtgccgggc ggggcggggc cgcctcgggc cggggagggc
1441 tcgggggagg ggcgcggcgg cccccggagc gccggcggct gtcgaggcgc ggcgagccgc
1501 agccattgcc ttttatggta atcgtgcgag agggcgcagg gacttccttt gtcccaaatc
1561 tgtgcggagc cgaaatctgg gaggcgccgc cgcaccccct ctagcgggcg cggggcgaag
1621 cggtgcggcg ccggcaggaa ggaaatgggc ggggagggcc ttcgtgcgtc gccgcgccgc
1681 cgtccccttc tccctctcca gcctcggggc tgtccgcggg gggacggctg ccttcggggg
1741 ggacggggca gggcggggtt cggcttctgg cgtgtgaccg gcggctctag agcctctgct
1801 aaccatgttc atgccttctt ctttttccta cagctcctgg gcaacgtgct ggttattgtg
1861 ctgtctcatc attttggcaa agaattacgc gtgccaccat ggtcctggac agcgtggccc
1921 gcatcgtgaa ggtgcagctg cccgcctacc tcaagcagct cccggtcccc gacagcatca
1981 ccgggttcgc ccgcctcaca gtttcagact ggctccgcct actgcccttc ctcggggtac
2041 ttgcgcttct gggctacctc gcagtgcgcc cattcttccc aaagaagaag caacagaagg
2101 atagcttgat caatcttaag atacaaaagg aaaatcccaa ggtggtgaat gagataaaca
2161 ttgaagatct gtgtctcacc aaagcagctt attgtaggtg ctggcggtcc aagacgtttc
2221 ctgcctgtga tggatcccat aataagcata atgaattgac aggcgataac gtgggtcctc
2281 tcatcctgaa gaagaaagaa gtatagaagc ttatcgataa tcaacctctg gattacaaaa
2341 tttgtgaaag attgactggt attcttaact atgttgctcc ttttacgcta tgtggatacg
2401 ctgctttaat gcctttgtat catgctattg cttcccgtat ggctttcatt ttctcctcct
2461 tgtataaatc ctggttgctg tctctttatg aggagttgtg gcccgttgtc aggcaacgtg
2521 gcgtggtgtg cactgtgttt gctgacgcaa cccccactgg ttggggcatt gccaccacct
2581 gtcagctcct ttccgggact ttcgctttcc ccctccctat tgccacggcg gaactcatcg
2641 ccgcctgcct tgcccgctgc tggacagggg ctcggctgtt gggcactgac aattccgtgg
2701 tgttgtcggg gaaatcatcg tcctttcctt ggctgctcgc ctatgttgcc acctggattc
2761 tgcgcgggac gtccttctgc tacgtccctt cggccctcaa tccagcggac cttccttccc
2821 gcggcctgct gccggctctg cggcctcttc cgcgtcttcg ccttcgccct cagacgagtc
2881 ggatctccct ttgggccgcc tccccgcatc gataccgagc gctgctcgag agatcgatct
2941 gcctcgactg tgccttctag ttgccagcca tctgttgttt gcccctcccc cgtgccttcc
3001 ttgaccctgg aaggtgccac tcccactgtc ctttcctaat aaaatgagga aattgcatcg
3061 cattgtctga gtaggtgtca ttctattctg gggggtgggg tggggcagga cagcaagggg
3121 gaggattggg aagacaatag caggcatgct ggggacacgt gcggaccgag cggccgcagg
3181 aacccctagt gatggagttg gccactccct ctctgcgcgc tcgctcgctc actgaggccg
3241 ggcgaccaaa ggtcgcccga cgcccgggct ttgcccgggc ggcctcagtg agcgagcgag
3301 cgcgcagctg cctgcaggac atgtgagcaa aaggccagca aaaggccagg aaccgtaaaa
3361 aggccgcgtt gctggcgttt ttccataggc tccgcccccc tgacgagcat cacaaaaatc
3421 gacgctcaag tcagaggtgg cgaaacccga caggactata aagataccag gcgtttcccc
3481 ctggaagctc cctcgtgcgc tctcctgttc cgaccctgcc gcttaccgga tacctgtccg
3541 cctttctccc ttcgggaagc gtggcgcttt ctcatagctc acgctgtagg tatctcagtt
3601 cggtgtaggt cgttcgctcc aagctgggct gtgtgcacga accccccgtt cagcccgacc
3661 gctgcgcctt atccggtaac tatcgtcttg agtccaaccc ggtaagacac gacttatcgc
3721 cactggcagc agccactggt aacaggatta gcagagcgag gtatgtaggc ggtgctacag
3781 agttcttgaa gtggtggcct aactacggct acactagaag aacagtattt ggtatctgcg
3841 ctctgctgaa gccagttacc ttcggaaaaa gagttggtag ctcttgatcc ggcaaacaaa
3901 ccaccgctgg tagcggtggt ttttttgttt gcaagcagca gattacgcgc agaaaaaaag
3961 gatctcaaga agatcctttg atcttttcta cggggtctga cgctcagtgg aacgaaaact
4021 cacgttaagg gattttggtc atgagattat caaaaaggat cttcacctag atccttttaa
4081 attaaaaatg aagttttaaa tcaatctaaa gtatatatga gtaaacttgg tctgacagtt
4141 accaatgctt aatcagtgag gcacctatct cagcgatctg tctatttcgt tcatccatag
4201 ttgcctgact ccccgtcgtg tagataacta cgatacggga gggcttacca tctggcccca
4261 gtgctgcaat gataccgcga gacccacgct caccggctcc agatttatca gcaataaacc
4321 agccagccgg aagggccgag cgcagaagtg gtcctgcaac tttatccgcc tccatccagt
4381 ctattaattg ttgccgggaa gctagagtaa gtagttcgcc agttaatagt ttgcgcaacg
4441 ttgttgccat tgctacaggc atcgtggtgt cacgctcgtc gtttggtatg gcttcattca
4501 gctccggttc ccaacgatca aggcgagtta catgatcccc catgttgtgc aaaaaagcgg
4561 ttagctcctt cggtcctccg atcgttgtca gaagtaagtt ggccgcagtg ttatcactca
4621 tggttatggc agcactgcat aattctctta ctgtcatgcc atccgtaaga tgcttttctg
4681 tgactggtga gtactcaacc aagtcattct gagaatagtg tatgcggcga ccgagttgct
4741 cttgcccggc gtcaatacgg gataataccg cgccacatag cagaacttta aaagtgctca
4801 tcattggaaa acgttcttcg gggcgaaaac tctcaaggat cttaccgctg ttgagatcca
4861 gttcgatgta acccactcgt gcacccaact gatcttcagc atcttttact ttcaccagcg
4921 tttctgggtg agcaaaaaca ggaaggcaaa atgccgcaaa aaagggaata agggcgacac
4981 ggaaatgttg aatactcata ctcttccttt ttcaatatta ttgaagcatt tatcagggtt
5041 attgtctcat gagcggatac atatttgaat gtatttagaa aaataaacaa ataggggttc
5101 cgcgcacatt tccccgaaaa gtgccacctg acgtctaaga aaccattatt atcatgacat
5161 taacctataa aaataggcgt atcacgaggc cctttcgtct cgcgcgtttc ggtgatgacg
5221 gtgaaaacct ctgacacatg cagctcccgg agacggtcac agcttgtctg taagcggatg
5281 ccgggagcag acaagcccgt cagggcgcgt cagcgggtgt tggcgggtgt cggggctggc
5341 ttaactatgc ggcatcagag cagattgtac tgagagtgca ccataaaatt gtaaacgtta
5401 atattttgtt aaaattcgcg ttaaattttt gttaaatcag ctcatttttt aaccaataga
5461 ccgaaatcgg caaaatccct tataaatcaa aagaatagcc cgagatagag ttgagtgttg
5521 ttccagtttg gaacaagagt ccactattaa agaacgtgga ctccaacgtc aaagggcgaa
5581 aaaccgtcta tcagggcgat ggcccactac gtgaaccatc acccaaatca agttttttgg
5641 ggtcgaggtg ccgtaaagca ctaaatcgga accctaaagg gagcccccga tttagagctt
5701 gacggggaaa gccggcgaac gtggcgagaa aggaagggaa gaaagcgaaa ggagcgggcg
5761 ctaaggcgct ggcaagtgta gcggtcacgc tgcgcgtaac caccacaccc gccgcgctta
5821 atgcgccgct acagggcgcg tactatggtt gctttgacgt atgcggtgtg aaataccgca
5881 cagatgcgta aggagaaaat accgcatcag gcgcc
```

### REFERENCES

1. Niccoli, T. et al. Ageing as a risk factor for disease. Curr. Biol. 22, R741-52 (2012). PMID: 22975005
2. CDC. The State of Aging and Health in America 2013. (2013).
3. de Magalhães, J. P. et al. The Human Ageing Genomic Resources: online databases and tools for biogerontologists. Aging Cell 8, 65-72 (2009). PMID: 18986374
4. Moskalev, A. et al. Geroprotectors.org: a new, structured and curated database of current therapeutic interventions in aging and age-related disease. Aging (Albany, N.Y.). 7, 616-628 (2015). PMID: 26342919
5. Rigoli, L. & Di Bella, C. Wolfram syndrome 1 and Wolfram syndrome 2. Curr. Opin. Pediatr. 24, 1 (2012). PMID: 22790102
6. Urano, F. Wolfram Syndrome: Diagnosis, Management, and Treatment. Curr. Diab. Rep. 16, 6 (2016). PMID: 26742931
7. Chen, Y.-F., Wu, C.-Y., Kirby, R., Kao, C.-H. & Tsai, T.-F. A role for the CISD2 gene in lifespan control and human disease. Ann. N.Y. Acad. Sci. 1201, 58-64 (2010). PMID: 20649540
8. Conlan, A. R. et al. Crystal structure of Minerl: The redox-active 2Fe-2S protein causative in Wolfram Syndrome 2. J. Mol. Biol. 392, 143-53 (2009). PMID: 19580816
9. Chen, Y.-F. et al. Cisd2 deficiency drives premature aging and causes mitochondria-mediated defects in mice. Genes Dev. 23, 1183-1194 (2009). PMID: 19451219
10. Wu, C.-Y. et al. A persistent level of Cisd2 extends healthy lifespan and delays aging in mice. Hum. Mol. Genet. 21, 3956-3968 (2012). PMID: 22661501
11. Puca, A. A. et al. A genome-wide scan for linkage to human exceptional longevity identifies a locus on chromosome 4. Proc. Natl. Acad. Sci. 98, 10505-10508 (2001). PMID: 11526246
12. Lu, Y. et al. Reversal of ageing- and injury-induced vision loss by Tet-dependent epigenetic reprogramming. bioRxiv 710210 (2019). doi:10.1101/710210
13. Zincarelli, C., Soltys, S., Rengo, G. & Rabinowitz, J. E. Analysis of AAV serotypes 1-9 mediated gene expression and tropism in mice after systemic injection. Mol. Ther. 16, 1073-1080 (2008). PMID: 18414476
14. Whitehead, J. C. et al. A clinical frailty index in aging mice: Comparisons with frailty index data in humans. Journals Gerontol.—Ser. A Biol. Sci. Med. Sci. 69, 621-632 (2014). PMID: 24051346
15. Willeit, P., Skroblin, P., Kiechl, S., Fernandez-Hernando, C. & Mayr, M. Liver microRNAs: potential mediators and biomarkers for metabolic and cardiovascular disease? Eur. Heart J. 37, 3260-3266 (2016). PMID: 27099265
16. Xu, C. et al. The muscle-specific microRNAs miR-1 and miR-133 produce opposing effects on apoptosis by targeting HSP60, HSP70 and caspase-9 in cardiomyocytes. J. Cell Sci. 120, 3045-3052 (2007). PMID: 17715156
17. Chistiakov, D. A., Orekhov, A. N. & Bobryshev, Y. V. Cardiac-specific miRNA in cardiogenesis, heart function, and cardiac pathology (with focus on myocardial infarction). J. Mol. Cell. Cardiol. 94, 107-121 (2016). doi:10.1016/j.yjmcc.2016.03.015
18. Cao, X., Pfaff, S. L. & Gage, F. H. A functional study of miR-124 in the developing neural tube. Genes Dev. 21, 531-536 (2007). doi:10.1101/gad.1519207
19. Adlakha, Y. K. & Saini, N. Brain microRNAs and insights into biological functions and therapeutic potential of brain enriched miRNA-128. Molecular Cancer 13, 33 (2014). doi:10.1186/1476-4598-13-33
20. De Rie, D. et al. An integrated expression atlas of miRNAs and their promoters in human and mouse. Nat. Biotechnol. 35, 872-878 (2017). PMID: 28829439

What is claimed is:

1. A viral vector delivery system comprising two or more adeno-associated viral serotypes engineered for delivery of a single gene; a miRNA target site selected based on a tissue target; and a non-silencing promoter.

2. The viral vector delivery system of claim 1, wherein the two or more viral serotypes are selected from the group consisting of AAV8, AAV9, Anc80, AAV-DJ, AAV-PHP.S, AAV-PHP.eB, AAV.CAP-B10, AAV.CAP-B22, and AAVMYO.

3. The viral vector delivery system of claim 1, wherein at least one viral serotype comprises AAV9 or PHP.eB.

4. The viral vector delivery system of claim 1, wherein the two or more viral serotypes include AAV9 and PHP.eB.

5. The viral vector delivery system of claim 1, wherein the tissue target is selected from the group consisting of aorta, endothelium, cardiac muscle skeletal muscle, tongue, esophagus, stomach, small intestine, large intestine, diaphragm, eye, optic nerve, inner ear, auditory nerve, brown fat, white fat, central nervous system, peripheral nervous system, kidney, spleen, liver, lung, heart, brain, thymus, ovaries, testes, skin, pancreas, bone marrow cells, osteoblasts and osteoclasts, blood cells, hematopoietic stem cells, and muscle satellite cells.

6. The viral vector delivery system of claim 1, wherein the miRNA target site is selected from the group consisting of miRNA-1, miRNA-24, miRNA-29, miRNA-30c, miRNA-33, miRNA-122, miRNA-124, miRNA-128, miRNA-133, miRNA-144, miRNA-148a, miRNA-208a, miRNA-208b, miRNA-223, and miRNA-499.

7. The viral vector delivery system of claim 1, wherein a target tissue is cardiac tissue and the miRNA target site is selected from the group consisting of miRNA-1, miRNA-133, miRNA-208a, miRNA-208b, and miRNA-499;
wherein a target tissue is liver tissue and the miRNA target site is selected from the group consisting of miRNA-24, miRNA-29, miRNA-30c, miRNA-33, miRNA-122, miRNA-144, miRNA-148a, and miRNA-223;
wherein a target tissue is muscle tissue and the miRNA target site is miRNA-1 or miRNA-133; or
wherein a target tissue is brain tissue and the miRNA target site is miRNA-124 or miRNA-128.

8. The viral vector delivery system of claim 1, wherein the non-silencing promoter leads to RNA expression of at least 30% of CMV promoter expression; or
wherein the non-silencing promoter leads to RNA expression of at least 50% of CMV promoter expression.

9. The viral vector delivery system of claim 1, wherein the promoter is selected from the group consisting of Cbh, CAG, CB7, and CBA.

10. The viral vector delivery system of claim 1 further comprising a self-complementary vector backbone.

11. The viral vector delivery system of claim 1, wherein the gene is selected from the group consisting of Cisd2, Atg5, and PTEN.

12. A pharmaceutical composition comprising the viral vector delivery system of claim 1.

13. A method of treating a disease or disorder in a subject comprising administering the pharmaceutical composition of claim 12 to the subject.

14. A method of extending the lifespan of a subject comprising administering the pharmaceutical composition of claim 12 to the subject.

15. A method of treating or preventing a disease or disorder comprising administering to a subject a viral vector delivery system comprising at least two adeno-associated viral serotypes engineered for delivery of a single gene; an miRNA target site selected based on a tissue target; and a non-silencing promoter.

16. The method of claim 15, wherein the two or more viral serotypes are selected from the group consisting of AAV8, AAV9, Anc80, AAV-DJ, AAV-PHP.S, AAV-PHP.eB, AAV.CAP-B10, AAV.CAP-B22, and AAVMYO.

17. The method of claim 15, wherein at least one viral serotype comprises AAV9 or PHP.eB.

18. The method of claim 15, wherein the two or more viral serotypes include AAV9 and PHP.eB.

19. The method of claim 15, wherein the tissue target is selected from the group consisting of aorta, endothelium, cardiac muscle skeletal muscle, tongue, esophagus, stomach, small intestine, large intestine, diaphragm, eye, optic nerve, inner ear, auditory nerve, brown fat, white fat, central nervous system, peripheral nervous system, kidney, spleen, liver, lung, heart, brain, thymus, ovaries, testes, skin, pancreas, bone marrow cells, osteoblasts and osteoclasts, blood cells, hematopoietic stem cells, and muscle satellite cells.

20. The method of claim 15, wherein the miRNA target site is selected from the group consisting of miRNA-1, miRNA-24, miRNA-29, miRNA-30c, miRNA-33, miRNA-122, miRNA-124, miRNA-128, miRNA-133, miRNA-144, miRNA-148a, miRNA-208a, miRNA-208b, miRNA-223, and miRNA-499.

21. The method of claim 15, wherein a target tissue is cardiac tissue and the miRNA target site is selected from the group consisting of miRNA-1, miRNA-133, miRNA-208a, miRNA-208b, and miRNA-499;
wherein a target tissue is liver tissue and the miRNA target site is selected from the group consisting of miRNA-24, miRNA-29, miRNA-30c, miRNA-33, miRNA-122, miRNA-144, miRNA-148a, and miRNA-223;
wherein a target tissue is muscle tissue and the miRNA target site is miRNA-1 or miRNA-133; or
wherein a target tissue is brain tissue and the miRNA target site is miRNA-124 or miRNA-128.

22. The method of claim 15, wherein the non-silencing promoter leads to RNA expression of at least 30% of CMV promoter expression; or
wherein the non-silencing promoter leads to RNA expression of at least 50% of CMV promoter expression.

23. The method of claim 15, wherein the promoter is selected from the group consisting of Cbh, CAG, CB7, and CBA.

24. The method of claim 15, wherein the viral vector delivery system further comprises a self-complementary vector backbone.

25. The method of claim 15, wherein the gene is selected from the group consisting of Cisd2, Atg5, and PTEN.

26. The method of claim 15, wherein the disease or disorder is an aging related disease or disorder.

27. The method of claim 15, wherein the disease or disorder is selected from the group consisting of progeria syndrome, Wolfram Syndrome, neurodegenerative disorder, neurovascular disorder, skeletal muscle conditions, Cowden syndrome, Bannayan-Riley-Ruvalcaba syndrome, Proteus syndrome, Proteus-like syndrome and other PTEN-opathies. Werner syndrome, Bloom syndrome, Rothmund-Thomson syndrome, Cockayne syndrome, xeroderma pigmentosum, trichothiodystrophy, combined xeroderma pigmentosum-Cockayne syndrome, Hutchinson-Gilford Progeria syndrome, restrictive dermopathy, diabetes, obesity, cardiovascular disease, cancer, ocular degeneration, liver failure, and age-related macular degeneration.

28. The method of claim 15, wherein the gene is expressed in two or more tissues in the subject.

29. A viral vector delivery system comprising two or more AAV serotypes engineered for delivery of a single gene, a non-silencing promoter, at least one miRNA target site, the gene, and optionally a self-complementary backbone.

30. The viral vector delivery system of claim 29, wherein the AAV serotypes are AAV9 and PHP.eB; and wherein the gene is selected from the group consisting of Cisd2, Atg5, and PTEN.


SEQUENCE LISTING (17 sequences)

SEQ ID NO: 1 - DNA, 16 bp, Artificial
  primer
gccagccatctgttgt

SEQ ID NO: 2 - DNA, 16 bp, Artificial
  primer
ggagtggcaccttcca

SEQ ID NO: 3 - DNA, 21 bp, Artificial
  primer
tcccccgtgccttccttgacc

SEQ ID NO: 4 - DNA, 23 bp, Artificial
  primer
gttttcatcactgttcatgatgc

SEQ ID NO: 5 - DNA, 22 bp, Artificial
  primer
tcatgggcattactattcctac

SEQ ID NO: 6 - DNA, 35 bp, Artificial
  primer
aggaccagcttctctgcattatcatcgttgaagat

SEQ ID NO: 7 - DNA, 20 bp, Artificial
  Guide RNA
agcgcaagtaccccgaggaa

SEQ ID NO: 8 - DNA, 20 bp, Artificial
  Guide RNA
ccccgaggaagggcagtagg

SEQ ID NO: 9 - DNA, 20 bp, Artificial
  Guide RNA
tgctgtgttcagtttcagac

SEQ ID NO: 10 - DNA, 22 bp, Artificial
  Primer
agccctaagtttctccgagttc

SEQ ID NO: 11 - DNA, 22 bp, Artificial
  Primer
gtgacatgtggtgctgtagaac

SEQ ID NO: 12 - DNA, 5237 bp, Artificial
  Viral vector
gttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagggatctgggccactccctctctgcgcgctcgctcgctcactgaggccgggcgaccaaaggtcgcccgacgcccgggctttgcccgggcggcctcagtgagcgagcgagcgcgcagagagggagtggttaagctagcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagacgcgtgccaccatgacggatgataaagatgttctgagagatgtctggttcggacgcattcctacctgcttcacgctgtaccaagatgagattacggagagggaggctgaaccctactacctgctgctgccaagagtcagctacctgactctggtgaccgacaaggtcaagaagcacttccagaaggtcatgaggcaggaggacgtgtctgaaatctggttcgagtacgaaggaactcctctgaagtggcactaccccatcggtctgctgttcgacctgctggcttccagctctgccctgccttggaacatcaccgtccacttcaagagcttcccagagaaggacctgctgcactgcccttcaaaggacgctgtggaggcccacttcatgtcctgcatgaaggaagctgacgccctgaagcacaagtcccaggtcatcaacgaaatgcagaagaaggaccacaagcagctgtggatgggtctgcaaaacgaccgcttcgaccagttctgggctatcaaccgtaagctgatggagtaccctcctgaggaaaacggcttccgctacatccccttccgtatctaccagaccactaccgaaaggcccttcatccagaagctgttcagaccagtggctgccgacggtcagctgcacactctgggcgacctgctgagggaggtctgcccatcagctgtggctcctgaggacggagaaaagaggagccaggtcatgatccacggaatcgagccaatgctggaaacccctctgcaatggctgtccgaacacctctcctacccagacaacttcctccacatttccattgtcccccaacctacggactaaaagcttatcgcgaacaaacaccattgtcacactccaactagtataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 13 - DNA, 4817 bp, Artificial
  Viral vector
ctgcgcgctcgctcgctcactgaggccgggcgaccaaaggtcgcccgacgcccgggctttgcccgggcggcctcagtgagcgagcgagcgcgcagagagggagtggttaagctagcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagacgcgtgccaccatggtcctggacagcgtggcccgcatcgtgaaggtgcagctgcccgcctacctcaagcagctcccggtccccgacagcatcaccgggttcgcccgcctcacagtttcagactggctccgcctactgcccttcctcggggtacttgcgcttctgggctacctcgcagtgcgcccattcttcccaaagaagaagcaacagaaggatagcttgatcaatcttaagatacaaaaggaaaatcccaaggtggtgaatgagataaacattgaagatctgtgtctcaccaaagcagcttattgtaggtgctggcggtccaagacgtttcctgcctgtgatggatcccataataagcataatgaattgacaggcgataacgtgggtcctctcatcctgaagaagaaagaagtatagaagcttatcgcgaacaaacaccattgtcacactccaactagtataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagggatctgggccactccctct

SEQ ID NO: 14 - DNA, 5129 bp, Artificial
  Viral vector
gttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagggatctgggccactccctctctgcgcgctcgctcgctcactgaggccgggcgaccaaaggtcgcccgacgcccgggctttgcccgggcggcctcagtgagcgagcgagcgcgcagagagggagtggttaagctagcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagacgcgtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttatcgcgaacaaacaccattgtcacactccaactagtataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 15 - DNA, 5283 bp, Artificial
  Viral vector
gttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagggatctgggccactccctctctgcgcgctcgctcgctcactgaggccgggcgaccaaaggtcgcccgacgcccgggctttgcccgggcggcctcagtgagcgagcgagcgcgcagagagggagtggttaagctagcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagacgcgtgccaccatgacagccatcatcaaagagatcgttagcagaaacaaaaggagatatcaagaggatggattcgacttagacttgacctatatttatccaaatattattgctatgggatttcctgcagaaagacttgaaggtgtatacaggaacaatattgatgatgtagtaaggtttttggattcaaagcataaaaaccattacaagatatacaatctatgtgctgagagacattatgacaccgccaaatttaactgcagagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaagggagtcacaattcccagtcagaggcgctatgtatattattatagctacctgctaaaaaatcacctggattacagacccgtggcactgctgtttcacaagatgatgtttgaaactattccaatgttcagtggcggaacttgcaatcctcagtttgtggtctgccagctaaaggtgaagatatattcctccaattcaggacccacgcggcgggaggacaagttcatgtactttgagttccctcagccattgcctgtgtgtggtgatatcaaagtagagttcttccacaaacagaacaagatgctcaaaaaggacaaaatgtttcacttttgggtaaatacgttcttcataccaggaccagaggaaacctcagaaaaagtggaaaatggaagtctttgtgatcaggaaatcgatagcatttgcagtatagagcgtgcagataatgacaaggagtatcttgtactcaccctaacaaaaaacgatcttgacaaagcaaacaaagacaaggccaaccgatacttctctccaaattttaaggtgaaactatactttacaaaaacagtagaggagccatcaaatccagaggctagcagttcaacttctgtgactccagatgttagtgacaatgaacctgatcattatagatattctgacaccactgactctgatccagagaatgaaccttttgatgaagatcagcattcacaaattacaaaagtctgataaaagcttatcgcgaacaaacaccattgtcacactccaactagttaagatacattgatgagtttggacaaaccacaactagaatgcagtgaaaaaaatgctttatttgtgaaatttgtgatgctattgctttatttgtaaccattataagctgcaataaacaagtaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgg

SEQ ID NO: 16 - DNA, 6325 bp, Artificial
  Viral vector
ctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgaaacaattctgcaggaatctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacgcgtgccaccatgacagatgacaaagatgtgcttcgagatgtgtggtttggacgaattccaacttgctttactctctatcaggatgagataactgaaagagaagcagaaccatactatttgcttttgccaagagtcagctatttgacgttggtaactgacaaagtgaaaaagcactttcagaaggttatgagacaagaagatgttagtgagatatggtttgaatatgaaggcacacccctgaaatggcattatccaattggtttactatttgatcttcttgcatcaagttcagctcttccttggaacatcacagtacatttcaagagttttccagaaaaggaccttctacactgtccatccaaggatgcggttgaggctcactttatgtcgtgtatgaaagaagctgatgctttaaagcataaaagtcaagtgatcaacgaaatgcagaaaaaagaccacaagcagctctggatgggactgcagaatgacagatttgaccagttttgggccatcaaccggaaactcatggaatatcctccagaagaaaatggatttcgttatatcccctttagaatatatcagaccacgacggagcggcctttcatccagaagctgttccggcctgtggccgcagatggacagctgcacacacttggagatctcctcagagaagtctgtccttccgcagtcgcccctgaagatggagagaagaggagccaggtgatgattcacgggatagagccaatgctggaaacccctctgcagtggctgagcgagcatctgagctacccagataactttcttcatattagcattgtcccccagccaacagattgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatcgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtg

SEQ ID NO: 17 - DNA, 5915 bp, Artificial
  Viral vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgaaacaattctgcaggaatctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacgcgtgccaccatggtcctggacagcgtggcccgcatcgtgaaggtgcagctgcccgcctacctcaagcagctcccggtccccgacagcatcaccgggttcgcccgcctcacagtttcagactggctccgcctactgcccttcctcggggtacttgcgcttctgggctacctcgcagtgcgcccattcttcccaaagaagaagcaacagaaggatagcttgatcaatcttaagatacaaaaggaaaatcccaaggtggtgaatgagataaacattgaagatctgtgtctcaccaaagcagcttattgtaggtgctggcggtccaagacgtttcctgcctgtgatggatcccataataagcataatgaattgacaggcgataacgtgggtcctctcatcctgaagaagaaagaagtatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatcgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc